SEQUENCE LISTING (83 sequences)

SEQ ID NO: 1 - DNA, 26 bp, Artificial sequence
  Synthetic oligonucleotide
tgacatcaagaaggtggtgaagcagg

SEQ ID NO: 2 - DNA, 23 bp, Artificial sequence
  Synthetic oligonucleotide
gcgtcaaaggtggaggagtgggt

SEQ ID NO: 3 - DNA, 20 bp, Artificial sequence
  Synthetic oligonucleotide
ctgggttgatcctcggacct

SEQ ID NO: 4 - DNA, 19 bp, Artificial sequence
  Synthetic oligonucleotide
ccatcggagttgctctcca

SEQ ID NO: 5 - DNA, 20 bp, Artificial sequence
  Synthetic oligonucleotide
gatttgtgggcctgaagaaa

SEQ ID NO: 6 - DNA, 20 bp, Artificial sequence
  Synthetic oligonucleotide
cagatccatggaggaaggaa

SEQ ID NO: 7 - DNA, 26 bp, Artificial sequence
  Synthetic oligonucleotide
ttgcgtgagtgtggatgggattggtg

SEQ ID NO: 8 - DNA, 26 bp, Artificial sequence
  Synthetic oligonucleotide
gggaaatgggaggggtgcaaaagagg

SEQ ID NO: 9 - DNA, 22 bp, Artificial sequence
  Synthetic oligonucleotide
cgaagaagagccggcctgtacc

SEQ ID NO: 10 - DNA, 22 bp, Artificial sequence
  Synthetic oligonucleotide
cggaagcccatgcaacgatctc

SEQ ID NO: 11 - DNA, 22 bp, Artificial sequence
  Synthetic oligonucleotide
gctaacatctgccaccccacca

SEQ ID NO: 12 - DNA, 22 bp, Artificial sequence
  Synthetic oligonucleotide
ggattctgcggtgctgctgaca

SEQ ID NO: 13 - DNA, 21 bp, Artificial sequence
  Synthetic oligonucleotide
cgccgctacaagttccttaaa

SEQ ID NO: 14 - DNA, 19 bp, Artificial sequence
  Synthetic oligonucleotide
ggatctgctcgtcgaaggg

SEQ ID NO: 15 - DNA, 21 bp, Artificial sequence
  Synthetic oligonucleotide
agaaacgggcaaagacaagac

SEQ ID NO: 16 - DNA, 21 bp, Artificial sequence
  Synthetic oligonucleotide
gctgacaggttctatttccgc

SEQ ID NO: 17 - DNA, 23 bp, Artificial sequence
  Synthetic oligonucleotide
ttgaggaagagaatcagatccag

SEQ ID NO: 18 - DNA, 20 bp, Artificial sequence
  Synthetic oligonucleotide
cgttctgtacggggtcactc

SEQ ID NO: 19 - DNA, 19 bp, Artificial sequence
  Synthetic oligonucleotide
caggagctgtcgtgcaagt

SEQ ID NO: 20 - DNA, 21 bp, Artificial sequence
  Synthetic oligonucleotide
agtagcagacgtggttgttct

SEQ ID NO: 21 - DNA, 21 bp, Artificial sequence
  Synthetic oligonucleotide
tttccctcattcctgaactgc

SEQ ID NO: 22 - DNA, 20 bp, Artificial sequence
  Synthetic oligonucleotide
cagcaaaatcaaccatcaaa

SEQ ID NO: 23 - DNA, 20 bp, Artificial sequence
  Synthetic oligonucleotide
agcacatcaactcggaggag

SEQ ID NO: 24 - DNA, 21 bp, Artificial sequence
  Synthetic oligonucleotide
cattccctgggtggttcactg

SEQ ID NO: 25 - DNA, 22 bp, Artificial sequence
  Synthetic oligonucleotide
cagaagtcctacaaggtgtcca

SEQ ID NO: 26 - DNA, 21 bp, Artificial sequence
  Synthetic oligonucleotide
ctctggttgaccgtaactgcg

SEQ ID NO: 27 - DNA, 22 bp, Artificial sequence
  Synthetic oligonucleotide
tcgcaaatactgtggacaatgc

SEQ ID NO: 28 - DNA, 21 bp, Artificial sequence
  Synthetic oligonucleotide
gcagtcgtgtgatattggtgt

SEQ ID NO: 29 - DNA, 21 bp, Artificial sequence
  Synthetic oligonucleotide
accaagtttgagacggaacag

SEQ ID NO: 30 - DNA, 21 bp, Artificial sequence
  Synthetic oligonucleotide
ccctcagcgtactgatttcct

SEQ ID NO: 31 - DNA, 24 bp, Artificial sequence
  Synthetic oligonucleotide
aggaaatcctcagactcctgggtt

SEQ ID NO: 32 - DNA, 24 bp, Artificial sequence
  Synthetic oligonucleotide
cccaaactgttcaagtggcagaca

SEQ ID NO: 33 - DNA, 20 bp, Artificial sequence
  Synthetic oligonucleotide
ccctgggatcgagtgaagga

SEQ ID NO: 34 - DNA, 21 bp, Artificial sequence
  Synthetic oligonucleotide
ctgggacacatagtctctgcc

SEQ ID NO: 35 - DNA, 21 bp, Artificial sequence
  Synthetic oligonucleotide
ctgtgctactcctcaccatct

SEQ ID NO: 36 - DNA, 21 bp, Artificial sequence
  Synthetic oligonucleotide
ctctccacacatggctccttt

SEQ ID NO: 37 - DNA, 21 bp, Artificial sequence
  Synthetic oligonucleotide
cctacgaacaggtgatgcact

SEQ ID NO: 38 - DNA, 21 bp, Artificial sequence
  Synthetic oligonucleotide
gatttcttctcccttgcgtct

SEQ ID NO: 39 - DNA, 21 bp, Artificial sequence
  Synthetic oligonucleotide
ctcagttcctacgcttcgcat

SEQ ID NO: 40 - DNA, 20 bp, Artificial sequence
  Synthetic oligonucleotide
gtcgaggtcagtgaacagca

SEQ ID NO: 41 - DNA, 20 bp, Artificial sequence
  Synthetic oligonucleotide
gcacctctcccagcatctca

SEQ ID NO: 42 - DNA, 20 bp, Artificial sequence
  Synthetic oligonucleotide
gtcggaggatctctcgttgc

SEQ ID NO: 43 - DNA, 22 bp, Artificial sequence
  Synthetic oligonucleotide
aaaattacacgccagatttgcc

SEQ ID NO: 44 - DNA, 23 bp, Artificial sequence
  Synthetic oligonucleotide
ggtgtgacattactccagagttg

SEQ ID NO: 45 - DNA, 19 bp, Artificial sequence
  Synthetic oligonucleotide
aagtagccatggcgctgta

SEQ ID NO: 46 - DNA, 20 bp, Artificial sequence
  Synthetic oligonucleotide
gagacgtgactgctgtggag

SEQ ID NO: 47 - DNA, 21 bp, Artificial sequence
  Synthetic oligonucleotide
ctgaggatctctggttgtggt

SEQ ID NO: 48 - DNA, 22 bp, Artificial sequence
  Synthetic oligonucleotide
cgaactggacacacatacagtg

SEQ ID NO: 49 - skipped

SEQ ID NO: 50 - DNA, 18 bp, Artificial sequence
  Synthetic oligonucleotide
cgggagtccgcagtctta

SEQ ID NO: 51 - DNA, 20 bp, Artificial sequence
  Synthetic oligonucleotide
gcttgagggtctgaatcttg

SEQ ID NO: 52 - DNA, 20 bp, Artificial sequence
  Synthetic oligonucleotide
cttatgtttggggggaggtt

SEQ ID NO: 53 - DNA, 20 bp, Artificial sequence
  Synthetic oligonucleotide
tagccaagcaatcacggaga

SEQ ID NO: 54 - DNA, 21 bp, Artificial sequence
  Synthetic oligonucleotide
ctttgtcgttggttagctggt

SEQ ID NO: 55 - DNA, 20 bp, Artificial sequence
  Synthetic oligonucleotide
gacgccatcaacaccgagtt

SEQ ID NO: 56 - DNA, 22 bp, Artificial sequence
  Synthetic oligonucleotide
atcagcctaaccatacaactct

SEQ ID NO: 57 - DNA, 19 bp, Artificial sequence
  Synthetic oligonucleotide
caggaacaaattggcacaa

SEQ ID NO: 58 - DNA, 22 bp, Artificial sequence
  Synthetic oligonucleotide
gggttctttcatttgttttggt

SEQ ID NO: 59 - DNA, 23 bp, Artificial sequence
  Synthetic oligonucleotide
actccaaacagcttctcttctga

SEQ ID NO: 60 - DNA, 21 bp, Artificial sequence
  Synthetic oligonucleotide
cagatcacgtcatcgcacaac

SEQ ID NO: 61 - DNA, 21 bp, Artificial sequence
  Synthetic oligonucleotide
gagggccaagacgaagacatc

SEQ ID NO: 62 - DNA, 19 bp, Artificial sequence
  Synthetic oligonucleotide
gcccggatgtcgcttacag

SEQ ID NO: 63 - DNA, 21 bp, Artificial sequence
  Synthetic oligonucleotide
aaatgcagacgcagggtacag

SEQ ID NO: 64 - DNA, 20 bp, Artificial sequence
  Synthetic oligonucleotide
gactccaccgagatcgacca

SEQ ID NO: 65 - DNA, 21 bp, Artificial sequence
  Synthetic oligonucleotide
cttgtagcactctccgtaggc

SEQ ID NO: 66 - DNA, 22 bp, Artificial sequence
  Synthetic oligonucleotide
gcacagaatccttggtgaacag

SEQ ID NO: 67 - DNA, 22 bp, Artificial sequence
  Synthetic oligonucleotide
atggaaggtgaatgtttcagca

SEQ ID NO: 68 - DNA, 21 bp, Artificial sequence
  Synthetic oligonucleotide
cagtggaccccatcatggaga

SEQ ID NO: 69 - DNA, 21 bp, Artificial sequence
  Synthetic oligonucleotide
ggctttgctgccttgaacatc

SEQ ID NO: 70 - DNA, 21 bp, Artificial sequence
  Synthetic oligonucleotide
cttggggccaaaaaggtgttc

SEQ ID NO: 71 - DNA, 21 bp, Artificial sequence
  Synthetic oligonucleotide
gaggtagcggtcctcatacag

SEQ ID NO: 72 - DNA, 22 bp, Artificial sequence
  Synthetic oligonucleotide
aatgaggtgggcttaaagcaag

SEQ ID NO: 73 - DNA, 21 bp, Artificial sequence
  Synthetic oligonucleotide
agttccactccacagttcaga

SEQ ID NO: 74 - DNA, 20 bp, Artificial sequence
  Synthetic oligonucleotide
cggcccgtgtattgtctagc

SEQ ID NO: 75 - DNA, 21 bp, Artificial sequence
  Synthetic oligonucleotide
tagccagggtgtccaaatctg

SEQ ID NO: 76 - DNA, 21 bp, Artificial sequence
  Synthetic oligonucleotide
ggtggtgaatgaaacgctcag

SEQ ID NO: 77 - DNA, 21 bp, Artificial sequence
  Synthetic oligonucleotide
acccctttgggaatgaacatc

SEQ ID NO: 78 - DNA, 20 bp, Artificial sequence
  Synthetic oligonucleotide
cactggtcgccatctttgta

SEQ ID NO: 79 - DNA, 20 bp, Artificial sequence
  Synthetic oligonucleotide
agtgcatggaagagacctgc

SEQ ID NO: 80 - DNA, 20 bp, Artificial sequence
  Synthetic oligonucleotide
acagggctctgaacatgcac

SEQ ID NO: 81 - DNA, 20 bp, Artificial sequence
  Synthetic oligonucleotide
ggcattgaaaaactcccgta

SEQ ID NO: 82 - DNA, 21 bp, Artificial sequence
  Synthetic oligonucleotide
actacatcaacgaccgccagt

SEQ ID NO: 83 - DNA, 21 bp, Artificial sequence
  Synthetic oligonucleotide
atctgctcgatcatctgccag

(19) United States
(12) Patent Application Publication
(10) Pub. No.: US 2024/0182867 A1
(43) Pub. Date: Jun. 6, 2024

(54) CHEMICAL REPROGRAMMING OF HUMAN SOMATIC CELLS INTO PLURIPOTENT CELLS

(71) Applicant: Peking University, Beijing (CN)

(72) Inventors: Hongkui Deng, Beijing (CN); Jingyang Guan, Beijing (CN); Jinlin Wang, Beijing (CN); Guan Wang, Beijing (CN); Zhengyuan Zhang, Beijing (CN); Yao Fu, Beijing (CN); Lin Cheng, Beijing (CN); Gaofan Meng, Beijing (CN)

(21) Appl. No.: 18/285,494

(22) PCT Filed: Feb. 21, 2022
(86) PCT No.: PCT/CN2022/077048

§ 371 Date: Oct. 3, 2023

(30) Foreign Application Priority Data
Apr. 8, 2021 (WO) PCT/CN2021/085936

(51) Int. Cl. C12N 5/074

(52) U.S. Cl. CPC C12N 5/0696; C12N 2501/115; C12N 2501/117; C12N 2501/15; C12N 2501/155; C12N 2501/16; C12N 2501/385; C12N 2501/727; C12N 2506/1307

(57) ABSTRACT

Compositions of culture conditions and the stepwise method for improving reprograming of human somatic cells into human chemically induced pluripotent cells are disclosed. The first stage, which uses a combination of small molecules with necessary biological activities, is aimed at downregulating the somatic gene program. The second stage uses a selection of small molecules with select biological activities to upregulate one or more pluripotency-related transcriptional factors. The third stage uses a selection of small molecule factors with select biological activities to establish an initial pluripotency network, measured by the expression of OCT4. The fourth and final stage uses a selection of small molecules with select biological activities to fully establish a pluripotent network, measured by co-expression factors such as OCT4, SOX2, and NANOG in the reprogrammed cells. The resultant reprogrammed cells are termed human chemically induced pluripotent stem cells, hCiPSCs.

### FIELD OF THE INVENTION

This invention is generally in the field of chemical reprogramming of somatic cells into cells with characteristics of pluripotent stem cells.

### BACKGROUND OF THE INVENTION

Cell identity is established, maintained, and changed in response to external signals from the microenvironment during the states of development, homeostasis, and disease (1, 2). In plants and some invertebrates, external stimulation sufficiently triggers cell dedifferentiation and regeneration (3, 4). However, using only external perturbation to restart the plasticity potentials of mammalian cells, especially human somatic cells, is a daunting challenge because of the stable epigenetic landscape that protects committed cell identity (5, 6). Previous studies using small molecules as an external chemical perturbation to induce mouse somatic cells to pluripotent stem cells, demonstrated a chemical toward cell fate reprogramming (7-10). As a highly tunable way to control cell fates, the small molecule combinations can manipulate cell fates by regulating multiple cell-signaling pathways and chromatin states (11-12). However, efficient and robust reprogramming of human somatic cells still requires improvement, because of a more stable human epigenome and a reduced plasticity that are developed along evolution (5-6, 13-14).

It is an object of the present invention to provide a combination of small molecules which can be used to reprogram human somatic cells into pluripotent cells.

It is also an object of the present invention to provide an improved method of reprogramming human somatic cells into pluripotent cells with improved efficiency.

### SUMMARY OF THE INVENTION

Compositions and methods are disclosed for improving human somatic cells into pluripotent cells. The disclosed methods overcome the inadequacy of prior art methods by adopting a four stage reprogramming approach, which selectively inhibit/activate a combination of biological activities in the somatic cell, allowing for improved reprogramming of human somatic cells. The first stage uses a combination of small molecules with required biological activities (Stage I factors) aimed at downregulating the somatic gene program. The second stage uses a selection of small molecules with select biological activities (Stage II factors) to upregulate one or more pluripotency-related transcriptional factors. The third stage uses a selection of small molecule factors with select biological activities (Stage III factors) to establish an initial pluripotency network, measured by the expression of OCT4. The fourth and final stage uses a selection of small molecules with select biological activities (Stage IV factors) to fully establish a pluripotent network, measured by co-expression factors such as OCT4, SOX2, and NANOG in the reprogrammed cells (herein, termed human chemically induced pluripotent stem cells, hCiPSCs).

A preferred combination of Stage I factors selected from small molecules with the following biological activities: a glycogen kinase inhibitor, for example, CHIR99021, a TGFβ inhibitor, for example, 616452, and a retinoic acid receptor (RAR) agonist, for example, TTNPB, are used in stage I to convert human somatic cells for example, human fibroblasts into a monolayer epithelial-like cells, by culturing the cells in cell culture medium supplemented with Stage I factors for an effective amount of time to convert the cells into a monolayer epithelial-like cells (Stage I Condition). In some preferred embodiments, small molecules with one or more of the following biological activities can be included in the Stage I condition: a selective inhibitor of Rho-associated, coiled-coil containing protein kinase (ROCK), for example, Y27632; a receptor tyrosine kinase inhibitor, for example, ABT869; an agonist for the G protein-coupled receptor Smoothened, for example, SAG; a Dot1L inhibitor, for example, EPZ004777 or EPZ5676; a Jak 1/Jak2 inhibitor, for example, Ruxolitinib, an SAH hydrolase inhibitor for example, DZNep, and a Menin-MLL interaction inhibitor, for example, VTP50469, MI3454, or WDR5-IN-4 (Stage I supplemental factors).

A preferred combination of Stage II factors selected from small molecules with the following biological activities: a glycogen kinase inhibitor, for example, CHIR99021, a TGFß inhibitor, for example, 616452, a retinoic acid receptor (RAR) agonist, for example, TTNPB, an agonist for the G protein-coupled receptor Smoothened, for example, SAG, and a c-Jun kinase inhibitor for example, JNKIN8 are used in stage II to upregulate one or more pluripotency-related transcriptional factors, by culturing the cells in cell culture medium supplemented with Stage II factors for an effective amount of time to upregulate one or more pluripotency-related transcriptional factors (Stage II Condition). In some preferred embodiments, small molecules with one or more of the following biological activities can be included in the Stage II condition: a DNA methyltransferase inhibitor, for example, 5-Azacytidine, inhibitor of histone demethylation, for example, Tranylcypromine, a selective inhibitor of Rho-associated, coiled-coil containing protein kinase (ROCK), for example, Y27632, a receptor tyrosine kinase inhibitor, for example, ABT869, a G9a inhibitor, for example, UNC0224, a BMP receptors/AMPK inhibitor, for example, Dorsomorphin, a Jak1/Jak2 inhibitor, for example, Ruxolitinib, a p38 MAPK inhibitor, for example, BIRB796, a CBP/p300 bromodomain inhibitor, for example, SGC-CBP30, I-CBP112, GNE272, or GNE409, and a Menin-MLL interaction inhibitor, for example, VTP50469, MI3454, or WDR5-IN-4 (Stage II supplemental factors).

A preferred combination of Stage III factors selected from small molecules with the following biological activities: a histone deacetylase inhibitor, for example, Valproic acid, a MAPK inhibitor, for example, PD0325901, a TGFβ inhibitor, for example 616452, and an SAH hydrolase inhibitor for example, DZNep are used in stage three to establish an initial pluripotency network, e.g., measured by the expression of OCT4, by culturing the cells in cell culture medium supplemented with Stage III factors for an effective amount of time to establish an initial pluripotency network (Stage III Condition). In some preferred embodiments, small molecules with one or more of the following biological activities can be included in the Stage III condition: a glycogen kinase inhibitor, for example, CHIR99021, a selective inhibitor of Rho-associated, coiled-coil containing protein kinase (ROCK), for example, Y27632, a SETD8 inhibitor, for example, Unc0379, inhibitor of histone demethylation, for example, Tranylcypromine, and a Dot1L inhibitor, for example, EPZ004777 (Stage III supplemental factors).

A preferred combination of Stage IV factors selected from small molecules with the following biological activities: a B-Raf inhibitor, for example, SB590885, and a MAPK inhibitor, for example, PD0325901 are used in stage four to fully establish a pluripotent network, e.g., measured by co-expression OCT4, SOX2, and NANOG, by culturing the cells in cell culture medium supplemented with Stage IV factors for an effective amount of time to fully establish a pluripotent network (Stage IV Condition). In some preferred embodiments, small molecules with one or more of the following biological activities can be included in the Stage IV condition: a Wnt inhibitor, for example, IWP2, a glycogen kinase inhibitor, for example, CHIR99021 or CHIR98014, a selective inhibitor of Rho-associated, coiled-coil containing protein kinase (ROCK), for example, Y27632, and a histone deacetylase inhibitor, for example, Valproic acid (Stage IV supplemental factors).

The disclosed method can be used to reprogram human somatic cells that isolated from fetal or adult donor tissues, such as human embryonic fibroblasts, adult human skin dermal fibroblasts and adult adipose-derived mesenchymal stromal cells.

Cells (e.g., hCiPSCs) obtained according to the disclosed method, are also provided. The cells obtained according to the disclosed method include, for example, (i) epithelia-like cells obtained by culture of stage I, characterized in down-regulation of at least one gene at early stage MMP1, ZEB1, VIM, COL1A1, COL5A1, COL6A2, PRRX1, SNAI2, TWIST1, and TWIST2, and up-regulation of at least one gene relating to LIN28A and KRT, e.g., KRT8, KRT18, KRT19, and LIN28A; (ii) plasticity state cells with regeneration program obtained by culture of stage I and stage II, characterized in that they express at least one of SALL4 and LIN28A, and unlocked epigenome state with increased number of opened chromatin loci, and increased DNA demethylation; (iii) XEN-like cells obtained by culture of stage I, stage II and stage III, characterized in up-regulation of at least one gene LIN28A, SALL4, and OCT4, and expression of at least one of XEN (extraembryonic endoderm) related markers, e.g., GATA6, SOX17, FOXA2, HNF1B, APOA1, and APOA2; and/or (iv) human chemically induced pluripotent stem cells, characterized in that they express at least one surface marker TRA-1-60, TRA-1-81, and SSEA-4, along with the core pluripotency transcriptional factors OCT4, SOX2, DNMT3B, DPPA4, UTFI, ZFP42, ZIC3, and NANOG. The hCiPSCs are characterized in that they can expand for more than 20 passages, for example, for up to 25, 30, 35, 40, 41, 42, passages, proliferate with a doubling time similar to that of hESCs. hCiPSCs are also characterized in that they express at least one surface marker TRA-1-60, TRA-1-81, and SSEA-4, along with the core pluripotency transcriptional factors OCT4, SOX2, DNMT3B, DPPA4, UTF1, ZFP42, ZIC3, and NANOG. In a preferred embodiment, they express TRA-1-60, TRA-1-81, and SSEA-4. The primary hCiPSCs which induced at the end of stage IV express several unique markers, such as Developmental Pluripotency Associated 3 (DPPA3), Kruppel-Like Factor 17 (KLF17) and DNA methyltransferase 3 like (DNMT3L). These markers were not expressed in the traditional human pluripotent stem cells (hESCs and hiPSCs). Functionally, when injected hCiPSCs into immunodeficient mice and the resultant teratomas contain tissues of all 3 germ layers (endo-, ecto-, and mesoderm); hCiPSCs form embryoid bodies in vitro and expressed marker genes of the three germ layers and can be subjected to directed differentiation into another committed cell type, for example, hepatocytes, or directed differentiation into progenitor cells such as hematopoietic progenitor cells. Most importantly, hCiPSCs are preferably, not genetically engineered, i.e., not obtained by a process that includes altering human somatic cells by introducing or removing genetic elements from the cells, for example engineering somatic cells to express one or more markers of pluripotency such as OCT4, SOX2, KLF4, NANOG and/or c-Myc and accordingly, hCiPSCs obtained following the disclosed methods preferably do not contain exogenously introduced OCT4, SOX2, KLF4, NANOG and/or c-Myc.

A cell culture media composition or kit for reprogramming human somatic cells into human chemically induced pluripotent cells (e.g., for use in the disclosed method) is also provided. The composition or kit may include a cocktail of a combination of the molecules of one or more, preferably all, of Stages I-IV disclosed herein. These may be in a form having defined concentrations to facilitate addition to cell culture media to produce a desired concentration. The composition or kit may be used in preparing cells of one or more of Stages I-IV disclosed herein.

Accordingly, compositions of culture conditions and the stepwise method for improving reprograming of human somatic cells into human chemically induced pluripotent cells are disclosed. The first stage, which uses a combination of small molecules with necessary biological activities (Stage I), is aimed at downregulating the somatic gene program. The second stage uses a selection of small molecules with select biological activities (Stage II) to upregulate one or more pluripotency-related transcriptional factors. The third stage uses a selection of small molecule factors with select biological activities (Stage III) to establish an initial pluripotency network, measured by the expression of OCT4. The fourth and final stage uses a selection of small molecules with select biological activities (Stage IV) to fully establish a pluripotent network, measured by co-expression factors such as OCT4, SOX2, and NANOG in the reprogrammed cells. The resultant reprogrammed cells are termed human chemically induced pluripotent stem cells, hCiPSCs.

In addition, the compositions of culture conditions and the stepwise method disclosed here can generate a source of stem cells, progenitor cells, dedifferentiated cells or cells with plasticity potentials that have the ability to give rise to a desired cell type, and is important for therapeutic treatments, tissue engineering and research. The cells obtained through the methods described in this document including the hCiPSCs, the XEN-like cells, plasticity state cells, and epithelia-like cells are readily available sources of stem cells, progenitor cells, dedifferentiated cells or cells with plasticity potentials which express at least one stem cell or progenitor cell related marker such as LIN28A, SALL4, OCT4 or NANOG. Although in this document, for the sake of brevity, the source of stem cells, progenitor cells, dedifferentiated cells or cells with plasticity potentials was designated hCiPSCs, one skilled in the art would understand that the XEN-like cells, plasticity state cells, and epithelia-like cells obtained by methods described in this document may also be used similarly as a source of stem cells, progenitor cells, dedifferentiated cells or cells with plasticity potentials.

Furthermore, the small molecule compositions in this application can be used for tissue regeneration, repair and rejuvenation in vitro and in vivo. For example, small molecules for Stage I and Stage II of the reprogramming process can be formulated for administration, delivery or contacting with a subject, tissue or cell to promote de-differentiation, regeneration, repair and rejuvenation in vivo or in vitro/ex vivo.

### BRIEF DESCRIPTION OF THE DRAWINGS

FIGS. 1A-D and 1J show generation of hCiPSCs by small molecule compounds. FIG. 1A schematic diagram of the chemical reprogramming process from fibroblasts to hCiPSCs. FIG. 1B shows morphological changes of human embryonic fibroblasts after treatment of mouse chemical reprogramming condition. Scale Bar, 100 μm. Representative of at least three independent experiments. FIG. 1C shows morphology of untreated HEFs. FIG. 1D shows morphology of hCiPSCs derived from HEFs (hCiPSCs-1117-1#-p21). FIG. 1E shows representative images of cells at the end of each stage during hCiPSCs induction. Scale bar, 100 μm. Representative of at least three independent experiments. FIGS. 1F-I show quantitative PCR validations of gene expressions during hCiPSC induction. FIG. 1F and 1G show relative expression of fibroblast-related genes, epithelial-related genes, and LIN28A at the end of stage I. FIG. 1H shows relative expression of LIN28A and SALL4 at the end of stage II. FIG. 1I shows relative expression of LIN28A, SALL4, and OCT4 at the end of stage III. Error bars, mean (SD). Representative of three independent experiments. FIG. 1J RT-qPCR analysis of pluripotency markers in the indicated hCiPSCs and hESCs. Scale bars, 100 μm. Error bars, mean (SD); n=3.

FIG. 2A-2E show generation of hCiPSCs from adult somatic cells. FIG. 2A. Representative images of adult hADSCs and hADSC-derived hCiPSCs. FIG. 2B. RT-qPCR analysis of pluripotency markers in the indicated hCiPSCs and hESCs. FIG. 2C. Hierarchical clustering of global transcriptomic profiles of hCiPSCs, hESCs, and adult somatic cells. FIG. 2D. Bisulfite sequencing analysis of the DNA CpG methylation statuses in hADSCs' and hADSC-derived hCiPSCs' (hCiPSCs-0809-3#) OCT4 and NANOG promoter loci. FIG. 2E. Hematoxylin and eosin staining of hCiPSC-derived teratoma sections (hCiPSCs-0809-3# and hCiPSCs-1003#). For each hCiPSC clone, all the images were obtained from a single teratoma that contained representative tissue features of ectoderm, mesoderm, and endoderm.

FIG. 3A shows gene ontology analyses of genes downregulated in stage I and upregulated in stage II. Four batches of independent experiments using HEFs and hADSCs were used to analyze the overlapping differentially expressed genes. FIG. 3B. Heat map showing the opened genes that detected by sc-ATAC-seq in fibroblasts and stage II. FIG. 3C. Distribution of the different DNA CpG methylation statuses in fibroblasts, stage I and stage II. Representative of three independent experiments. FIG. 3D. Venn diagrams showing the number of methylated gene promoters in fibroblasts, stage I and stage II. FIG. 3E. The gene expression enrichment, chromatin accessibility and DNA methylation status of the genes in the indicated GO terms.

FIG. 4A-F show data of the key small molecules that regulated plasticity signatures at the early stages. FIG. 4A and 4B. Expression of LIN28A and fibroblast marker genes at the end of stage I and the numbers of hCiPSC colonies at the end of reprogramming after withdrawing individual chemicals from stage I. Representative of three independent experiments. FIG. 4C and 4D. The number of SALL4-positive colonies at stage II and hCiPSC colonies at the end of reprogramming after withdrawing individual chemicals from stage II. Representative of three independent experiments. FIG. 4E. Heat map showing the indicated gene expression for the indicated samples. FIG. 4F. The signaling density of the opened loci in stage II after withdrawing individual chemicals. FIG. 4G. DNA methylation levels after withdrawing individual chemicals from stage II. FIG. 4H. The gene expression enrichment, chromatin accessibility and DNA methylation status of the genes in the indicated GO terms after withdrawing individual chemicals from stage II. FIG. 4I. Schematic diagram for key molecular events during the reprogramming process at the early stages. Error bars, mean (SD); n=3.

FIG. 5A shows characterization of hCiPSCs exemplified as the calculated doubling time of hCiPSCs and hESCs. Error bars, mean (SD). FIGS. 5B and C show global gene expression analysis in hCiPSCs. FIG. 5B.Scatter plots comparing the global gene expression in hCiPSCs, hESCs and HEFs. Representative of three independent experiments. FIG. 5C. Hierarchical clustering of global transcriptional profiles of hCiPSCs, hESCs and HEFs. Distance was calculated by I-spearman correlation coefficient. FIG. 5D shows the Expression of Kruppel-Like Factor 17 (KLF17), Developmental Pluripotency Associated 3 (DPPA3), and DNA methyltransferase 3 like (DNMT3L) in the fibroblasts, primary hCiPSCs, and human embryonic stem cells (hESCs).

FIGS. 6A-6C show in vitro directed differentiation of hCiPSCs to hematopoietic lineage cells. FIGS. 6A and 6B. The percentages of CD34/CD144 double-positive and CD34/CD31 double-positive cells counted by FACS analysis. Error bars, mean (SD); n=3. FIG. 6C. The percentage of hCiPSC-derived T progenitor cells counted by FACS analysis.

FIGS. 7A-D show in vitro directed differentiation of hCiPSCs to hepatocytes. FIG. 7A. FACS analysis of AFP and ALB double-positive hCiPSC-derived hepatic progenitor cells (HPCs). FIG. 7B. hCiPSC-derived hepatocyte (hdHeps) morphology. Scale bar, 100/lm. FIG. 7C. Quantitative analysis of urea and ALB secretion by hdHeps and primary human hepatocytes (PHHs). FIG. 7D. RT-qPCR analyses of functional hepatocyte marker genes in hiPSCs, HPCs, hdHeps, and PHHs. Relative expression was normalized to PHHs. Error bars, mean (SD); n=3.

FIG. 8 shows karyotype analysis showing REF-derived hCiPSCs with a normal, diploid chromosomal content.

FIG. 9A-D show results for small molecules that promote reprogramming efficiency of hADSCs. FIG. 9A. Representative images of cells at the end of each stage during hCiPSC induction from hADSCs. Scale bar, 100 flm. FIG. 9B and 9C Identification of the small molecules and their combinations that promote the generation of hCiPSCs from hADSCs at stage I and stage II. EPZ, EPZ004777; Ruxo, Ruxolitinib; BIRB, BIRB796; U2, UNC0224; DM, Dorsomorphin. Error bars, mean (SD); n=3. FIG. 9D. Numbers of hCiPSC colonies generated from hADSCs under different time courses. “8+8+8+7” represents a sequential duration for each stage, respectively. Error bars, mean (SD); n=4. All the results above are representative of at least three independent experiments.

FIG. 10A-B show characterization of hCiPSCs derived from hADSCs. FIG. 10A. Bisulfite genomic sequencing of the OCT4 and NANOG promoter regions in hCiPSCs, hESCs and hADSCs. FIG. 10B. Karyotype analysis showing normal, diploid chromosomal content for hADSC-derived hCiPSCs.

FIG. 11A-C show characterization of hCiPSCs derived from human adult skin fibroblasts (hASFs). FIG. 11A. Images of representative adult hASFs and hASF-derived hCiPSCs. FIG. 11B. Real-time qPCR analyses of pluripotency markers in the indicated hCiPSCs and hESCs; the bars in each group of 6 bars are labelled from left to right as follows: 1=hCiPSCs-0408-1#; 2=hCiPSCs-0408-2#; 3=hCiPSCs-1230-4#; 4=hCiPSCs-1230-11#; 5=H1; 6=H9). FIG. 11C. Karyotype analysis showing normal, diploid chromosomal content of hASF-derived hCiPSCs.

FIG. 12A-B show XEN-like state were induced in stage III. FIG. 12A. Dynamic expression change of XEN-related markers during the chemical reprogramming process, as shown by RT-qPCR. Error bars, mean (SD); n=3. Representative of three independent experiments. FIG. 12B. Percentages of OCT4-positive cells generated from the inside (black) and outside (red) the XEN-like colonies in 5 batches of independent experiments. More than 3 wells were counted for each experimental batch.

FIG. 13A-C show single-cell RNA sequencing analysis of the cells at the end of stage III. FIG. 13A. Expression of primitive endoderm (also known as XEN) related markers in stage III. FIG. 13B. Expression of primitive endoderm related genes in human pre-implantation embryo and the cell clusters in stage III. FIG. 13C. GSEA analysis of the enrichment score of primitive endoderm related features in the XEN-like cells (cluster 2).

FIG. 14 is a schematic diagram for key molecular events during the reprogramming process.

FIG. 15A-C show the effects of withdrawing individual chemicals from stage I conditions. (A) Hierarchical clustering of global transcriptional profiles of cells after the indicated treatments in stage I. (B) and (C) Gene ontology analyses of the differently expressed genes after remove of CHIR, 616452 and TTNPB.

FIG. 16A-C show the effects of withdrawing individual chemicals from the stage II condition. (A and B) Expression of SALL4 and cell numbers after withdrawing indicated chemicals at the end of stage II. Error bars, Mean with SD (n=3). ****p <0.0001. Representative of three independent experiments. (C) Gene ontology analyses of the differently expressed genes after removal of JNKIN8.

FIG. 17 shows the reprogramming efficiency after removing individual small molecules from stage I, Stage II, Stage III and Stage IV, and the core small molecules can be identified by these data.

FIG. 18 shows the reprogramming efficiency after the small molecules in stage I were replaced by other small molecules that targeting the same pathway or targets (GSK3β inhibitor; RA pathway agonists; Rock inhibitors; TGFβ inhibitors).

FIG. 19 shows the reprogramming efficiency after the small molecules in stage II were replaced by other small molecules that targeting the same pathway or targets (GSK3β inhibitor; RA pathway agonists; Rock inhibitors; TGFβ inhibitors).

FIG. 20 shows the reprogramming efficiency after the small molecules in stage II were replaced by other small molecules that targeting the same pathway or targets (Smoothened agonist; histone demethylation inhibitor; DNMT inhibitor; JNK inhibitor).

FIG. 21 shows the reprogramming efficiency after the small molecules in stage III were replaced by other small molecules that targeting the same pathway or targets (GSK3β inhibitors; TGFβ inhibitors; Rock inhibitors; histone demethylation inhibitors and Rock inhibitors).

FIG. 22 shows the reprogramming efficiency after the small molecules in stage III were replaced by other small molecules that targeting the same pathway or targets (HDAC inhibitors; Dot1L inhibitors; S-adenosyl-L-homocysteine hydrolase inhibitors; ERK inhibitors).

FIG. 23 shows the reprogramming efficiency after the small molecules in stage IV were replaced by other small molecules that targeting the same pathway or targets (GSK3β inhibitors; ERK inhibitors; Rock inhibitors; WNT pathway inhibitors)

FIG. 24 shows the reprogramming efficiency after the small molecules in stage IV were replaced by other small molecules that targeting the same pathway or targets (BRAF inhibitors and HDAC inhibitors).

FIG. 25 illustrates the induction process of hCiPSCs by small molecule compounds.

FIG. 26 shows the CBP/p300 inhibitors including SGC-CBP30, I-CBP112, GNE272 and GNE409 can improve the reprogramming efficiency.

FIG. 27 shows the Menin-MLL interaction inhibitor can improve the reprogramming efficiency.

FIG. 28 shows the SETD8 inhibitor can improve the reprogramming efficiency.

FIG. 29 shows minimal chemical combinations and the small molecules that improve the reprogramming efficiency in stage I.

FIG. 30 shows minimal chemical combinations and the small molecules that improve the reprogramming efficiency in stage II.

FIG. 31 shows minimal chemical combinations and the small molecules that improve the reprogramming efficiency in stage III.

FIG. 32 shows minimal chemical combinations and the small molecules that improve the reprogramming efficiency in stage IV.

### DETAILED DESCRIPTION OF THE INVENTION

External stimulation sufficiently triggers cell dedifferentiation and regeneration in plants and some invertebrates. However, because of their committed cell identities and stable epigenomes, human somatic cells resist external perturbations to modulate their plasticity to unrestricted potency. Here, small molecule combinations with select biological activities as an external chemical perturbation allowed for effective reprogramming of the epigenetic landscape of differentiated human somatic cells, via a dedifferentiation process resembling natural regeneration. This chemical reprogramming of somatic cells resulted in human chemically induced pluripotent stem cells (hCiPSCs) exhibiting key features of pluripotent stem cells in transcriptomic profiles, epigenetic status and developmental potential. The disclosed approach provides a platform to generate human pluripotent stem cells for applications. Furthermore, the studies disclosed herein also sheds light on therapeutic reprogramming wherein the plasticity potential of human somatic cells can be modulated by external stimuli both in vitro and in vivo.

### I. Definitions

The term “chemically induced pluripotent stem cells” (CiPSCs) as used herein refers to pluripotent cells derived from a cell that is not pluripotent, i.e., a multipotent or differentiated cells, by contacting the non-pluripotent cell with chemical compounds, not by expression of one or more transfected genes.

As used herein a “culture” means a population of cells grown in a medium and optionally passaged. A cell culture may be a primary culture (e.g., a culture that has not been passaged) or may be a secondary or subsequent culture (e.g., a population of cells which have been subcultured or passaged one or more times).

As used herein “enhancing”, or “increasing” the efficiency of reprogramming means reducing total reprograming time, increasing the number of reprogrammed cells obtained from the same starting cell density cultured for the same length of time and/or improving the quality of reprogrammed cells, measured in terms of characteristics selected from the ability of the cells to express pluripotency factors such as OCT4, SOX2 and NANOG and number of passages in culture, when compared to a chemical reprograming method that does not use the enhancing factor (small molecule).

The term “isolated” or “purified” when referring to CiPSCs means chemically induced pluripotent stem cells at least 30%, 35%, 40%, 45%, 50%, 55%, 60%, 65%, 70%, 75%, 80%, 85%, 90%, 95%, or 99% free of contaminating cell types such as non-pluripotent cells. The isolated stem cells may also be substantially free of soluble, naturally occurring molecules.

The term “pluripotency” (or pluripotent), as used herein refers the potential of a cell to differentiate into any of the three germ layers: endoderm (for example, interior stomach lining, gastrointestinal tract, the lungs), mesoderm (for example, muscle, bone, blood, urogenital), or ectoderm (for example, epidermal tissues and nervous system). The term “not pluripotent” means that the cell does not have the potential to differentiate into all of the three germ layers. A multipotent stem cell is less plastic and more differentiated, and can become one of several types of cells within a given organ. For example, multipotent blood stem cells can develop into red blood cell progenitors, white blood cells or platelet producing cells. Adult stem cells are multipotent stem cells. Adipose-derived stem cells are multipotent.

“Reprogramming” as used herein refers to the conversion of a one specific cell type to another. For example, a human somatic cell that is not pluripotent can be reprogrammed into a pluripotent cell. Where the non-pluripotent cell is reprogrammed into a pluripotent cell using chemical compounds, the resulting cell is a chemically induced pluripotent stem cell.

The term “small molecule” refers to a molecule, such as an organic or organometallic compound, with a molecular weight of less than 2,000 Daltons, more preferably less than 1,500 Daltons, and most preferably less than 1,000 Daltons.

The term “dedifferentiated” refers to a cell or tissue which has developed in reverse, from a more differentiated to a less differentiated state. The cells or tissue acquired a less mature appearance, gene expression, epigenetic state or metabolic profile.

The term “plasticity” refers to the ability of cells or tissues to take on the characteristics of other cells or tissues. The cell plasticity indicates that the cell has considerable potential to overcome cross-lineage restriction boundary and give rise to other cell types.

### II. Compositions

### A. Small Molecules for Improving Induction of Pluripotency in Human Somatic Cells

Chemical compounds that are useful for improving reprograming of human somatic cells into human chemically induced pluripotent cells include small molecules having a molecular weight of less than 2,000 Daltons, more preferably less than 1,500 Daltons, and most preferably less than 1,000 Dalton, alone or in combination with proteins. The small molecules may have a molecular weight less than or equal to 900 Daltons or, less than or equal to 500 Daltons.

Accordingly, small molecule cocktails have been identified which inhibit/activate a combination of select cellular activities, improving reprogramming of human somatic cells into human chemically induced pluripotent cells (without a need to genetically engineer the cells to express one or more markers of pluripotency such as Oct4) in a four stage process of chemical reprograming. Improved reprogramming of human somatic cells by selective inhibition./activation of the disclosed biological activities can be determined for example, as improved quality of reprogrammed cells, measured in terms of characteristics selected from the ability of the cells to express pluripotency factors such as OCT4, SOX2 and NANOG, alone, or in combination with at least one surface marker TRA-1-60, TRA-1-81, and SSEA-4, and/or number of passages (of the reprogrammed cells) in culture, when compared to a chemical reprograming method that does not use the combination of inhibition/activation of the biological activities disclosed herein.

Small molecules for Stage I of the reprogramming process include a combination of small molecules (herein, Stage I condition) with biological activities selected from: a glycogen kinase inhibitor, with CHIR99021, being used in preferred embodiments, a TGFβ inhibitor, with 616452 being used in preferred embodiments, and a retinoic acid receptor (RAR) agonist, with TTNPB being used in preferred embodiments. In some embodiments, additional small molecules with select biological activities are included in the Stage I condition, for example, an inhibitor of Rho-associated, coiled-coil containing protein kinase (ROCK), with Y27632 or Tzv being used in the preferred embodiments, a receptor tyrosine kinase inhibitor, with ABT869 being used in preferred embodiments and an agonist for the receptor Smoothened, with SAG being used as a preferred embodiments.

The Stage I condition is used to convert human somatic cells for example, human fibroblasts, adipose-derived stromal cells, etc., into monolayer epithelial-like cells and can optionally be supplemented with stage I supplemental biological activities such as an SAH hydrolase inhibitor with DZNep being used in the preferred embodiments, a DOT1L methyltransferase inhibitor with EPZ004777 used in preferred embodiments, and a JAK1/JAK2 inhibitor, with Ruxolitinib being used in preferred embodiments.

Small molecules for Stage II of the reprogramming process include a combination of small molecules with select biological activities (herein, Stage II condition) (i) the Stage I factors: a glycogen kinase inhibitor, with CHIR99021, being used in preferred embodiments, a TGFβ inhibitor, with 616452 being used in preferred embodiments, a selective inhibitor of Rho-associated, coiled-coil containing protein kinase (ROCK), with Y27632 being used in the preferred embodiments and an agonist for the receptor Smoothened, with SAG being used in preferred embodiments (although Purmorphamine; Hh-Ag1.5; SAG 21K or human SHH can be used as the agonist for the receptor Smoothened) and a receptor tyrosine kinase inhibitor, with ABT869 being used in the preferred embodiments; (ii) the Stage I supplemental factor: a JAK1/2 inhibitor, with Ruxolitinib, used in preferred embodiments; (iii) Stage II factors: an inhibitor of histone demethylation, with Tranylcypromine being used in the preferred embodiments, DNA methyltransferase inhibitor, with 5-Azacytidine being used in preferred embodiments (although Decitabine and RG108 can also be used as the epigenetic modulator), and a c-Jun Kinase inhibitor, with JNKIN8 being used in preferred embodiments (although Sp600125; JNK-in-5; or JNK-in-7; JNK-in-12 can also be used as the c-Jun Kinase inhibitor).

The Stage II condition is added to cells treated with the stage I condition, to upregulate one or more pluripotency-related transcriptional factors and can optionally be supplemented with stage II supplemental factors such as a G9a inhibitor, with UNC0224 being used in the preferred embodiments (although Unc0638 and Unc0321 can be used as the G9a inhibitor), a JAK1/2 inhibitor with Ruxolitinib being used in the preferred embodiments (although Tofacitinib; and AZD1480 can be used as the JAK1/2 inhibitor), a p38 MAPK inhibitor with BIRB796 being used in the preferred embodiments, a BMP receptors/AMPK inhibitor with Dorsomorphin being used in the preferred embodiments, and a CBP/p300 bromodomain inhibitor SGC-CBP30 being used in the preferred embodiments.

Small molecules for Stage III of the reprogramming process include a combination of small molecules with select biological activities (herein, Stage III condition) selected from: (i) Stage I factors: a glycogen kinase inhibitor, with CHIR99021, being used in preferred embodiments, a TGFβ inhibitor, with 616452 being used in preferred embodiments; (ii) Stage I supplemental factors: an SAH hydrolase inhibitor with DZNep being used in the preferred embodiments, a DOT1L methyltransferase inhibitor, with EPZ004777 acid being used in the preferred embodiments; (iii) a Stage II factor: an inhibitor of histone demethylation with Tranylcypromine being used in the preferred embodiment; and (iv) Stage III factors: a histone acetylator/deacetylase inhibitor, with Valproic acid being used in the preferred embodiments, a selective inhibitor of Rho-associated, coiled-coil containing protein kinase (ROCK), with Y27632 being used in the preferred embodiments and a MAPK inhibitor, with PD0325901 being used in preferred embodiments (although AZD8330, TAK-733 and Tramitinib can also be used in place of PD0325901), and a SETD8 inhibitor, with Unc0379 being used in the preferred embodiments.

The Stage III condition is added to cells treated with the stage II condition, to establish an initial pluripotency network, measured by the expression of OCT4.

Small molecules for Stage IV of the reprogramming process include a combination of small molecules with select biological activities (herein, Stage IV condition) are selected from: (i) the Stage I factors: a glycogen kinase inhibitor, with CHIR99021, being used in preferred embodiments and a selective inhibitor of Rho-associated, coiled-coil containing protein kinase (ROCK), with Y27632 being used in the preferred embodiments; (ii) the Stage I supplemental factors: an SAH hydrolase inhibitor with DZNep being used in the preferred embodiments, a DOT1L methyltransferase inhibitor, with EPZ004777 acid being used in the preferred embodiments; (iii) a Stage II factor: an inhibitor of histone demethylation with Tranylcypromine being used in the preferred embodiment; (iv) the Stage III factors: a MAPK inhibitor, with PD0325901 being used in preferred embodiments, a histone acetylator/deacetylase inhibitor, with Valproic acid being used in the preferred embodiments, and (v) stage IV factors: a B-Raf inhibitor, with SB590885 being used in preferred embodiments and a Wnt inhibitor, with IWP2 being used in the preferred embodiments.

In some preferred embodiments, the small molecules of stage I are selected from the following combination: CHIR99021+616452+TTNPB, CHIR99021+616452+CH55, CHIR99021+616452+AM580, CHIR99021+A8301+TTNPB, CHIR99021+A8301+CH55, CHIR99021+A8301+AM580, CHIR99021+SB431542+TTNPB, CHIR99021+SB431542+CH55, CHIR99021+SB431542+AM580, CHIR99021+LY2109761+TTNPB, CHIR99021+LY2109761+CH55, CHIR99021+LY2109761+AM580, TD114-2+616452+TTNPB, TD114-2+616452+CH55, TD114-2+616452+AM580, TD114-2+A8301+TTNPB, TD114-2+A8301+CH55, TD114-2+A8301+AM580, TD114-2+SB431542+TTNPB, TD114-2+SB431542+CH55, TD114-2+SB431542+AM580, TD114-2+LY2109761+TTNPB, TD114-2+LY2109761+CH55, TD114-2+LY2109761+AM580, CHIR98014+616452+TTNPB, CHIR98014+616452+CH55, CHIR98014+616452+AM580, CHIR98014+A8301+TTNPB, CHIR98014+A8301+CH55, CHIR98014+A8301+AM580, CHIR98014+SB431542+TTNPB, CHIR98014+SB431542+CH55, CHIR98014+SB431542+AM580, CHIR98014+LY2109761+TTNPB, CHIR98014+LY2109761+CH55, CHIR98014+LY2109761+AM580, GSK3bi XV+616452+TTNPB, GSK3bi XV+616452+CH55, GSK3bi XV+616452+AM580, GSK3bi XV+A8301+TTNPB, GSK3bi XV+A8301+CH55, GSK3bi XV+A8301+AM580, GSK3bi XV+SB431542+TTNPB, GSK3bi XV+SB431542+CH55, GSK3bi XV+SB431542+AM580, GSK3bi XV+LY2109761+TTNPB, GSK3bi XV+LY2109761+CH55, GSK3bi XV+LY2109761+AM580.

In some preferred embodiments, the small molecules of stage II are selected from the following combination: CHIR99021+616452+TTNPB+SAG+JNK-in-8, CHIR99021+616452+TTNPB+SAG+JNK-in-7, CHIR99021+616452+TTNPB+SAG+JNK-in-12, CHIR99021+616452+TTNPB+Purmorphamine+JNK-in-8, CHIR99021+616452+TTNPB+Hh-ag-1.5+JNK-in-8, CHIR99021+616452+CH55+SAG+JNK-in-8, CHIR99021+616452+AM580+SAG+JNK-in-8, CHIR99021+A8301+TTNPB+SAG+JNK-in-8, CHIR99021+SB431542+TTNPB+SAG+JNK-in-8, CHIR99021+LY2109761+TTNPB+SAG+JNK-in-8, TD114-2+616452+TTNPB+SAG+JNK-in-8, CHIR98014+616452+TTNPB+SAG+JNK-in-8, GSK3bi XV+616452+TTNPB+SAG+JNK-in-8.

In some preferred embodiments, the small molecules of stage III are selected from the following combination: VPA+Dznep+PD0325901+616452, VPA+Dznep+AZD8330+616452, VPA+Dznep+TAK733+616452, VPA+Dznep+Tramitinib+616452, VPA+Adox+PD0325901+616452, VPA+Adox+AZD8330+616452, VPA+Adox+TAK733+616452, VPA+Adox+Tramitinib+616452, VPA+Nepa+PD0325901+616452, VPA+Nepa+AZD8330+616452, VPA+Nepa+TAK733+616452, VPA+Nepa+Tramitinib+616452, MS275+Dznep+PD0325901+616452, MS275+Dznep+AZD8330+616452, MS275+Dznep+TAK733+616452, MS275+Dznep+Tramitinib+616452, MS275+Adox+PD0325901+616452, MS275+Adox+AZD8330+616452, MS275+Adox+TAK733+616452, MS275+Adox+Tramitinib+616452, MS275+Nepa+PD0325901+616452, MS275+Nepa+AZD8330+616452, MS275+Nepa+TAK733+616452, MS275+Nepa+Tramitinib+616452, LMK235+Dznep+PD0325901+616452, LMK235+Dznep+AZD8330+616452, LMK235+Dznep+TAK733+616452, LMK235+Dznep+Tramitinib+616452, LMK235+Adox+PD0325901+616452, LMK235+Adox+AZD8330+616452, LMK235+Adox+TAK733+616452, LMK235+Adox+Tramitinib+616452, LMK235+Nepa+PD0325901+616452, LMK235+Nepa+AZD8330+616452, LMK235+Nepa+TAK733+616452, LMK235+Nepa+Tramitinib+616452, Butyrate+Dznep+PD0325901+616452, Butyrate+Dznep+AZD8330+616452, Butyrate+Dznep+TAK733+616452, Butyrate+Dznep+Tramitinib+616452, Butyrate+Adox+PD0325901+616452, Butyrate+Adox+AZD8330+616452, Butyrate+Adox+TAK733+616452, Butyrate+Adox+Tramitinib+616452, Butyrate+Nepa+PD0325901+616452, Butyrate+Nepa+AZD8330+616452, Butyrate+Nepa+TAK733+616452, Butyrate+Nepa+Tramitinib+616452.

In some preferred embodiments, the small molecules of stage IV are selected from the following combination: PD0325901+SB590885, PD0325901+Sorafinib, PD0325901+GDC0879, AZD8330+SB590885, AZD8330+Sorafinib, AZD8330+GDC0879, TAK733+SB590885, TAK733+Sorafinib, TAK733+GDC0879, Tramitinib+SB590885, Tramitinib+Sorafinib, Tramitinib+GDC0879.

### GSK (Glycogen Synthesis Kinase) Inhibitors

The disclosed reprogramming protocol involves inhibition of GSK in the cell being reprogrammed. The preferred GSK inhibitor is the aminopyrimidine, CHIR99021 having the chemical name [6-[[2-[[4-(2,4-Dichlorophenyl)-5-(5-methyl-1H-imidazol-2-yl)-2-pyrimidinyl]amino]ethyl]amino]-3-pyridinecarbonitrile]. Other GSK inhibitors can also be used in the methods disclosed herein, and they include, but are not limited to BIO-acetoxime; GSK 3I inhibitor XV; SB-216763 [3-(2,4-Dichlorophenyl)-4-(1-methyl-1H-indol-3-yl)-1H-pyrrole-2,5-dione]; CHIR 99021 trihydrochloride, which is the hydrochloride salt of CHIR99021; GSK-3 Inhibitor IX [((2Z, 3E)-6′-bromo-3-(hydroxyimino)-[2,3′-biindolinylidene]-2′-one]; GSK 3 IX [6-Bromoindirubin-3′-oxime]; GSK-3β Inhibitor XII [3-[[6-(3-Aminophenyl)-7H-pyrrolo[2,3-d]pyrimidin-4-yl]oxy]phenol]; GSK-3 Inhibitor XVI [6-(2-(4-(2,4-dichlorophenyl)-5-(4-methyl-1H-imidazol-2-yl)-pyrimidin-2-ylamino)ethyl-amino)-nicotinonitrile]; SB-415286 [3-[(3-chloro-4-hydroxyphenyl)amino]-4-(2-nitrophenyl)-1 H-pyrrole-2,5-dione]; Bio [(2′Z,3′E)-6-bromoindirubin-3′-oxime]; TD114-2 [6,7,9,10,12,13,15,16,18,19-Decahydro-5, 29:20, 25-dimetheno-26H-dibenzo[n, t] pyrrolo[3, 4-q][1,4,7,10,13,22]tetraoxadiazacyclote tracosine-26,28(27H)-dione]; and CHIR 98014 [N6-[2-[[4-(2,4-Dichlorophenyl)-5-(1H-imidazol-1-yl)-2-pyrimidinyl]amino]ethyl]-3-nitro-2,6-pyridinediamine] used at a concentration equivalent to 3-12 μM CHIR99021.

### TGFβ Receptor Inhibitor

The disclosed reprogramming protocol involves inhibition of TGFβ in the cell being reprogrammed. The TGFβ inhibitor is preferably inhibits the TGFβ type 1 receptor activing receptor-like kinase (ALK) 5 in some embodiments, and can additionally inhibit ALK 4 and the nodal type receptor 1 receptor ALK7 in other embodiments.

The preferred TGFβ receptor inhibitor is 616452 [2-(3-(6-Methylpyridin-2-yl)-1H-pyrazol-4-yl)-1,5-naphthyridine]. Other TGFβ inhibitors are known in the art and are commercially available. Examples include A 83-01 [3-(6-Methyl-2-pyridinyl)-N-phenyl-4-(4-quinolinyl)-1 H-pyrazole-1-carbothioamide]; SB 505124 [2-[4-(1,3-Benzodioxol-5-yl)-2-(1,1-dimethylethyl)-1H-imidazol-5-yl]-6-methyl-pyridine]; GW 788388 [4-[4-[3-(2-Pyridinyl)-1H-pyrazol-4-yl]-2-pyridinyl]-N-(tetrahydro-2H-pyran-4-yl)-benzamide]; and SB 525334 [6-[2-(1,1-Dimethylethyl)-5-(6-methyl-2-pyridinyl)-1H-imidazol-4-yl]quinoxaline], and dorsomorphine.

### Histone Acetylator/Deacetylase Inhibitors

The disclosed reprogramming protocol involves inhibition of histone deacetylation, in the cell being reprogrammed. The preferred histone deacetylase inhibitor is valproic acid. However, other histone deacetylase inhibitors are commercially available and can be used. Non-limiting examples include apicidin [cyclo(N-O-methyl-L-tryptophanyl-L-isoleucinyl-D-pipecolinyl-L-2-amino-8-oxodecanoyl)]; LMK235 [N-[[6-(Hydroxyamino)-6-oxohexyl]oxy]-3,5-dimethylbenzamide]; MS275 [(Pyridin-3-yl)methyl 4-(2-aminophenylcarbamoyl)benzylcarbamate]; CI 994 [N-acetyldinaline4-(Acetylamino)-N-(2-aminophenyl)benzamide]; Depsipeptide; KD 5170 [S-[2-[6-[[[4-[3-(Dimethylamino)propoxy]phenyl]sulfonyl]amino]-3-pyridinyl]-2-oxoethyl]ethanethioc acid ester]; sodium,4-pehynl butyrate; sodium butyrate [Butanoic acid sodium salt]; UF 010 [4-Bromo-N-butylbenzohydrazide) and HDACi IV etc.

### Inhibitors of Histone Demethylation

The disclosed reprogramming protocol involves inhibition of histone demethylation in the cell being reprogrammed. A preferred inhibitor of histone demethylation is tranylcypromine. Tranylcypromine is a nonselective and irreversible monoamine oxidase inhibitor (MAOI). Another useful MAOI which are also inhibitors of histone demethylation include RN-1 [2-((1R, 2S)-2-(4-(benzyloxy)phenyl)cyclopropylamino)-1-(4-methylpiperazin-1-yl)ethanone dihydrochloride]; GSK2879 [4-{[4-({[(1R,2S)-2-phenylcyclopropyl]amino}methyl)piperidin-1-yl]methyl}benzoic acid]; S2101 [(1R,2S)-2-[2-(benzyloxy)-3,5-difluorophenyl]cyclopropan-1-amine hydrochloride]; LSD1-C76 [(1R,2S)-N-(1-(2,3-dihydrobenzo[b][1,4]dioxin-6-yl)ethyl)-2-phenylcyclopropanamine], etc.

### Retinoic Acid Receptor (RAR) Agonists

The disclosed reprogramming protocol involves activation of the retinoic acid receptor in the cell being reprogrammed. A preferred RAR agonist is TTNPB [4-[(E)-2-(5,6,7,8-Tetrahydro-5,5,8,8-tetramethyl-2-naphthalenyl)-1-propenyl]benzoic acid]. Others which may be used include Ch 55 [4-[(1E)-3-[3,5-bis(1,1-Dimethylethyl)phenyl]-3-oxo-1-propenyl]benzoic acid], a highly potent synthetic retinoid that has high affinity for RAR-α and RAR-β receptors and low affinity for cellular retinoic acid binding protein (CRABP)]; AM580 ([4-[(5,6,7,8-Tetrahydro-5,5,8,8-tetramethyl-2-naphthalenyl)carboxamido]benzoic acid]; an analog of retinoic acid that acts as a selective RARα agonist);

### SAH Hydrolase Inhibitors

The disclosed reprogramming protocol involves inhibition of SAH in the cell being reprogrammed. The preferred SAH hydrolase inhibitor is 3-deazaneplanocin A (DZNep) [(1S,2R,5R)-5-(4-Amino-1H-imidazo[4,5-c]pyridin-1-yl)-3-(hydroxymethyl)-3-cyclopentene-1,2-diol]. Other useful SAH hydrolase inhibitors that can be included in the CIP combination compositions disclosed herein include, but are not limited to, (−) Neplanocin A (NepA) [5R-(6-amino-9H-purin-9-yl)-3-(hydroxymethyl)-3-cyclopentene-1S,2R-diol]; Adenozine periodate oxidized (Adox) [(2S)-2-[(1R)-1-(6-aminopurin-9-yl)-2-oxoethoxy]-3-hydroxypropanal] and 3-deazaadenosine (DZA) [1-β-D-ribofuranosyl-1H-imidazo[4,5-c]pyridin-4-amine] and combinations thereof.

### DOT1L Methyltransferase Inhibitors

The disclosed reprogramming protocol involves inhibition DOT1L methyltransferase in the cell being reprogrammed. Preferred DOT1L methyltransferase inhibitors include SGC 0946 [1-[3-[[[(2R,3S,4R,5R)-5-(4-Amino-5-bromo-7H-pyrrolo[2,3-d]pyrimidin-7-yl)-3,4-dihydroxytetrahydrofuran-2-yl]methyl](isopropyl)amino]propyl]-3-[4-(2,2-dimethylethyl)phenyl]urea]; EPZ004777 [7-[5-Deoxy-5-[[3-[[[[4-(1,1-dimethylethyl)phenyl]amino]carbonyl]amino]propyl](1-methylethyl)amino]-β-D-ribofuranosyl]-7H-pyrrolo[2,3-d]pyrimidin-4-amine]; EPZ5676 [(2R,3R,4S,5R)-2-(6-amino-9H-purin-9-yl)-5-((((1r,3S)-3-(2-(5-(tert-butyl)-1H-benzo[d]imidazol-2-yl)ethyl)cyclobutyl)(isopropyl)amino)methyl)tetrahydrofuran-3,4-diol]

### Receptor Tyrosine Kinase Inhibitors

A preferred receptor tyrosine kinase inhibitor is ABT 869 (Linifanib) [N-[4-(3-amino-1H-indazol-4-yl)phenyl]-N′-(2-fluoro-5-methylphenyl)-urea], an ATP-competitive receptor tyrosine kinase inhibitor which is a potent inhibitor of members of the vascular endothelial growth factor (VEGF) and platelet derived growth factor (PDGF) receptor families. Other tyrosine kinase inhibitors such as AG1296 [6,7-Dimethoxy-3-phenylquinoxaline] and Valatanib are able to replace ABT 869 and can be used in place of ABT 869.

### B-Rafinhibitors

The disclosed reprogramming protocol involves inhibition B-Raf in the cell being reprogrammed. A preferred B-Raf inhibitor is SB590885 [5-[2-[4-[2-(Dimethylamino)ethoxy]phenyl]-5-(4-pyridinyl)-1H-imidazol-4-yl]-2,3-dihydro-1H-inden-1-one oxime]. SB590885 is a potent B-Raf inhibitor with Kᵢ of 0.16 nM in a cell-free assay, 11-fold greater selectivity for B-Raf over c-Raf, no inhibition to other human kinases. Other potent B-Raf inhibitor are known and can be used for the same activity as SB590885. Examples include Vemurafenib, RAF265 (CHIR-265) (Selleckhchem catalog No. S2161)) and PLX4720 (Selleckhchem catalog No. S11525).

### Wnt Inhibitors

The disclosed reprogramming protocol involves inhibition Wnt in the cell being reprogrammed. A preferred Wnt inhibitor is IWP-2 [N-(6-methyl-2-benzothiazolyl)-2-[(3,4,6,7-tetrahydro-4-oxo-3-phenylthieno[3,2-d]pyrimidin-2-yl)thio]-acetamide]. However, Wnt inhibitors such as WNT-C59 [4-(2-Methyl-4-pyridinyl)-N-[4-(3-pyridinyl)phenyl]benzeneacetamide], XAV-939 [3,5,7,8-Tetrahydro-2-[4-(trifluoromethyl)phenyl]-4H-thiopyrano[4,3-d]pyrimidin-4-one] and IWR-1 (Selleckchem catalog No. S7086) are able to replace IWP-2 and can therefore be used in place of IWP-2.

### Rho-Associated, Coiled-Coil Containing Protein Kinase (ROCK) Inhibitors

The disclosed reprogramming protocol involves inhibition ROCK in the cell being reprogrammed. A preferred ROCK inhibitor is Y27632 ([(+)-(R)-trans-4-(1-aminoethyl)-N-(4-pyridyl)cyclohexanecarboxamide+++dihydrochloride)]) or Tzv (thiazovivin).

### CBP/p300 Bromodomain Inhibitors

The disclosed reprogramming protocol involves inhibition CBP/p300 bromodomain in the cell being reprogrammed. A preferred CBP/p300 bromodomain inhibitor is SGC-CBP30 [2-[2-(3-chloro-4-methoxyphenyl)ethyl]-5-(3,5-dimethyl-4-isoxazolyl)-1-[(2S)-2-(4-morpholinyl)propyl]-1H-benzimidazole], I-CBP112, GNE272, or GNE409.

### Menin-MLL Interaction Inhibitors

The disclosed reprogramming protocol involves inhibition Menin-MLL interaction in the cell being reprogrammed. A preferred Menin-MLL interaction is VTP50469, MI3454, or WDR5-IN-4.

### B. Cells to be Induced

The hCiPSCs are obtained from human somatic cells. A somatic cell as would be understood by one of ordinary skill in the art is any cell other than a gamete (sperm or egg), germ cell (cells that go on to become gametes), gametocyte or undifferentiated stem cell.

The somatic cells can be obtained from tissue such as bone marrow, fetal tissue (e.g., fetal liver tissue), peripheral blood, umbilical cord blood, pancreas, skin or any organ or tissue. In a preferred embodiment, the hCiPSCs are obtained from fibroblasts, adipose-derived cells, neural cells or cells from the intestinal epithelium. In a more preferred embodiment, hCiPSCs are obtained from neonatal (for example foreskin) or adult fibroblasts. However, hCiPSCs can be obtained from other cell types including but not limited to: somatic cells of hematological origin, skin derived cells, adipose cells, epithelial cells, endothelial cells, cells of mesenchymal origin, parenchymal cells (for example, hepatocytes), neurological cells, and connective tissue cells.

In a preferred embodiment, the hCiPSCs are obtained from fibroblasts and adipose-derived somatic cells (for example, adipocytes). In a more preferred embodiment, hCiPSCs are obtained from fibroblast, which can be neonatal (for example foreskin fibroblasts) or adult fibroblast.

Cells may be isolated by disaggregating an appropriate organ or tissue which is to serve as the cell source using techniques known to those skilled in the art. For example, the tissue or organ can be disaggregated mechanically and/or treated with digestive enzymes and/or chelating agents that weaken the connections between neighboring cells, so that the tissue can be dispersed to form a suspension of individual cells without appreciable cell breakage. Enzymatic dissociation can be accomplished by mincing the tissue and treating the minced tissue with one or more enzymes such as trypsin, chymotrypsin, collagenase, elastase, and/or hyaluronidase, DNase, pronase, dispase etc. Mechanical disruption can also be accomplished by a number of methods including, but not limited to, the use of grinders, blenders, sieves, homogenizers, pressure cells, or insonators.

C. hCiPSCs

hCiPSCs obtained according to the disclosed method, are also provided. The cells are characterized in that they can expand for more than 20 passages, for example, for up to 25, 30, 35, 40, 41, 42, passages, proliferated with a doubling time similar to that of hESCs. hCiPSCs are also characterized in that they express at least one surface marker TRA-1-60, TRA-1-81, and SSEA-4, along with the core pluripotency transcriptional factors OCT4, SOX2 (SRY-Box Transcription Factor 2), NANOG (, DNMT3B (DNA methyltransferase 3 beta), DPPA4 (developmental pluripotency-associated 4), UTF1 (undifferentiated embryonic cell transcription factor 1), ZFP42 (Zinc finger protein 42), PRDM14 (PR-domain containing protein 14) and ZIC3 (Zic Family Member 3)) and NANOG (Nanog Homeobox). These markers were not expressed in the traditional pluripotent stem cells (hESCs and hiPSCs). Functionally, when injected hCiPSCs into immunodeficient mice and the resultant teratomas contain tissues of all 3 germ layers (endo-, ecto-, and mesoderm); hCiPSCs form embryoid bodies in vitro and expressed marker genes of the three germ layers and can be can be subjected to directed differentiation into another committed cell type, for example, hepatocytes, or directed differentiation into progenitor cells such as hematopoietic progenitor cells. Most importantly, the hCiPSCs are preferably, not genetically engineered, i.e., not obtained by a process that includes altering human somatic cells by introducing or removing genetic elements from the cells, for example engineering somatic cells to express one or more markers of pluripotency such as Oct3/4 (octamer-binding transcription factor 3/4), KLF4, nanog and/or cMyc and accordingly, hCiPSCs obtained following the disclosed methods preferably do not contain exogenously introduced Oct3/4, KLF4, NANOG and/or cMyc.

### III. Methods of Making

The disclosed method of reprogramming human somatic cells into pluripotent cells is a four stage cell culture process. The human somatic cell to be reprogrammed is harvested from the desired tissue, using methods that are well known in the art and exemplified herein in the Examples below, with adult dermis tissues, Adult human adipose derived mesenchymal stromal cells and Human embryonic fibroblasts. The harvested cells are maintained in culture and passaged, until reprogramming

At stage I, the somatic cells are seeded in cell culture medium, for example, DMEM, KnockOut™ DMEM, DMEM/F12, Advanced DMEM/F12 and exposed to the Stage I condition, in an appropriate cell culture medium for example DMEM, i.e., cell culture medium is supplemented with Stage I factors in effective amounts, preferably the next day, then cultured under conditions of hypoxia with 5% O₂ in the Stage I condition in some embodiments (for example, when the somatic cell is a somatic cell obtained from adult tissue, for example, with hADSCs and hASFs) or 37° C. with 21% O₂ and 5% CO₂ in other embodiments (for example, when the somatic cell is a somatic cell obtained from non-adult tissue, such as HEFs for example) for an effective amount of time to convert the somatic cells into a monolayer epithelial-like cells. This initial induction stage can be commenced from 4 hours to 48 hours after initial seeding of the cells. The cells are seeded at an appropriate density, for example, ADSCs and hASFs can be seeded at a density of 1×10⁴ cells per well of a 12-well plate in 15% FBS DMEM medium. The supplemented medium can be changed every 3-4 days. The length of time in culture effective for conversion of the somatic cells into a monolayer epithelial-like cells will vary depending on the somatic cell type. For example, single layer epithelial-like cells induced from hADSCs can emerge at day 4-6 and approach 80%-100% confluence at day 8-12. For ASFs, epithelial-like cells approach 80%-100% confluence at day 12-20. Conversion into epithelial-like cells can be measured by upregulation of and epithelial cell-related genes, such as KRT8 (for example, up to 60 fold upregulation), KRT18 (for example, up to 22 fold upregulation), and KRT19 for example, up to 2.5 fold upregulation), when compared to the corresponding somatic cells cultured in cell culture medium, without supplementation with the Stage I factors/cultured in the Stage I condition. Additionally, cells at the end of Stage I show increased expression of LIN28A for example, up to 21 fold upregulation when compared to the somatic cells from which they were cultured.

A preferred combination of factors for the Stage I condition are selected from: CHIR99021 (3-12 μM, preferably 10-12 μM, for example, 8, 9, 10, 11, or 12 μM), 616452 (2-50 μM, preferably 5-20 μM, for example, 5, 8, 10, 11, 12, 13, 14, 15 or 20 μM), TTNPB (0.5-10 μM, preferably 1-5 μM, for example, 0.9, 1, 1.5, 2, 2.5, or 3 μM), Y27632 or TZV (1-10 μM, preferably 1-5 μM, for example, 0.9, 1, 1.5, 2, 2.5, or 3 μM), ABT869 (0.5-5 μM, preferably 1-2 μM, for example, 0.6, 0.7, 0.8, 0.9, 1, 1.2 or 1.5 μM) and SAG (0.2-2 μM, preferably 0.5-1 μM, for example, 0.3, 0.4, 0.5, 0.6, 0.7, 0.8 μM). Somatic cells, for example fibroblasts are converted into a monolayer epithelial-like cells, by culturing the cells in cell culture medium supplemented with Stage I factors (Stage I Condition) for an effective amount of time to convert the cells into a monolayer epithelial-like cells. In some embodiments, the Stage I condition could include supplementation with small molecules selected from the group consisting of Dot1L inhibitor (EPZ004777 or EPZ5676 (0.2-10μM, preferably 1-5 μM, for example, 0.9, 1, 1.5, 2, 2.5, or 3 μM), Ruxolitinib (0.1-5 μM, preferably 0.5-1 μM, for example, 0.6, 0.7, 0.8, 0.9, 1, 1.2 or 1.5 μM)) and DZNep (0.005-0.1 μM, preferably, 0.01 to 0.05 for example, 0.01, 0.02, 0.03, 0.04, 0.05 μM).

At stage II, the cell culture medium for the cells treated with the Stage I condition is changed to the Stage II condition for an effective amount of time (for example, 8-20 days in culture) to upregulate one or more pluripotency-related transcriptional factor expression in the cultured cells, measured for example as activation of the pluripotency-related transcriptional factor SALL4 (for example, up to 24 fold upregulation), co-expressed with LIN28A (for example, up to 38 fold upregulation), when compared to the corresponding somatic cells cultured in cell culture medium, without supplementation with the Stage II factors/cultured in the Stage II condition. A preferred combination of factors for the Stage II condition include (i) stage II factors 5-Azacytidine (2-10 μM, preferably 5-10 μM, for example, 5, 6, 7, 8, 9, 10 μM), Tranylcypromine (2-50 μM, preferably 2-10 μM, for example, 2, 2.5, 3, 5, 8 or 10), and JNKIN8 (0.2-2 μM,, preferably 0.5-1 μM, for example, 0.6, 0.7, 0.8, 0.9, 1, 1.2 or 1.5 μM); and (ii) stage I factors: CHIR99021, 616452, TTNPB, ABT-869 and SAG used at the same concentrations as disclosed above for the Stage I condition. The Stage II condition can include additional supplementation of the culture medium with small molecules selected from the group consisting of the stage I supplemental factor Ruxolitinib, used at the same concentrations as disclosed above for the Stage I condition, and stage II supplemental factors: UNC0224 (0.1-5 μM, preferably 0.5-2 μM, for example, 0.6, 0.7, 0.8, 0.9, 1, 1.2 or 1.5 μM), BIRB796 (0.2-5 μM, preferably 2-5 μM, for example, 2, 2.5, 3, 3.5, 4 or 5 μM), and Dorsormorphin (0.2-2 μM, preferably 0.5-1 μM, for example, 0.2, 0.5, 0.6, 0.8 or 1). The length of time in culture effective for culture under the Stage II condition will vary slightly, depending on the cell type. Following culture of hADSCs and hASFs in the Stage II condition, multi-layered colonies appear after about 8-12 days treatment and these cell colonies continue to grow bigger. After a total of about 16-20 days' Stage II condition, the cell culture medium can be changed to the Stage III condition.

At stage III, the cell culture medium for the cells treated with the Stage II condition is changed to the Stage III condition and cultured in the Stage III condition for an effective amount of time to establish an initial pluripotency network, measured by the expression of OCT4. Cells treated with the stage II condition, by culturing the cells in cell culture medium supplemented with Stage III, factors for an effective amount of time to establish an initial pluripotency network, measured by the expression of OCT4 (Stage III condition). A preferred combination of factors for the Stage III condition include: (i) Stage III factors: PD0325901 (0.02-5 μM, preferably 0.5-1 μM, for example, 0.5, 0.6, 0.7, 0.8, 0.9, 1, 1.2 or 1.5 μM), and VPA (200-1500 μM, preferably 200-500 μM, for example, 200, 300, 400 or 500 μM); (ii) the Stage I factor CHIR99021 (1-10μM, preferably 1-3μM, for example, 1, 1.5, 2, 2.5 or 3μM); 616452 used at the same concentrations disclosed for the Stage I condition and Y-27632 (2-50 μM, preferably 2-10 μM, for example, 2, 3, 4, 5, 6, 7, 8, 9 or 10 μM); (iii) a Stage I supplemental factor, DZNep (0.05-0.5 μM, preferably 0.1-0.3 μM, for example, 0.1, 0.15, 0.2, 0.25, or 0.3 μM), and EPZ004777 (0.25-20 μM, preferably 1-10 μM, for example, 1,2,3,4,5,8,or 10 μM); (iv) a Stage II factor Tranylcypromine (5-50 μM, preferably 5-20 μM, for example 5, 8, 10, 15 or 20 μM). The length of time in culture effective for culture under the Stage II condition will vary slightly, depending on the cell type. However, 8-12 days' treatment in the Stage III condition is preferred, in some embodiments, 8-10 days can be used.

At stage IV, the cell culture medium for the cells treated with the Stage III condition is changed to the Stage IV condition and cultured in the Stage IV condition for an effective amount of time to fully establish a pluripotent network, measured by co-expression OCT4, SOX2, and NANOG. A preferred combination of factors for the Stage IV condition selected from: (i) the Stage I factor CHIR99021 (0.2-3 μM, preferably 0.2-1 μM, for example, 0.2, 0.3, 0.5, 0.8 or 1 μM); and Y-27632 (2-20 μM, preferably 2-10 μM, for example, 2, 3, 4, 5, 7, 8, or 10 μM); (ii) the Stage I supplemental factors DZNep (0.02-0.2 μM, preferably 0.02-0.05 μM, for example, 0.02, 0.03, 0.04, 0.05 or 0.1 μM) and EPZ004777 (0.25-20 μM, preferably 1-10 μM, for example, 1, 2, 3, 4, 5, 8 or 10 μM); (iii) the Stage II factor, Tranylcypromine (2-50 μM, preferably 2-10 μM, for example, 2, 2.5, 3, 5, 8 or 10 μM); (iv) the Stage III factors, PD0325901 (0.02-5 μM, preferably 0.1-1μM, for example, 0.1, 0.2, 0.5, 0.7, 0.8 or 1 μM) and VPA (200-1500 μM, preferably 200-500 μM, for example, 200, 300, 400 or 500 μM); and (v) Stage IV factors IWP2 (0.5-4 μM, preferably 1-2 μM, for example, 0.9, 1, 1.5, 2, 2.5, or 3 μM), and SB590885 (0.1-5 μM, preferably 0.2-1 μM, for example, 0.3, 0.4, 0.5, 0.6, 0.7, 0.8 μM). For stage IV induction, VPA, Tranylcypromine, DZnep, and EPZ004777 should preferably be included in the first 4 days of Stage IV condition. Primary hCiPSC colonies would emerge after about 6-8 days' treatment. For HEFs VPA should preferably be included in the first 4 days. Primary hCiPSC colonies would emerge after 6-8 days' treatment.

Although specific factors are disclosed in preferred embodiments, they can be readily interchanged with known small molecules which provide the same biological activity, as disclosed under Compositions. Thus alternative compounds with similar biological activities can be used in amounts that correspond with the biological activity provided by the specific concentrations provided herein for specific compounds. Thus, for example, CHIR99021 is a preferred GSK inhibitor, whose concentration range is provided herein. It can however, be replaced with small molecules with the same biological activity (i.e., ability to inhibit GSK) at least to the levels seen with the concentrations disclosed for CHIR99021. It is within the abilities of one of ordinary skill in the art to determine the equivalent amount of replacement factors, based on the concentrations exemplified herein, for species (specific compound listed) within the genus (disclosed biological activity).

B. Isolation of hCiPSC

Media that can maintain the undifferentiated state and pluripotency of ES cells or induce differentiation are known in this field. Differentiation and proliferation abilities of isolated induced pluripotent stem cells can be easily confirmed by those skilled in the art by using confirmation means widely applied to ES cells.

A substantially purified population of hCiPSCs can be obtained, for example, by extraction (e.g., via density gradient centrifugation and/or flow cytometry) from a culture source. Purity can be measured by any appropriate method. The pluripotent cells can be 99%-100% purified by, for example, flow cytometry (e.g., FACS analysis). Human induced pluripotent stem cells can be isolated by, for example, utilizing molecules (e.g., antibodies, antibody derivatives, ligands or Fc-peptide fusion molecules) that bind to a marker (e.g., a TRA-1-81, a TRA-1-60 or a combination of markers) on the induced pluripotent stem cells and thereby positively selecting cells that bind the molecule (i.e., a positive selection). Other examples of positive selection methods include methods of preferentially promoting the growth of a desired cell type in a mixed population of desired and undesired cell types. Alternatively, by using molecules that bind to markers that are not present on the desired cell type, but that are present on an undesired cell type, the undesired cells containing such markers can be removed from the desired cells (i.e., a negative selection). Other negative selection methods include preferentially killing or inhibiting the growth of an undesired cell type in a mixed population of desired and undesired cell types. Accordingly, by using negative selection, positive selection, or a combination thereof, an enriched population of stem cell can be made.

Procedures for separation may include magnetic separation, using antibody-coated magnetic beads, affinity chromatography, cytotoxic agents joined to a monoclonal antibody, or such agents used in conjunction with a monoclonal antibody, e.g., complement and cytotoxins, and “panning” with antibody attached to a solid matrix (e.g., plate), or other convenient technique. Techniques providing accurate separation include fluorescence activated cell sorters, which can have varying degrees of sophistication, e.g., a plurality of color channels, low angle and obtuse light scattering detecting channels, and impedance channels. Antibodies may be conjugated with markers, such as magnetic beads, which allow for direct separation, biotin, which can be removed with avidin or streptavidin bound to a support, or fluorochromes, which can be used with a fluorescence activated cell sorter, to allow for ease of separation of the particular cell type. Any technique may be employed which is not unduly detrimental to the viability of the induced pluripotent stem cells. In one embodiment, the cells are incubated with an antibody against a marker and the cells that stain positive for the marker are manually selected and subcultured.

Combinations of enrichment methods may be used to improve the time or efficiency of purification or enrichment. For example, after an enrichment step to remove cells having markers that are not indicative of the cell type of interest, the cells may be further separated or enriched by a fluorescence activated cell sorter (FACS) or other methodology having high specificity. Multi-color analyses may be employed with a FACS. The cells may be separated on the basis of the level of staining for a particular antigen or lack thereof. Fluorochromes may be used to label antibodies specific for a particular antigen. Such fluorochromes include phycobiliproteins, e.g., phycoerythrin and allophycocyanins, fluorescein, and Texas red.

C. Culture and Preservation of hCiPSC

The hCiPSCs can be expanded in culture and stored for later retrieval and use. Once a culture of cells or a mixed culture of stem cells is established, the population of cells is mitotically expanded in vitro by passage to fresh medium as cell density dictates under conditions conducive to cell proliferation, with or without tissue formation. Such culturing methods can include, for example, passaging the cells in culture medium lacking particular growth factors that induce differentiation (e.g., IGF, EGF, FGF, VEGF, and/or other growth factor). Cultured cells can be transferred to fresh medium when sufficient cell density is reached. Some stem cell types do not demonstrate typical contact inhibition-apoptosis or they become quiescent when density is maximum. Accordingly, appropriate passaging techniques can be used to reduce contact inhibition and quiescence.

Cells can be cryopreserved for storage according to known methods, such as those described in Doyle et al., (eds.), 1995, Cell & Tissue Culture: Laboratory Procedures, John Wiley & Sons, Chichester. For example, cells may be suspended in a “freeze medium” such as culture medium containing 15-20% fetal bovine serum (FBS) and 10% dimethylsulfoxide (DMSO), with or without 5-10% glycerol, at a density, for example, of about 4-10×10⁶ cells/ml. The cells are dispensed into glass or plastic vials which are then sealed and transferred to a freezing chamber of a programmable or passive freezer. The optimal rate of freezing may be determined empirically. For example, a freezing program that gives a change in temperature of −1° C./min through the heat of fusion may be used. Once vials containing the cells have reached −80° C., they are transferred to a liquid nitrogen storage area. Cryopreserved cells can be stored for a period of years.

### IV. Methods of Use

Identification of a readily available source of stem cells, progenitor cells, dedifferentiated cells or cells with plasticity potentials that can give rise to a desired cell type or morphology is important for therapeutic treatments, tissue engineering and research. The cells obtained by methods of this application including the hCiPSCs, the XEN-like cells, plasticity state cells with regeneration program, and epithelia-like cells are readily available source of stem cells, progenitor cells, dedifferentiated cells or cells with plasticity potentials which express at least one of stem cell related markers such as LIN28A, SALL4, OCT4 or NANOG. In this regard, although the term hCiPSC is used as an example, one skilled in the art would understand the XEN-like cells, plasticity state cells with regeneration program, and epithelia-like cells obtained by methods of this application may also be used similarly as source of stem cells, progenitor cells, dedifferentiated cells or cells with plasticity potentials. The availability of stem cells, progenitor cells, dedifferentiated cells or cells with plasticity and regenerative potentials would be extremely useful in transplantation, tissue engineering, and regulation of angiogenesis, vasculogenesis, and cell replacement or cell therapies as well as the prevention of certain diseases. Such stem cells or progenitor cells can also be used to introduce a gene into a subject as part of a gene therapy regimen. In addition, the cells obtained by a method of this application comprising one or more of Stages I, II, III, IV e.g., epithelia-like cells, plasticity state cells with regeneration program, and/or XEN-like cells may be directly induced to a desired cell type and implanted and delivered to the subject, that is, it is not necessary in all cases to obtain hCiPSCs first to obtain differentiated cells.

### A. Providing Differentiated Somatic Cells (Re-Differentiated Cells)

Once established, a culture of stem cells may be used to produce progeny cells, for example, fibroblasts capable of producing new tissue. The hCiPSCs can be induced to differentiate into cells from any of the three germ layers, for example, skin and hair cells including epithelial cells, keratinocytes, melanocytes, adipocytes, cells forming bone, muscle and connective tissue such as myocytes, chondrocytes, osteocytes, alveolar cells, parenchymal cells such as hepatocytes, renal cells, adrenal cells, and islet cells, blood cells, retinal cells (and other cells involved in sensory perception, such as those that form hair cells in the ear or taste buds on the tongue), and nervous tissue including nerves.

In one embodiment, the hCiPSCs are induced to differentiate into cells of ectodermal origin by exposing the cells to an “ectodermal differentiating” media. In another embodiment the hCiPSCs are induced to differentiate into cells of mesodermal origin by exposing the cells to “mesodermal differentiating media”. In still another embodiment, the hCiPSCs are induced to differentiate into cells of endodermal origin by exposing the cells to “endodermal media”. Components of “endodermal”, “mesodermal” and “ectodermal” media are known to one of skill in the art. Known cell surface markers can be used to verify that the cells are indeed differentiating into cells of the lineage of the corresponding cell culture medium. The most commonly accepted markers to confirm differentiation of the three germ layers are the expression of alpha fetal protein for endodermal cells, alpha smooth muscle actin for mesoderm, and Beta-III tubulin for ectoderm, all of which are normally expressed very early in the development of these tissues.

Differentiation of stem cells to fibroblasts or other cell types, followed by the production of tissue therefrom, can be triggered by specific exogenous growth factors or by changing the culture conditions (e.g., the density) of a stem cell culture. Methods for inducing differentiation of cells into a cell of a desired cell type are known in the art. For example, hCiPSCs can be induced to differentiate by adding a substance (e.g., a growth factor, enzyme, hormone, or other signaling molecule) to the cell's environment. Examples of factors that can be used to induce differentiation include erythropoietin, colony stimulating factors, e.g., GM-CSF, G-CSF, or M-CSF, interleukins, e.g., IL-1,-2,-3,-4,-5,-6,-7,-8, Leukemia Inhibitory Factory (LIF), or Steel Factor (Stl), coculture with tissue committed cells, or other lineage committed cells types to induce the stem cells into becoming committed to a particular lineage.

The differentiated cells can be can be expanded in culture and stored for later retrieval and use.

The XEN-like cells, plasticity state cells, and epithelia-like cells are readily available source for generating to other cell types that can be triggered by specific exogenous growth factor, small molecules, over expression genes or by changing the culture conditions (e.g., the density) of a stem cell culture. The cells induced from the XEN-like cells, plasticity state cells, and epithelia-like cells can be different cell types including but not limited to: somatic cells of hematological origin, skin derived cells, adipose cells, epithelial cells, endothelial cells, cells of mesenchymal origin, parenchymal cells (for example, hepatocytes, β cells), neurological cells, and connective tissue cells.

### B. Cell Therapy

Therapeutic uses of the induced pluripotent stem cells include transplanting the induced pluripotent stem cells, stem cell populations, or progeny thereof into individuals to treat a variety of pathological states including diseases and disorders resulting from cancers, wounds, neoplasms, injury, viral infections, diabetes and the like. Treatment may entail the use of the cells to produce new tissue, and the use of the tissue thus produced, according to any method presently known in the art or to be developed in the future. The cells may be implanted, injected or otherwise administered directly to the site of tissue damage so that they will produce new tissue in vivo. In one embodiment, administration includes the administration of genetically modified hCiPSCs or their progeny.

In a preferred embodiment, the hCiPSCs are obtained from autologous cells i.e., the donor cells are autologous. However, the cells can be obtained from heterologous cells. In one embodiment, the donor cells are obtained from a donor genetically related to the recipient. In another embodiment, donor cells are obtained from a donor genetically un-related to the recipient.

If the hCiPSCs are derived from a heterologous (non-autologous/allogenic) source compared to the recipient subject, concomitant immunosuppression therapy is typically administered, e.g., administration of the immunosuppressive agent cyclosporine or FK506. However, due to the immature state of the human induced pluripotent stem cells such immunosuppressive therapy may not be required. Accordingly, in one embodiment, the human induced pluripotent stem cells can be administered to a recipient in the absence of immunomodulatory (e.g., immunosuppressive) therapy. Alternatively, the cells can be encapsulated in a membrane, which permits exchange of fluids but prevents cell/cell contact. Transplantation of microencapsulated cells is known in the art, e.g., Balladur et al., Surgery, 117:189-94, 1995; and Dixit et al., Cell Transplantation 1:275-79 (1992).

### (i) Diabetes

Diabetes mellitus (DM) is a group of metabolic diseases where the subject has high blood sugar, either because the pancreas does not produce enough insulin, or, because cells do not respond to insulin that is produced. A promising replacement for insulin therapy is provision of islet cells to the patient in need of insulin. Shapiro et al., N Engl J Med., 343(4):230-8 (2000) have demonstrated that transplantation of beta cells/islets provides therapy for patients with diabetes. Although numerous insulin types are commercially available, these formulations are provided as injectables. The human induced pluripotent stem cells provide an alternative source of islet cells to prevent or treat diabetes. For example, induced pluripotent stem cells can be isolated and differentiated to a pancreatic cell type and delivered to a subject. Alternatively, the induced pluripotent stem cells can be delivered to the pancreas of the subject and differentiated to islet cells in vivo. Accordingly, the cells are useful for transplantation in order to prevent or treat the occurrence of diabetes. Methods for reducing inflammation after cytokine exposure without affecting the viability and potency of pancreatic islet cells are disclosed for example in U.S. Pat. No. 8,637,494 to Naziruddin, et al.

### (ii) Neurodegenerative Disorders

Neurodegenerative disorders are characterized by conditions involving the deterioration of neurons as a result of disease, hereditary conditions or injury, such as traumatic or ischemic spinal cord or brain injury. Neurodegenerative conditions include any disease or disorder or symptoms or causes or effects thereof involving the damage or deterioration of neurons. Neurodegenerative conditions can include, but are not limited to, Alexander Disease, Alper's Disease, Alzheimer Disease, Amyotrophic Lateral Sclerosis, Ataxia Telangiectasia, Canavan Disease, Cockayne Syndrome, Corticobasal Degeneration, Creutzfeldt-Jakob Disease, Huntington Disease, Kennedy's Disease, Krabbe Disease, Lewy Body Dementia, Machado-Joseph Disease, Multiple Sclerosis, Parkinson Disease, Pelizaeus-Merzbacher Disease, Niemann-Pick's Disease, Primary Lateral Sclerosis, Refsum's Disease, Sandhoff Disease, Schilder's Disease, Steele-Richardson-Olszewski Disease, Tabes Dorsalis or any other condition associated with damaged neurons. Other neurodegenerative conditions can include or be caused by traumatic spinal cord injury, ischemic spinal cord injury, stroke, traumatic brain injury, and hereditary conditions.

In particular, the disclosed methods include transplanting into a subject in need thereof NSCs, neural progenitors, or neural precursors that have been expanded in vitro such that the cells can ameliorate the neurodegenerative condition. Transplantation of the expanded neural stem cells can be used to improve ambulatory function in a subject suffering from various forms of myelopathy with symptoms of spasticity, rigidity, seizures, paralysis or any other hyperactivity of muscles. Methods for expanding and transplanting neural cells and neural progenitor cells for the treatment of different neurodegenerative conditions is disclosed for example, in U.S. Pat. No. 8,236,299 to Johe, et. al.

(iii) Cancer Therapy

Therapeutic uses of the hCiPSCs and their progeny include transplanting the induced pluripotent stem cells, stem cell populations, or progeny thereof into individuals to treat and/or ameliorate the symptoms associated with cancer. For example, in one embodiment, the hCiPSCs can be administered to cancer patients who have undergone chemotherapy that has killed, reduced, or damaged cells of a subject. In a typical stem cell transplant for cancer, very high doses of chemotherapy are used, often along with radiation therapy, to try to destroy all the cancer cells. This treatment also kills the stem cells in the bone marrow. Soon after treatment, stem cells are given to replace those that were destroyed.

In another embodiment, the hCiPSCs can be transfected or transformed (in addition to the de-differentiation factors) with at least one additional therapeutic factor. For example, once hCiPSCs are isolated, the cells may be transformed with a polynucleotide encoding a therapeutic polypeptide and then implanted or administered to a subject, or may be differentiated to a desired cell type and implanted and delivered to the subject. Under such conditions the polynucleotide is expressed within the subject for delivery of the polypeptide product.

(iii) Tissue Engineering

hCiPSCs and their progeny can be used to make tissue engineered constructions, using methods known in the art. Tissue engineered constructs may be used for a variety of purposes including as prosthetic devices for the repair or replacement of damaged organs or tissues. They may also serve as in vivo delivery systems for proteins or other molecules secreted by the cells of the construct or as drug delivery systems in general. Tissue engineered constructs also find use as in vitro models of tissue function or as models for testing the effects of various treatments or pharmaceuticals. The most commonly used biomaterial scaffolds for transplantation of stem cells are reviewed in the most commonly used biomaterial scaffolds for transplantation of stem cells is reviewed in Willerth, S. M. and Sakiyama-Elbert, S. E., Combining stem cells and biomaterial scaffolds for constructing tissues and cell delivery (Jul. 9, 2008), StemBook, ed. The Stem Cell Research Community, StemBook. Tissue engineering technology frequently involves selection of an appropriate culture substrate to sustain and promote tissue growth. In general, these substrates should be three-dimensional and should be processable to form scaffolds of a desired shape for the tissue of interest.

U.S. Pat. No. 6,962,814 generally discloses method for producing tissue engineered constructs and engineered native tissue. With respect to specific examples, U.S. Pat. No. 7,914,579 to Vacanti, et al., discloses tissue engineered ligaments and tendons. U.S. Pat. No. 5,716,404 discloses methods and compositions for reconstruction or augmentation of breast tissue using dissociated muscle cells implanted in combination with a polymeric matrix. US Patent No. 8,728,495 discloses repair of cartilage using autologous dermal fibroblasts. U.S. Published application No. 20090029322 by Duailibi, et al., discloses the use of stem cells to form dental tissue for use in making tooth substitute. U.S. Published application No. 2006/0019326 discloses cell-seed tissue-engineered polymers for treatment of intracranial aneurysms. U.S. Published application No. 2007/0059293 by Atala discloses the tissue-engineered constructs (and method for making such constructs) that can be used to replace damaged organs for example kidney, heart, liver, spleen, pancreas, bladder, ureter and urethra.

(iv) Cells Produced From hCiPSCs (Progeny)

The hCiPSCs can be induced to differentiate into cells from any of the three germ layers, for example, skin and hair cells including epithelial cells, keratinocytes, melanocytes, adipocytes, cells forming bone, muscle and connective tissue such as myocytes, chondrocytes, osteocytes, alveolar cells, parenchymal cells such as hepatocytes, renal cells, adrenal cells, and islet cells (e.g., alpha cells, delta cells, PP cells, and beta cells), blood cells (e.g., leukocytes, erythrocytes, macrophages, and lymphocytes), retinal cells (and other cells involved in sensory perception, such as those that form hair cells in the ear or taste buds on the tongue), and nervous tissue including nerves.

### (v) Therapeutic Compositions

The hCiPSCs can be formulated for administration, delivery or contacting with a subject, tissue or cell to promote de-differentiation in vivo or in vitrolex vivo. Additional factors, such as growth factors, other factors that induce differentiation or dedifferentiation, secretion products, immunomodulators, anti-inflammatory agents, regression factors, biologically active compounds that promote innervation, vascularization or enhance the lymphatic network, and drugs, can be incorporated.

The induced pluripotent cells can be administered to a patient by way of a composition that includes a population of hCiPSCs or hCiPSC progeny alone or on or in a carrier or support structure. In many embodiments, no carrier will be required. The cells can be administered by injection onto or into the site where the cells are required. In these cases, the cells will typically have been washed to remove cell culture media and will be suspended in a physiological buffer

In other embodiments, the cells are provided with or incorporated onto or into a support structure. Support structures may be meshes, solid supports, scaffolds, tubes, porous structures, and/or a hydrogel. Such solid supports and methods of culturing cells thereon are known in the art.

The invention will be further understood in view of the following non-limiting Examples, which are presented as preferred embodiments.

### C. Use of Small Molecule Compositions

The Small molecule compositions can be used for tissue regeneration, tissue remodeling and repair, rejuvenation or reversing aging, inhibiting or reversing fibrosis, and inducing plasticity in human somatic cells in vitro and in vivo.

For example, small molecules for Stage I and Stage II of the reprogramming process can be formulated for administration, delivery or contacting with a subject, tissue or cell to promote de-differentiation, regeneration, repair and rejuvenation in vivo or in vitro/ex vivo. Additional factors, such as growth factors, other factors that induce dedifferentiation or regeneration, secretion products, immunomodulators, anti-inflammatory agents, regression factors, biologically active compounds that promote innervation, vascularization or enhance the lymphatic network, and drugs, can be incorporated.

In one embodiment, the small molecules can be administered to a patient by way of a composition that includes all or part of the small molecules for Stage I or Stage II of the reprogramming process. The small molecule compositions can be administered systemically or by injection onto or into the site where the cells are lost or tissues are damaged to boost the endogenous repair ability. In some embodiments, no carrier will be required. In other embodiments, the compositions can include a pharmaceutically acceptable carrier. The small molecules can also be formulated for sustained release, for example, using microencapsulation. The small molecule compositions can be administered to a patient in a single dose, in multiple doses, in a continuous or intermittent manner to obtain the desired physiological effect, depending on the recipient's physiological conditions.

In some embodiments small molecules for Stage I and Stage II of the reprogramming process can be formulated for administration, delivery or contacting with a subject, tissue or cell to promote rejuvenation. These small molecules can be formulated to prevent the age-associated histological changes and maintain the cells in a younger state in tissues. The rejuvenating effects can be detected by reversion of the epigenetic clock, or metabolic changes, or transcriptomic changes, such as changes in senescence, stress, or inflammation pathways. The small molecule compositions can be administered to a patient in a single dose, in multiple doses, in a continuous or intermittent manner to obtain the desired physiological effect, depending on the recipient's physiological conditions.

In some embodiments small molecules for Stage I and Stage II of the reprogramming process can be formulated for administration, delivery or contacting with a subject, tissue or cell to inhibit or revise the fibrosis. Fibrosis can be detected by the changes of morphology, epigenome, or metabolic changes, or transcriptomic changes induced by the disease, stress, or inflammations. The small molecule compositions can be administered to a patient in a single dose, in multiple doses, in a continuous or intermittent manner to inhibit or revise the fibrosis, depending on the recipient's physiological conditions.

In some embodiments small molecules for Stage I and Stage II of the reprogramming process can be formulated for administration, delivery or contacting with a subject, tissue or cell to induce cell plasticity in human somatic cells. Cell plasticity can be detected by the changes of epigenome, metabolic changes, or transcriptomic changes. The small molecule compositions can be used in vitro or in vivo in a single dose, in multiple doses, in a continuous or intermittent manner to induce cell plasticity.

### EXAMPLES

### Materials and Methods

Isolation and culture of HEFs

Human embryonic fibroblasts (HEFs) were isolated from embryonic dermis tissues that obtained with informed written consent and approval by the Clinical Research Ethics Committee of China-Japan Friendship Hospital (Ethical approval No. 2009-50). This study was conducted according to the principles of the Declaration of Helsinki. Briefly, the tissues (0.5-1 cm²) were washed twice with PBS (CORNING, 05418005) containing 2% Penicillin-Streptomycin (Gibco, 15140-122), minced by scissors to 1-2 mm² and dissociated with 5-10 ml 2 mg/ml collagenase IV solution (Gibco, 1963347) in a 100-mm dish at 37° C. for 1 hour. Next, 10-20 ml 15% FBS-DMEM medium was added and cells were pipetted up and down several times for dissociation. The suspension was collected to 50-ml tube and shook for 1-2 min to release cells. Then the suspension was centrifuged at 400 g for 5 min and cells were resuspended in 15% FBS-DMEM medium after remove the supernatant. Generally, 1-3×10⁶ cells can be obtained from 0.5-1 cm² dermis and are plated in a 100-mm dish (P0) followed by incubation in 37° C. with 5% CO₂. The next day, fresh 15% FBS-DMEM medium was changed to remove the non-adherent cells. Primary HEFs usually become confluent in 3-4 days and were ready to passage for reprogramming. 0.25% Trypsin-EDTA (Gibco, 25200-056) was used to dissociate primary HEFs. For CiPSCs induction, HEFs were seeded at a density of 1.5×10⁴ cells per well of 12-well plate with 15% FBS-DMEM medium. For culture and expansion, HEFs were seeded at a density of 1.5×10⁶ cells per 100-mm dish. It is recommend to use the primary HEFs for the induction of CiPS cells within 7 passages. The 15% FBS-DMEM medium: high glucose DMEM (Gibco, C11965500BT) supplemented with 15% Fetal Bovine Serum (FBS) (Vistech, VIS93526487), 1% GlutaMAX™ (Gibco, 35050-061), 1% MEM Non-Essential Amino Acids Solution (NEAA) (Gibco, 11140050), 1% Penicillin-Streptomycin and 0.055 mM 2-mercaptoethanol (Gibco, 21985-023).

Isolation and Culture of hADSCs

Adult human adipose derived mesenchymal stromal cells (hADSCs) were isolated from adult adipose tissue that obtained with informed written consent and approval by the Institute of Ethics Committee Review Board in Peking University (IRB 00001052-19070). This study was conducted according to the principles of the Declaration of Helsinki. Briefly, the tissues (2-4 cm²) were washed twice with PBS containing 2% penicillin-streptomycin, minced by scissors to 1-2 mm2 and dissociated with 5-10 ml 2 mg/ml collagenase IV solution in a 100-mm dish at 37° C. for 1 hour. Next, 10-20 ml 15% FBS-DMEM medium was added and cells were pipetted up and down several times for dissociation. The suspension was collected to two 50-ml tubes and diluted to 30-40 ml each with 15% FBS-DMEM medium, followed by shaking for 1-2 min to release cells. Then the suspension was centrifuged at 400 g for 5 min and cells were resuspended in Mesenchymal Stem Cell Growth Medium 2 (Promo Cell, C-28009) after removing the supernatant. Generally, 1-3×10⁶ cells can be obtained from 2-4 cm² adipose tissue and are plated in a 100-mm dish (P0) followed by incubation in 37° C. with 5% CO₂. The next day, fresh Mesenchymal Stem Cell Growth Medium 2 was changed to remove the non-adherent cells. Primary hADSCs usually become confluent in 3-5 days and were ready to passage for reprogramming. The 0.25% Trypsin-EDTA was used to dissociate primary HEFs. For CiPSCs induction, hADSCs were seeded at a density of 1×10⁴ cells per well of a 12-well plate with 15% FBS-DMEM medium. For culture and expansion, hADSCs were seeded at a density of 1.5×10⁶ cells per 100-mm dish with Mesenchymal Stem Cell Growth Medium 2. It is recommend to use the primary hADSCs for the induction of CiPS cells within 4 passages.

Isolation and Culture of hASFs

Human adult skin fibroblasts (hASFs) were isolated from adult dermis tissues that obtained with informed written consent and approval by the Institute of Ethics Committee Review Board in Peking University (IRB 00001052-19070). This study was conducted according to the principles of the Declaration of Helsinki. Briefly, the tissues (0.5-1 cm²) were washed twice with PBS containing 2% penicillin-streptomycin, minced by scissors to 0.5-1 mm² pieces. Then the pieces were placed in the 100-mm cell culture dish and 1 drop of 15% FBS-DMEM medium was put onto each piece of tissue. Next, the pieces were incubated in 37° C. with 5% CO₂ for 4-12 hours (do not allow the pieces go to dry out). Then 3-5 ml of Mesenchymal Stem Cell Growth Medium 2 was mildly added to the dish (do not allow the pieces detached from the dish). Fresh Mesenchymal Stem Cell Growth Medium 2 was replaced every 2-3 days. Within 4-7 days, outgrowths of fibroblasts would generate. The primary hASFs usually become confluent in 10-14 days and were ready to passage for reprogramming. The hASFs were passaged both for reprogramming and expansion in the same way to hADSCs mentioned above.

Generation of hCiPSCs From HEFs
Medium Preparation for hCiPSCs Induction

### Stage I Induction Medium:

KnockOut™ DMEM (Gibco, 10829018) supplemented with 10% Knockout Serum Replacement (KSR) (Gibco, 10828028), 10% FBS, 1% GlutaMAX™, 1% NEAA, 1% Penicillin-Streptomycin, 0.055 mM 2-mercaptoethanol, 50 μg/ml L-Ascorbic acid 2-phosphate (Vc2P) (Sigma, A8960), 5 mM LiCl (Sigma, L4408), 1 mM Nicotinamide (NAM) (Sigma, 72340), 2 mg/mL AlbuMAX™-II (Gibco, 11021045) and the small molecules CHIR999021 (10 μM), 616452 (10 μM), TTNPB (2 μM), SAG (0.5 μM), ABT-869 (1 μM), Rock inhibitor (Y-27632 (2 μM) or Tzv (2 μM))

To enhance the reprogramming efficiency, Dot1L inhibitor (EPZ004777 (0.2 μM) or EPZ5676 (0.2 μM)), Ruxolitinib (1 μM) and DZNep (0.01 μM) was introduced in stage I induction medium.

### Stage II Induction Medium:

KnockOut™ DMEM supplemented with 10% KSR, 10% FBS, 1% GlutaMAX™, 1% NEAA, 1% Penicillin-Streptomycin, 0.055 mM 2-mercaptoethanol, 50 μg/ml Vc2p, 5 mM LiCl, 1 mM NAM, 40 ng/ml bFGF (Origene, TP750002) and the small molecules CHIR99021 (10 μM), 616452 (10 μM), TTNPB (2 μM), SAG (0.5 μM), ABT-869 (1 μM), Y27632 (10 μM), JNKIN8 (1 μM), Tranylcypromine (10 μM), 5-Azacytidine (5 μM).

To enhance the reprogramming efficiency, the small molecules UNC0224 (1 μM), Ruxolitinib (1 μM) (Selleckchem catalog No. S7256) and CBP/p300 bromodomain inhibitor SGC-CBP30 (2μM) can be introduced into the stage II induction medium.

### Stage III Induction Medium:

Knockout™ DMEM supplemented with 1% N2 supplement (Gibco, 17502-048), 2% B27 supplement (Gibco, 17504-044), 1% GlutaMAX™, 1% NEAA, 1% Penicillin-Streptomycin, 0.055 mM 2-mercaptoethanol, 5 mg/mL AlbuMAX™-II, 20 ng/ml Recombinant Human Heregulinβ-1 (HRG) (PEPROTECH, 100-03) and the small molecules CHIR99021 (1 μM), 616452 (10 μM), Y-27632 (10 μM), PD0325901 (1 μM), Tranylcypromine (10 μM), VPA (500 μM), DZNep (0.2 μM), EPZ004777 (5 μM) Stage IV induction medium:

Knockout™ DMEM supplemented with 1% N2 supplement, 2% B27 supplement, 1% GlutaMAX™, 1% NEAA, 1% Penicillin-Streptomycin, 0.055 mM 2-mercaptoethanol, 20 ng/mL HRG and the small molecules CHIR99021 (1 μM), Y-27632 (10 μM), PD0325901 (1 μM), IWP-2 (2 μM), SB590885 (0.5 μM). The VPA (500 μM) was included in the first 4 days.

Induction Process of hCiPSCs From HEFs

Cells were maintained at 37° C. with 21% 02 and 5% CO₂. The induction medium was changed every 3-4 days. HEFs were seeded at a density of 1-1.5×10⁴ cells per well of a 12-well plate in 15%. FBS-DMEM medium. Change the medium into stage I induction medium on the next day. For stage I induction, single layer epithelial-like cells would emerge at day 4-6 and approach 80%-100% confluence at day 8-12, then change the medium into stage II induction medium. For stage II induction, multi-layered colonies appeared after 8-12 days treatment and these cell colonies would continually grow larger. After totally 16-20 days' stage II treatment, change the medium into stage III condition. For stage III induction, 8-12 days treatment of stage III medium is required and then change the medium into stage IV condition. For stage IV induction, VPA (500 μM) was included in the first 4 days. Primary hCiPSC colonies would emerge after 6-8 days' treatment. At the end of stage IV, immunofluorescent staining of co-expression of OCT4 and NANOG was used to confirm the generation of primary hCiPSC colonies. Primary hCiPSC colony number was calculated as the number of the compact OCT4 positive colonies. Reprogramming efficiency was calculated as the number of primary hCiPSC colonies divided by the number of input HEFs.

Generation of hCiPSCs From hADSCs and hASFs
Medium Preparation for hCiPSCs Induction

### Stage I Induction Medium:

KnockOut™ DMEM supplemented with 10% KSR, 10% FBS, 1% GlutaMAX™, 1% NEAA, 0.055 mM 2-mercaptoethanol, 1% Penicillin-Streptomycin, 50 μg/ml Vc2p, 5mM LiCI, 1 mM NAM, 2 mg/mL AlbuMAX™-II and the small molecules CHIR999021 (10 μM), 616452 (10 μM), TTNPB (2 μM), SAG (0.5 μM), ABT-869 (1 μM), Rock inhibitor (Y-27632 (2 μM) or Tzv (2 μM)), Dot1L inhibitor (EPZ004777 (2 μM) or EPZ5676 (2 μM)), DZNep (0.02 μM), Ruxolitinib (1 μM). To enhance the reprogramming efficiency, the Menin-MLL interaction inhibitor VTP50469 was introduced in stage I induction medium.

In several experiments, to testing the reprogramming efficiency after removing each small molecules, the indicated small molecules were removed from the cocktails. The concentration of the small molecules used to regulate the same pathway or targets were indicated as follow: GSK3 inhibitors (CHIR99021: 3-15 μM; TD114-2: 0.5-2 μM; CHIR98014: 1-3 μM; GSK3βi XV: 0.05-0.2 μM); RA pathway agonists (TTNPB: 0.5-10 μM; Ch55: 1-5 μM; AM580: 0.1-1 μM); Rock inhibitors (TZV: 2-10 μM; Y27632: 2-15 μM; Fasudil: 2-10 μM; HA1100: 2-10 μM); TGFβ inhibitors (A8301: 0.1-5 μM; SB431542: 2-50 μM; LY364947: 0.5 μM; LY21: 0.5 μM; 616452: 10 μM).

### Stage II Induction Medium:

KnockOut™ DMEM supplemented with 10% KSR, 10% FBS, 1% GlutaMAX™, 1% NEAA, 0.055 mM 2-mercaptoethanol, 1% Penicillin-Streptomycin, 50 μg/ml Vc2p, 5 mM LiCl, 1 mM NAM, 100 ng/ml bFGF (Origene) and the small molecules CHIR99021 (10-12 μM), 616452 (10 μM), TTNPB (2 μM), SAG (0.5 μM), ABT-869 (1 μM), Y-27632 (10 μM), JNKIN8 (1 μM), Tranylcypromine (2 μM), 5-Azacytidine (5 μM), UNC0224 (1 μM), Ruxolitinib (1 μM), BIRB796 (2 μM), Dorsormorphin (0.5-1 μM). To enhance the reprogramming efficiency, the CBP/p300 bromodomain inhibitor SGC-CBP30 (2 μM) and Menin-MLL interaction inhibitor VTP50469 were introduced in stage II induction medium.

In several experiments, to testing the reprogramming efficiency after regulate each small molecules, the indicated small molecules were removed from the cocktails. The concentration of the small molecules used to targeting the same pathway or targets were indicated as follow: GSK3 inhibitors (CHIR99021: 3-15 μM; TD114-2: 0.5-2 μM; CHIR98014: 1-3 μM; GSK3βi XV: 0.05-0.2 μM); TGFβ inhibitors (A8301: 0.2-5 μM; SB431542: 2-50 μM; 616452: 10 μM); RA pathway agonists (TTNPB: 0.2-10 μM; Ch55: 1-10 μM; AM580: 0.1-1 μM); Rock inhibitors (Y27632: 2-15 μM; Fasudil: 2-10 μM; HA1100: 2-10 μM; TZV: 2-10 μM); Smoothened agonists (SAG: 0.2-2 μM; Purmorphamine: 0.5-2 μM; Hg-Ag1.5: 0.5-1 μM; Human shh: 20-200 μM); histone demethylation inhibitors (Tranylcypromine: 2-50 μM; GSK2879: 0.02-2 μM; LSD-C76: 0.2-5 μM; S2101: 0.5-5 μM; LSD-2D: 0.2-5 μM; RN-1: 0.2-5 μM); DNMT inhibitors (5-azaC: 2-15 μM; Decitabine: 0.5-10 μM; RG108: 0.5-10 μM); JNK inhibitors (JNK-in-8: 0.2-2 μM; JNK-in-5: 0.5-1 μM; JNK-in-7: 0.2-2 μM; JNK-in-12: 0.2-0.5 μM); CBP/p300 bromodomain inhibitor (SGC-CBP30: 0.5-2 μM; I-CBP112: 0.5-5 μM; GNE272: 0.5-5μM; GNE409:0.5-5μM); Menin-MLL interaction inhibitor (VTP50469: 0.5-2μM; MI3454: 0.5-2μM; WDR5-IN-4: 0.5-2μM).

### Stage III Induction Medium:

Knockout™ DMEM supplemented with 1% N2 supplement, 2% B27 supplement, 1% GlutaMAX™, 1% NEAA, 0.055 mM 2-mercaptoethanol, 1% Penicillin-Streptomycin, 5 mg/mL AlbuMAX™-II, 20 ng/mL HRG and the small molecules CHIR99021 (1 μM), 616452 (10 μM), Y-27632 (10 μM), PD0325901 (1 μM), Tranylcypromine (10 μM), VPA (500 μM), Dznep (0.2 μM), EPZ004777 (5 μM),. In several experiments, to testing the reprogramming efficiency after removing each small molecules, the indicated small molecules were removed from the cocktails. The concentration of the small molecules used to targeting the same pathway or targets were indicated as follow: GSK3 inhibitors (CHIR99021: 1-10 μM; TD114-2: 0.05-0.5 μM; TD114-3: 0.2-1 μM; IM12: 0.5-2 μM; CHIR98014: 0.2-1 μM); TGFβ inhibitors (616452: 2-15 μM; A8301: 0.5-5 μM; SB431542: 2-50 μM); Rock inhibitors (Y27632: 2-100 μM; TZV: 2-10 μM; Fasudil: 5-10 μM; Blebbistatin: 2-10 μM); histone demethylation inhibitors (Tranylcypromine: 10-50 μM; RN-1: 1-2 μM; GSK2879: 0.5-1 μM; S2101: 0.5-2μM; LSD-C76: 0.5-2 μM); HDAC inhibitors (VPA: 200-1500 μM; MS275: 0.2-2 μM; LMK235: 0.05-0.5 μM; Butyrate sodium: 200-1000 μM); Dolt1L inhibitors (EPZ004777: 5 μM; EPZ5676: 1-5 μM; SGC0946: 1-5 μM); S-adenosyl-L homocysteine hydrolase inhibitors (DZNep: 0.1-0.5 μM; Adox: 10-70 μM); ERK inhibitors (PD0325901: 0.02-5 μM); SETD8 inhibitors (UNC0379: 0.1-2 μM).

### Stage IV Induction Medium:

Knockout™ DMEM supplemented with 1% N2 supplement, 2% B27 supplement, 1% GlutaMAX™, 1% NEAA, 0.055 mM 2-mercaptoethanol, 1% Penicillin-Streptomycin, 20 ng/mL HRG and the small molecules CHIR99021 (1 μM), Y-27632 (10 μM), PD0325901 (1 μM), IWP-2 (2 μM), SB590885 (0.5 μM). VPA (500 μM), Tranylcypromine (10 μM), DZNep (0.05 μM), and EPZ004777 (5 μM) were included in the first 4 days. In several experiments, to testing the reprogramming efficiency after removing each small molecules, the indicated small molecules were removed from the cocktails. The concentration of the small molecules used to targeting the same pathway or targets were indicated as follow: GSK3 inhibitors (CHIR99021: 0-10 μM); ERK inhibitors (PD0325901: 0.02-5 μM; AZD8330: 0.2-5 μM; TAK733: 0.2-5 μM; Tramitinib: 0.2-5 μM; U0126: 0.2-5 μM); Rock inhibitors (Y27632: 2-12 μM; TZV: 0.2-0.5 μM; Fasudil: 2-10 μM; HA-1100: 4-20 μM; Blebbistatin: 2-10 μM); WNT pathway inhibitors (IWP2: 0.5-4 μM; IWR1: 1-10 μM; XAV939: 1-10 μM); BRAF inhibitors (SB590885: 0.1-5 μM; Sorafinib: 0.1-0.5 μM; GDC0879: 0.1-5 μM); HDAC inhibitors (VPA: 200-1000 μM; MS275: 0.2-1 μM; Butyrate sodium: 100-500 μM).

Induction Process of hCiPSCs From hADSCs and hASFs

Hypoxia with 5% O₂ was applied in stage I induction. After stage I induction, cells were changed into 21% O₂. The induction medium was changed every 3-4 days. (1). ADSCs and hASFs were seeded at a density of 1×10⁴ cells per well of a 12-well plate in 15% FBS DMEM medium. Change the medium into stage I induction medium on the next day. (2). For stage I induction, single layer epithelial-like cells induced from hADSCs would emerge at day 4-6 and approach 80%-100% confluence at day 8-12. For ASFs, epithelial-like cells would approach 80%-100% confluence at day 12-20. Then change the medium into stage II induction medium. (3) For stage II induction, multi-layered cell colonies appeared after 8-12 days treatment and these cell colonies would continually grow larger. After totally 16-20 days' treatment of stage II medium, change the medium into stage III induction medium. (4) For stage III induction, 10-12 days' treatment of stage III induction medium was required. Then change the medium into stage IV condition. (5) For stage IV induction, VPA (500 μM), Tranylcypromine (10 μM), DZNep (0.05 μM), and EPZ004777 (5 μM) were included in the first 4 days of stage IV induction medium. Primary hCiPSC colonies would emerge after 6-8 days' treatment.

### Derivation and Culture of Human CiPS Cell Lines

After 8-12 days stage IV condition treatment, cells were dissociated by Accutase (Millipore, SCR005) and replated at a ratio from 1:3 to 1:12 on feeder layers of mitomycin C (Sigma-Aldrich, M4287)-treated MEFs (2-3×10⁴ per cm²) in the modified stage IV condition: Knockout DMEM supplemented with 1% N2 supplement, 2% B27 supplement, 1% GlutaMAX™, 1% NEAA, 1% Penicillin-Streptomycin, 0.055 mM 2-mercaptoethanol, 2 mg/mL AlbuMAX™-II and the small molecules CHIR99021 (1 μM), PD0325901 (0.5 μM), IWP-2 (2 μM), Y-27632 (10 μM), HRG (20 ng/ml), and bFGF (100 ng/ml, Origene). Cells were incubated under 21% O₂, 5% CO₂ at 37° C. and the medium was changed every day. After 3-7 days, compact CiPS cell colonies appeared. After 10-12 days, these colonies were manually picked up and mechanically dissociated into small clamps and transferred onto Matrigel (Corning, 354248) coated plates in mTeSR™ Plus Medium (STEMCELL, 05826) supplemented with Y-27632 (10 μM). Allow the colonies to attach to the culture plate for 24 hours before replacing the spent medium with fresh mTeSR™ Plus Medium without Y-27632.

Human CiPS cells and hES cells (H1 and H9) were maintained in mTeSR™ Plus Medium on Matrigel coated plates under 21% O₂, 5% CO₂ at 37° C. The medium was changed every day. Cells were passaged when they reach ˜85% confluence. This typically occurred at day 3-7 after passaging with split ratios of around 1:10 to 1:20. For passaging, human CiPS cells were dissociated by ReLeSR™ (STEMCELL, 05872), and the detached cell aggregates were transferred onto Matrigel-coated plates in mTeSR™ Plus Medium supplemented with Y-27632 (10 μM). Allow the colonies to attach to the culture plate for 24 hours before replacing the spent medium with fresh mTeSR™ Plus Medium without Y-27632.

### Immunofluorescence

Immunofluorescence was performed as previously described (Hou et al., 2013). After fixation with 4% paraformaldehyde (DingGuo, AR-0211) at room temperature for 30 min, cells were permeabilized and blocked with PBS containing 0.1% Triton™ X-100 (Sigma-Aldrich, T8787) and 2% donkey serum (Jackson Immuno Research, 017-000-121) at 37° C. for 1 hour. Primary antibodies incubation with appropriate dilutions were performed at 4° C. overnight in the same buffer. On the next day, cells were washed with PBS for three times and probed with secondary antibodies in PBS containing 2% donkey serum at 4° C. overnight. Cells were then washed with PBS for three times and DNA was stained with DAPI solution (Roche Life Science, 10236276001). Antibody details are provided in the Table 1.

TABLE 1
Antibody information

| Antibodies | Company | Cat. NO. | Dilution |
|---|---|---|---|
| LIN28A | Invitrogen | PA1-096 | 1:200 |
| SALL4 | Abcam | ab57571 | 1:200 |
| FOXA2 | Cell Signaling | 8186S | 1:200 |
| | Technology | | |
| SOX17 | R&D | AF1924 | 1:200 |
| EpCAM | Invitrogen | MA5-12436 | 1:200 |
| OCT4 | BD Biosciences | 611203 | 1:200 |
| OCT4 | Invitrogen | MA5-14845 | 1:200 |
| SOX2 | R&D | AF2018 | 1:200 |
| NANOG | Abcam | ab21624 | 1:200 |
| TRA-1-60 | Millipore | MAB4360 | 1:100 |
| TRA-1-81 | Millipore | MAB4381 | 1:100 |
| SSEA-4 | Santa Cruz | sc-21704 | 1:100 |
| GATA4 | R&D | AF2606 | 1:400 |
| T | R&D | AF2085 | 1:200 |
| SOX1 | R&D | AF3369 | 1:200 |
| TUJ1 | BioLegend | 801201 | 1:200 |
| 7-AAD | BD Pharmingen | 559925 | 1:1500 |
| anti-human CD144 | BioLegend | 348516 | 1:1500 |
| anti-human CD31 | BioLegend | 303106 | 1:1500 |
| anti-human CD34 | BioLegend | 343504 | 1:1500 |
| anti-human CD5 | BioLegend | 300608 | 1:1500 |
| anti-human CD4 | BioLegend | 300514 | 1:1500 |
| CD8b Monoclonal | Invitrogen | SIDI8BEE | 1:1500 |
| Antibody | | | |

### Population Doubling Time

The growth rate was determined by counting the number of cells using a hemocytometer as a function of time. Data from the exponential phase of growth were used. The doubling time was calculated following the formula: DT=t*[lg2/(lgNt−lgNo)].

### Teratoma Formation

For teratoma formation, the hCiPS cells were harvested by ReLeSR™. Approximately 2×10⁶ cells were resuspended in Matrigel and then sub-cutaneously injected to the immunodeficient NPG mice. Teratomas generally obtained by 6-7 weeks, and then embedded in paraffin. The paraffin sections were stained by hematoxylin and eosin. All of the animal experiments were performed according to the Animal Protection Guidelines of Peking University, China.

### EB Formation

hCiPSCs were harvested as small clumps and seeded as spheres on ultra-low attachment plates in mTESR™ Plus Medium for 1 day to form embryoid bodies, and differentiated in high glucose DMEM supplemented with 20% FBS for 16 days. Then, EBs were collected and plated on the Matrigel-coated plates for 6 days in the same medium, fixed and detected with immunostaining.

### Directed Differentiation

Hematopoietic and T Cell Differentiation of hCiPSCs

Mesoderm and hematopoietic endothelial (HE) cell differentiation from pluripotent stem cells were induced as previously described with optimizing conditions (Wang et al., 2012). Briefly, pluripotent stem cells were cultured in Matrigel-coated plate with low density from 1×10⁴˜5×10⁴ per well in 6-well plate day 1 before the differentiation. At differentiation day 0, RPMI 1640 (Gibco, 61870036) supplemented with B27 without vitamin A, 20 ng/mL Activin A, 20 ng/mL BMP4 (StemImmune LLC, HST-B4-0100), 50 μg/ml Vc2p, 3-5 μM CHIR99021, 1% GlutaMAX™, 1% NEAA, 1% Penicillin-Streptomycin and 0.1 mM 1-thioglycerol (Sigma, M6145) were administrated for 2 days. After 2 days of mesodermal induction, the differentiated EBs were cultured with RPMI 1640 supplemented with B27 without vitamin A, 50 μg/ml Vc2p, 5 ng/ml BMP4, 50 ng/ml VEGF (StemImmune LLC, HVG-VF5-1000), 50 ng/ml bFGF (Origene, TP750002), 1% GlutaMAX™, 1% NEAA, 1% Penicillin-Streptomycin and 10 μM SB-431542 (Selleck, S1067). Next, for hematopoietic induction, the culture medium was changed on day 6 to IMDM (Gibco, 12440053) supplemented with B27 without vitamin A, 50 μg/ml Vc2p, 5 ng/ml BMP4, 10 ng/ml VEGF, 20 ng/ml SCF (StemImmune LLC, HHM-SF-1000), 1% GlutaMAX™M, 1% NEAA, 1% Penicillin-Streptomycin. On day 8, hematopoietic progenitor cells were collected and transferred onto MS5-DL4 cells and co-cultured in T cell differentiation medium (IMDM supplemented with B27 without vitamin A, 50 μg/ml Vc2p, 1% GlutaMAX™M, 1% NEAA, 1% Penicillin-Streptomycin, 0.1 mM 1-thioglycerol, 5 ng/ml SCF, 5 ng/ml FLT3 (StemImmune LLC, HHM-FT-1000), 5 ng/ml IL7 (StemImmune LLC, HCT-17-1000)). Medium was changed every 2 days. For surface marker detection, cultured cells were collected and the indicated antibodies were added. Flow cytometry analysis was conducted using FACSVerse (BD). The data were analyzed using FlowJo-V10 (BD).

Hepatocytes Differentiation of hCiPSCs

Hepatocytes differentiation from pluripotent stem cells were induced as previously described (Chen et al.,2020).Briefly, hCiPSCs were induced into primitive streak with the combination of 100 ng/ml Activin A, 0.5 ng/ml BMP4, 10 ng/ml bFGF (PEPROTECH, 100-18B) and 20 ng/ml Wnt3a for 1 day in RPMI 1640 medium with B27 supplement and 1% Penicillin-Streptomycin, and were induced into definitive endoderm cells with the combination of 100 ng/ml Activin A, 0.5 ng/ml BMP4 and 10 ng/ml bFGF for 3 days. The hCiPSC-derived endoderm cells were further specified into foregut endoderm cells with the combination of 20 ng/ml KGF (StemImmune, EST-KF-1000) and 5 μM SB-431542 for 2 days in RPM I1640 medium with B27 supplement and 1% Penicillin-Streptomycin. hCiPSC-derived foregut endoderm cells were then induced to differentiate into hepatoblasts with the combination of 20 ng/ml KGF, 20 ng/ml BMP4, 10 ng/ml BMP2 (Stemimmune, HST-B2-1000) and 10 ng/ml bFGF for another 3 days in RPMI 1640 medium with B27 supplement and1% Penicillin-Streptomycin. The hCiPSC-derived hHPCs were induced to differentiate into mature hepatocytes with the hHPC maturation medium: Williams' E medium with B27 supplement, 25 μM Forskolin and 10 μM SB-431542. Lipid detection was performed with a Lipid (Oil Red O) Staining Kit (Sigma, MAK194) according to the manufacturer's instructions. Human albumin was measured using the Human Albumin ELISA Quantitation kit (Bethyl Laboratory, E80-129) according to the manufacturer's instructions. Urea synthesis was measured using the QuantiChrom Urea Assay Kit (BioAssay System, BA_DIUR-500) according to the manufacturer's instructions.

### Karyotype Analysis

The Karyotype (chromosomal G-band) analyses were contracted out at Beijing Jiaen Hospital, using standard protocols for high-resolution G-banding (400G-500G) and analyzed by Cyto Vision (Leica). For each analysis, at least 20 metaphases were examined. The number of chromosomes as well as the presence of structural chromosomal abnormalities was examined.

### Short Tandem Repeat (STR) Analysis

Short-tandem repeat analyses were contracted out at Beijing Microread Genetics Co., Ltd. In brief, the genomic DNA was used for PCR with STR Multi-amplification Kit (Microreader™21 ID System) and analyzed by ABI 3730×1 DNA Analyzer (Applied Biosystems®) and GeneMapperID-X software.

Reverse Transcription (RT)-Quantitative PCR (qPCR)

Total RNA was isolated using Direct-zol RNA MiniPrep Kit (Zymo Research, R2053). cDNA was synthesized from 0.5-1 μg of total RNA using TransScript First-Strand cDNA Synthesis SuperMix (TransGen Biotech, AT311-03). qPCR was performed by using KAPA SYBR FAST qPCR Kit Master Mix (KAPA Biosystems, KM4101) on a CFX ConnectTM Real-Time System (Bio-Rad). The data were analyzed using the delta-delta Ct method. GAPDH was used as a control to normalize the expression of target genes. Primer sequences for qPCR in this study are listed in Table 2.

TABLE 2
Primers used for qRT-PCR

| Gene | Forward sequence (5′→3′) | Reverse sequence (5′→3′) |
|---|---|---|
| GAPDH | TGACATCAAGAAGGTGGTGAA | GCGTCAAAGGTGGAGGAGTG |
| | GCAGG (SEQ ID NO: 1) | GGT (SEQ ID NO: 2) |
| OCT4 | CTGGGTTGATCCTCGGACCT | CCATCGGAGTTGCTCTCCA |
| | (SEQ ID NO: 3) | (SEQ ID NO: 4) |
| NANOG | GATTTGTGGGCCTGAAGAAA | CAGATCCATGGAGGAAGGAA |
| | (SEQ ID NO: 5) | (SEQ ID NO: 6) |
| SOX2 | TTGCGTGAGTGTGGATGGGAT | GGGAAATGGGAGGGGTGCAA |
| | TGGTG (SEQ ID NO: 7) | AAGAGG (SEQ ID NO: 8) |
| DNMT3B | CGAAGAAGAGCCGGCCTGTAC | CGGAAGCCCATGCAACGATCT |
| | C (SEQ ID NO: 9) | C (SEQ ID NO: 10) |
| DPPA4 | GCTAACATCTGCCACCCCACC | GGATTCTGCGGTGCTGCTGAC |
| | A (SEQ ID NO: 11) | A (SEQ ID NO: 12) |
| UTF1 | CGCCGCTACAAGTTCCTTAAA | GGATCTGCTCGTCGAAGGG |
| | (SEQ ID NO: 13) | (SEQ ID NO: 14) |
| ZFP42 | AGAAACGGGCAAAGACAAGA | GCTGACAGGTTCTATTTCCGC |
| | C (SEQ ID NO: 15) | (SEQ ID NO: 16) |
| PRDM14 | TTGAGGAAGAGAATCAGATCC | CGTTCTGTACGGGGTCACTC |
| | AG (SEQ ID NO: 17) | (SEQ ID NO: 18) |
| ZIC3 | CAGGAGCTGTCGTGCAAGT | AGTAGCAGACGTGGTTGTTCT |
| | (SEQ ID NO: 19) | (SEQ ID NO: 20) |
| LIN28A | TTTCCCTCATTCCTGAACTGC | CAGCAAAATCAACCATCAAA |
| | (SEQ ID NO: 21) | TAAAC (SEQ ID NO: 22) |
| SALL4 | AGCACATCAACTCGGAGGAG | CATTCCCTGGGTGGTTCACTG |
| | (SEQ ID NO: 23) | (SEQ ID NO: 24) |
| KRT8 | CAGAAGTCCTACAAGGTGTCC | CTCTGGTTGACCGTAACTGCG |
| | A (SEQ ID NO: 25) | (SEQ ID NO: 26) |
| KRT18 | TCGCAAATACTGTGGACAATG | GCAGTCGTGTGATATTGGTGT |
| | C (SEQ ID NO: 27) | (SEQ ID NO: 28) |
| KRT19 | ACCAAGTTTGAGACGGAACAG | CCCTCAGCGTACTGATTTCCT |
| | (SEQ ID NO: 29) | (SEQ ID NO: 30) |
| SOX17 | AGGAAATCCTCAGACTCCTGG | CCCAAACTGTTCAAGTGGCAG |
| | GTT (SEQ ID NO: 31) | ACA (SEQ ID NO: 32) |
| APOA1 | CCCTGGGATCGAGTGAAGGA | CTGGGACACATAGTCTCTGCC |
| | (SEQ ID NO: 33) | (SEQ ID NO: 34) |
| APOA2 | CTGTGCTACTCCTCACCATCT | CTCTCCACACATGGCTCCTTT |
| | (SEQ ID NO: 35) | (SEQ ID NO: 36) |
| FOXA2 | CCTACGAACAGGTGATGCACT | GATTTCTTCTCCCTTGCGTCT |
| | (SEQ ID NO: 37) | (SEQ ID NO: 38) |
| GATA6 | CTCAGTTCCTACGCTTCGCAT | GTCGAGGTCAGTGAACAGCA |
| | (SEQ ID NO: 39) | (SEQ ID NO: 40) |
| HNF1B | GCACCTCTCCCAGCATCTCA | GTCGGAGGATCTCTCGTTGC |
| | (SEQ ID NO: 41) | (SEQ ID NO: 42) |
| MMP1 | AAAATTACACGCCAGATTTGC | GGTGTGACATTACTCCAGAGT |
| | C (SEQ ID NO: 43) | TG (SEQ ID NO: 44) |
| PRRX1 | AAGTAGCCATGGCGCTGTA | GAGACGTGACTGCTGTGGAG |
| | (SEQ ID NO: 45) | (SEQ ID NO: 46) |
| SNAI2 | CTGAGGATCTCTGGTTGTGGT | CGAACTGGACACACATACAG |
| | (SEQ ID NO: 47) | TG (SEQ ID NO: 48) |
| TWIST1 | CGGGAGTCCGCAGTCTTA (SEQ | GCTTGAGGGTCTGAATCTTG |
| | ID NO: 50) | (SEQ ID NO: 51) |
| TWIST2 | CTTATGTTTGGGGGGAGGTT | TAGCCAAGCAATCACGGAGA |
| | (SEQ ID NO: 52) | (SEQ ID NO: 53) |
| VIM | CTTTGTCGTTGGTTAGCTGGT | GACGCCATCAACACCGAGTT |
| | (SEQ ID NO: 54) | (SEQ ID NO: 55) |
| ZEB1 | ATCAGCCTAACCATACAACTC | CAGGAACAAATTGGCACAA |
| | T (SEQ ID NO: 56) | (SEQ ID NO: 57) |
| ZEB2 | GGGTTCTTTCATTTGTTTTGGT | ACTCCAAACAGCTTCTCTTCT |
| | (SEQ ID NO: 58) | GA (SEQ ID NO: 59) |
| COL1A1 | CAGATCACGTCATCGCACAAC | GAGGGCCAAGACGAAGACAT |
| | (SEQ ID NO: 60) | C (SEQ ID NO: 61) |
| COL5A1 | GCCCGGATGTCGCTTACAG | AAATGCAGACGCAGGGTACA |
| | (SEQ ID NO: 62) | G (SEQ ID NO: 63) |
| COL6A2 | GACTCCACCGAGATCGACCA | CTTGTAGCACTCTCCGTAGGC |
| | (SEQ ID NO: 64) | (SEQ ID NO: 65) |
| ALB | GCACAGAATCCTTGGTGAACA | ATGGAAGGTGAATGTTTCAGC |
| | G (SEQ ID NO: 66) | A (SEQ ID NO: 67) |
| ASL | CAGTGGACCCCATCATGGAGA | GGCTTTGCTGCCTTGAACATC |
| | (SEQ ID NO: 68) | (SEQ ID NO: 69) |
| ASS | CTTGGGGCCAAAAAGGTGTTC | GAGGTAGCGGTCCTCATACAG |
| | (SEQ ID NO: 70) | (SEQ ID NO: 71) |
| CPS1 | AATGAGGTGGGCTTAAAGCAA | AGTTCCACTCCACAGTTCAGA |
| | G (SEQ ID NO: 72) | (SEQ ID NO: 73) |
| OTC | CGGCCCGTGTATTGTCTAGC | TAGCCAGGGTGTCCAAATCTG |
| | (SEQ ID NO: 74) | (SEQ ID NO: 75) |
| CYP3A4 | GGTGGTGAATGAAACGCTCAG | ACCCCTTTGGGAATGAACATC |
| | (SEQ ID NO: 76) | (SEQ ID NO: 77) |
| F10 | CACTGGTCGCCATCTTTGTA | AGTGCATGGAAGAGACCTGC |
| | (SEQ ID NO: 78) | (SEQ ID NO: 79) |
| FROX1 | ACAGGGCTCTGAACATGCAC | GGCATTGAAAAACTCCCGTA |
| | (SEQ ID NO: 80) | (SEQ ID NO: 81) |
| HNF4A | ACTACATCAACGACCGCCAGT | ATCTGCTCGATCATCTGCCAG |
| | (SEQ ID NO: 82) | (SEQ ID NO: 83) |

### RNA Sequencing

Total RNA was isolated using Direct-zol RNA MiniPrep Kit. RNA sequencing libraries were constructed using the NEBNext Ultra RNA Library Prep Kit for Illumina (NEB England BioLabs, E7775). Fragmented and randomly primed 2 ×150 bp pairedend libraries were sequenced using Illumina HiSeq X Ten

Single-Cell RNA Sequencing (scRNA-seq)

Using single cell 3′ Library and Gel Bead Kit V3.1 (10x Genomics, 1000075) and Chromium Single Cell B Chip Kit (10x Genomics, 1000074), the cell suspension (300-600 living cells per microliter determined by Count Star) was loaded onto the Chromium single cell controller (10x Genomics) to generate single-cell gel beads in the emulsion according to the manufacturer's protocol. In short, cells at different time points throughout chemical reprogramming process were harvested and resuspended at 1×10⁶ cells per milliliter in 1×PBS with 0.04% BSA. About 1×10⁴ cells were added to each channel, and the target cell will be recovered was estimated to be about 5×10³ cells. Captured cells were lysed and the released RNA were barcoded through reverse transcription in individual GEMs. Reverse transcription was performed on a S1000TM Touch Thermal Cycler (Bio Rad) at 53° C. for 45 min, followed by 85° C. for 5 min, and hold at 4° C. The cDNA was generated and then amplified, and quality assessed using an Agilent 4200. According to the manufacture's introduction, Single-cell RNA-seq libraries were constructed using Single Cell 3′ Library and Gel Bead Kit V3.1 The libraries were finally sequenced using an IlluminaNovaseq6000 sequencer with a sequencing depth of at least 1×10⁵ reads per cell with pair-end 150 bp (PE150) reading strategy (performed by CapitalBio Technology, Beijing).

### Assay for Transposase-Accessible Chromatin Using Sequencing (ATAC-seq)

Approximately 5×10⁴ cells of each sample were collected and washed once with cold PBS and re-suspended in 50 μL Lysis buffer (10 mM Tris-HCl PH7.4, 10 mM NaCl, 3 mM MgCl₂, 0.5% NP-40). Following DNA library construction were prepared by TurePrep DNA Library Prep Kit V2 for Illumina (Vazyme, TD501-02) and amplified by PCR using TruePrep Index Kit V2 for Illumina (Vazyme, TD202 96rxn). All ATAC libraries were sequenced on an Illumina Novaseq 6000 platform and 150 bp paired-end reads were generated. Biologically independent experiments n=3.

### Whole-Genome Bisulfite Sequencing (WGBS)

Genomic DNA was isolated from HEFs, hADSCs, hCiPSCs and hESCs. Bisulfite conversion of the extracted DNAs were performed as previously reported (2018, Zhao et al.). The recovered bisulfite-converted DNAs were constructed into sequencing libraries and each library was sequenced 90G raw data by Illumina HiSeq X Ten.

### Bisulfite Genomic Sequencing

DNA was extracted using Quick-DNA Miniprep kit (Zymo Research, D3024). The isolated DNA was modified by bisulfite treatment and purified using EZ DNA Methylation-Direct Kit (Zymo Research, D5020). Then the bisulfite-modified DNA was amplified by PCR using ZymoTaq PreMix kit (Zymo Research, E2003). The primers are listed in Table 2. The amplified fragments were cloned into the pEASY-T1 Simple Cloning vector (Transgen, CT111-02). Ten randomly picked clones from each sample were sequenced.

### RNA-Seq Analysis

Quality control was performed using FastQC to all samples. The raw RNA-seq reads were trimmed using Trimmomatic with parameter ‘ILLUMINACLIP:TruSeq3-PE-2.fa:2:30:7:1:true LEADING:3 TRAILING:3 SLIDINGWINDOW:4:15 MINLEN:36’ to remove detected adapters. The clean reads to human reference genome hg19 were mapped using STAR with additional parameter ‘—outSAMtype BAM Unsorted—outSAMstrandField intronMotif—outFilterIntronMotifs RemoveNoncanonical’. The number of reads mapped to each gene was counted using program featureCounts of Subread package.

Differentially expressed gene analysis were performed using R package DESeq2. In detail, genes in the lowest 40% quantile of mean expression were removed, and preprocess were performed using DESeq function with default parameters. The gene expression differences between groups was calculated using lfcShrink with adaptive t prior shrinkage estimator apeglm. The differentially expressed genes (DEG) were defined as the genes with log2 fold change>2 between groups and adjusted p value<0.05. GO analysis to DEGs was performed using function enrichGO of R package clusterProfiler. The normalized counts were also variance stabilizing transformed and scaled to plot heatmap. Principle Component Analysis (PCA) was performed using R package irlba with parameters ‘scale.=T, n=3’.

### WGBS Analysis

To perform alignments of bisulfite-treated reads correctly, the Bismark Bisulfite Mapper pipeline was followed. First, the human reference genome hg19 and lambda genome were transformed into fully bisulfite-converted forward (C>T) and reverse (G>A conversion of the forward strand) versions using the script bismark_genome_preparation. Sequence reads were similarly transformed and mapped to prepared genomes using Bowtie2 with parameters set by Bismark. Then the alignments to the same position in the genome from the Bismark mapping output were removed using the script deduplicate_bismark. The methylation information of every single C was extracted using the script bismark_methylation_extractor with parameter ‘-p—no_overlap—ignore 5—ignore_r2 5—zero_based—CX—buffer_size 50%’. In this way, the number of reads were obtained that supported methylation and the number of reads covering every CpG site, non-CpG sites were not considered in later analysis.

To identify the sites showing evidence of methylation, binomial test was performed to each site on these counts to test whether the methylation counts are beyond bisulfite non-conversion events. The bisulfite non-conversion rate was defined as the total methylation level of the lambda genome which was spiked in during library construction. The false discovery rate (FDR) was computed using Benjamini-Hochberg method. The sites whose FDR above 0.01 were regarded as non-conversion sites and their methylation levels were set to 0. Methylation level for each CpG site passed the test was defined as the fraction of reads supporting methylation out of reads that cover the site (mCpG/CpG). For regions with multiple CpG sites, methylation levels were defined as weighted methylation (total mCpG/total CpG). The mean CpG methylation level of each sample was calculated as the fraction of all reads supporting CpG methylation out of reads that cover CpG site (all mCpG / all CpG).

To compare the global similarity of different samples, the genome was cut to bins of length 50000 bp and calculated the CpG methylation level of each bin. These methylation levels were used as the estimation of global CpG methylation feature. Hierarchical clustering was performed using function hclust with 1-Pearson correlation coefficient as distant parameter.

### ATAC-Seq Analysis

Quality control was performed using FastQC to all samples. The raw ATAC-seq reads were trimmed using Trimmomatic with parameter ‘ILLUMINACLIP:NexteraPE-PE.fa:2:30:7:1:true LEADING:3 TRAILING:3 SLIDINGWINDOW:4:15 MINLEN:36’ to remove detected adapters. Then the clean reads were mapped to human reference genome hg19 using Bowtie2 with parameter ‘—very-sensitive’. The output SAM files were converted to BAM files and sorted by name using Samtools. Duplicated reads were removed using MarkDuplicates function of Picard tools. Low quality mapped reads and reads mapped to mitochondrial were filtered using Samtools. After adjusting fragments 5bp forward to account for the Tn5 transposase occupancy peak calling was performed to each sample using MACS2 with parameters ‘-g hs—nomodel—shift-100—extsize 200—keep-dup all’. To keep only consensus peaks among replicates of the same sample, Irreproducible Discovery Rate (IDR) analysis was performed between each pair of replicates, only peaks with score>540 in at least one test were preserved.

Overlapped peaks in different samples were merged and defined sample specific peak sets using R package ChIPpeakAnno. Each peak was then annotated to a closest gene using R package ChIPseeker. The ATAC signal strength around peak region or gene promoter region was quantified using Deeptools.

### Single-Cell RNA-Seq Data Preprocess

The clean single-cell RNA-seq reads were mapped to human reference genome hg19 using Cellranger v3.1.0. Cells less than 500 total gene numbers or less than 1000 total UMI counts were removed. Then the UMI count proportion of mitochondrial genes (M %) of each cell was checked and cells whose M>median+2MADs (median absolute deviation) or M<median−2MADs after log scale were removed. The cells passed quality control were roughly clustered based of their Spearman's rank correlation using function quickCluster of R package scran with parameter ‘use.ranks=T’. Next scaling normalization was performed by deconvolving size factors from cell pools using function computeSumFactors and logNormCounts with parameter ‘downsample=T, down_prop=0.1’ The normalized data was used to create a Seurat object using R package Seurat v3. PCA dimensionality reduction was performed using 2000 variable features and scaled normalized data. Then the Uniform Manifold Approximation and Projection (UMAP) dimensionality reduction was performed using the first 20 PCs. A Shared Nearest Neighbor (SNN) graph was constructed using the first 20 PCs and identified clusters of cells based on the SNN graph using function FindClusters with resolution 0.6. The Adaptively-thresholded Low Rank Approximation (ALRA) imputed normalized data calculated by function RunALRA was used to visualize the gene expression value.

### Transcriptome Analysis of XEN-like Cells With Reported Datasets

XEN-like clusters were identified in samples by some canonical markers. To compare these XEN-like cells with known XEN cells, the public dataset was used as reference: human pre-implantation embryo data (E-MTAB-3929, Petropoulos et al., 2016). Preprocess was performed to this dataset in a similar way, the UMI count matrix were normalized to TPM and performed ALRA imputation. The Pearson correlation coefficient of all cell types was calculated using 300 lineage markers defined by previous paper (Petropoulos et al., 2016). Gene Set Enrichment Analysis (GSEA) was performed using R package clusterProfiler.

### Statistical Analysis

For statistical analysis, p values were calculated through unpaired two-tailed Student's t-test using GraphPad Prism 8. p values are as follows: *p<0.05; **p<0.01; ***p<0.001; ****p<0.0001. The results are presented as mean+SD as indicated in the figure legends.

### RESULTS AND DISCUSSION

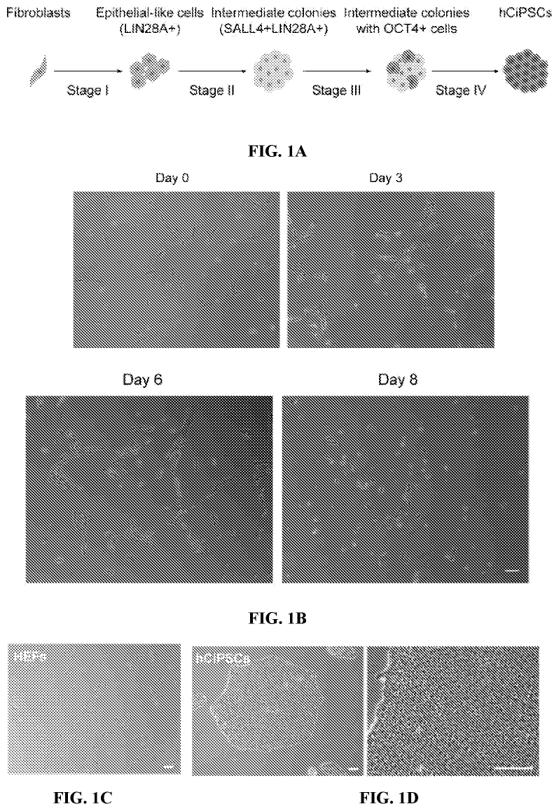

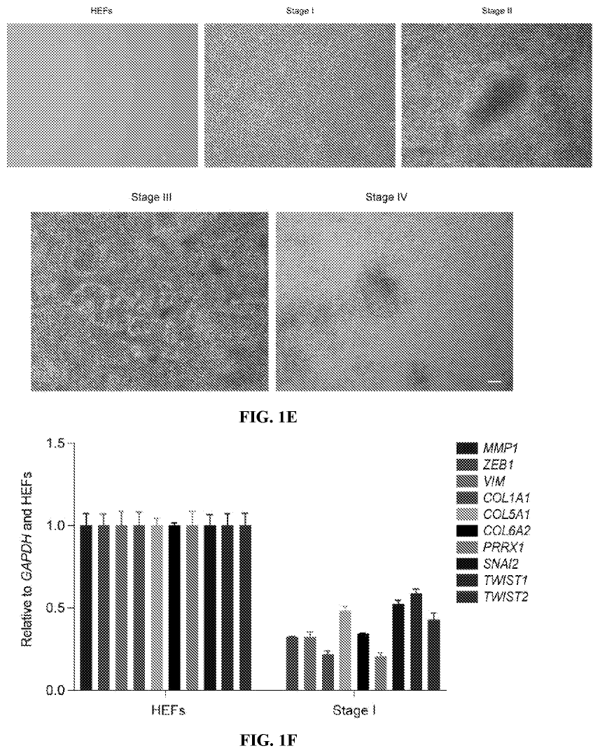

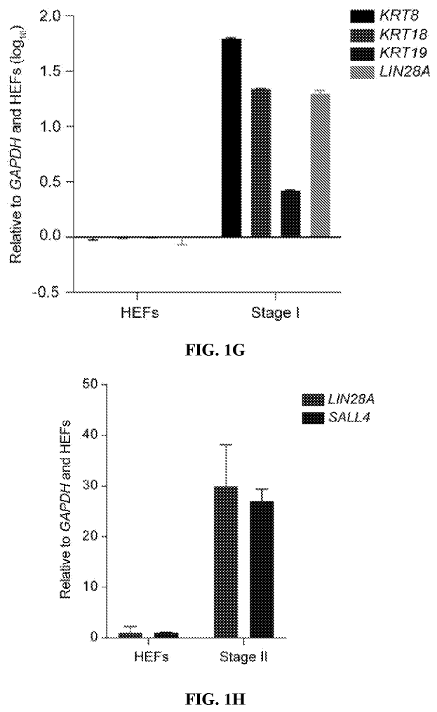

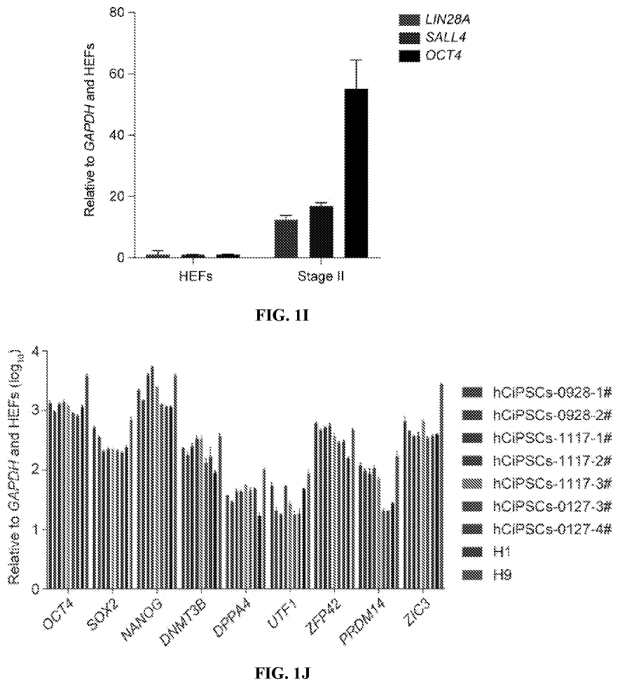

To induce human fibroblasts into pluripotent stem cells by small molecules, a previously disclosed chemical cocktail which successfully induced the mouse pluripotency (7-10) was first tested on human embryonic fibroblasts (HEFs). However, those HEFs proliferated poorly and showed apoptotic phenotypes after the treatment (FIG. 1B), consistent with previous findings that human cells, compared to mouse cells, were more refractory to exogenous signals (15). To meet the challenge of using exogenous signals to change human somatic cell fate, a strategy was adopted, that included small molecules with select biological activities, the combination of which was used to erase somatic gene program, activate pluripotency genes, and establish an integrated pluripotency network. Finally, a stepwise approach was established to reprogram human somatic cells to pluripotent stem cells (FIG. 1A).

To induce human fibroblasts into pluripotent stem cells by small molecules, a variety of small molecule cocktails were first tested on human embryonic fibroblasts (HEFs). However, at the initial stage, several major barriers were encountered: 1) the HEFs maintained their fibroblast morphology and didn't undergo mesenchymal to epithelial transition; 2) or the HEFs proliferated poorly and showed apoptotic phenotypes after the treatments; 3) it is also challenging to activate the expression of pluripotent related genes. These phenomena were consistent with previous findings that human cells which possessed stable epigenome (5-6) were refractory to exogenous signals (15). To meet the challenge of using exogenous signals to change human somatic cell fate, a strategy that combined chemical library screening and small molecule combinations to erase somatic gene program, activate pluripotency genes, and establish an integrated pluripotency was adopted network. Finally, a stepwise approach was established to reprogram human somatic cells to pluripotent stem cells. (FIG. 1A).

To disrupt the original cell identity and downregulate the somatic gene program, chemical screening was performed to identify a small molecule combination (CHIR99021, 616452 and TTNPB) that could convert human fibroblasts (FIG. 1C) into monolayer epithelial-like cells (FIG. 1E). Additional screening revealed that adding Y27632, ABT869 and SAG into the combination can facilitate the generation of the epithelial-like cells (stage I condition). After that treatment, a panel of fibroblast marker genes were downregulated (FIG. 1F) and epithelial cell-related genes, such as KRT8, KRT18, and KRT19, were upregulated (FIG. 1G). In addition, the pluripotency related gene LIN28A was expressed highly in the treated cells (FIG. 1B and FIG. 1G). However, at this stage other key pluripotency genes were not significantly activated. Subsequent screening on those stage I epithelial-like cells showed that after adding the epigenetic regulators 5-Azacytidine and Tranylcypromine combined with JNKIN8 to the stage I condition (stage II condition), the pluripotency-related transcriptional factor SALL4 was activated and co-expressed with LIN28A (FIG. 1B and FIG. 1H). However, the master pluripotency transcriptional factor OCT4, key to establishing the pluripotency network, was not activated in those cells.

OCT4 was activated by a combination of small molecules, including epigenetic regulators (Tranylcypromine, Valproic acid, DZNep, and EPZ004777) and signaling inhibitors (CHIR99021, 616452, Y27632, and PD0325901) (stage III condition) (FIG. 1B and FIG. 11). To further establish the fully pluripotent gene network, additional small molecules were added, to facilitate the maintenance of human pluripotent stem cells. After treatment with CHIR99021, PD0325901, SB590885, IWP2, and Y27632 (stage IV condition), compact colonies that co-expressed OCT4, SOX2, and NANOG (OSN) were generated (FIG. 1B). Importantly, after transferring into human embryonic stem cell (hESC) culture medium, those colonies displayed typical hESC morphology with tightly packed and high nuclear-to-cytoplasm ratio cells (FIG. 1D).

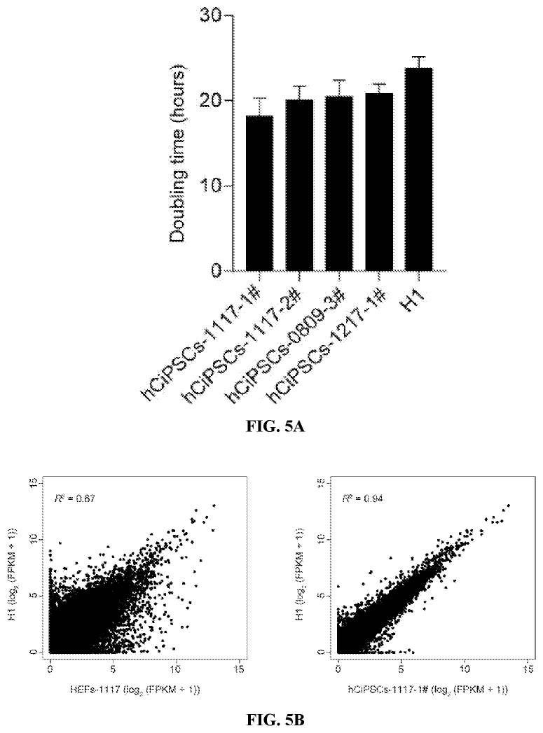

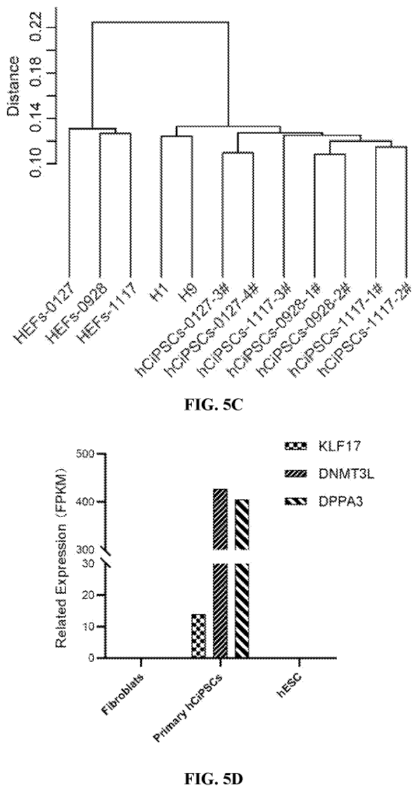

Then, gene expressions and epigenetic statuses of those established OSN cell lines were characterized. To begin, those cells, which can expand for more than 20 passages, proliferated with a doubling time similar to that of hESCs (FIG. 5A). They also expressed the surface markers TRA-1-60, TRA-1-81, and SSEA-4, along with the core pluripotency transcriptional factors OCT4, SOX2, and NANOG (FIG. 1E, and data not shown).

Additionally, RT-qPCR analysis showed the expression of pluripotency genes (OCT4 (octamer-binding transcription factor 4), SOX2 (SRY-Box Transcription Factor 2), NANOG (Nanog Homeobox), DNMT3B (DNA methyltransferase 3 beta), DPPA4 (developmental pluripotency-associated 4), UTF1 (undifferentiated embryonic cell transcription factor 1), ZFP42 (Zinc finger protein 42), PRDM14 (PR-domain containing protein 14) and ZIC3 (Zic Family Member 3)) in those cells, at levels comparable with that in hESCs (FIG. 1J) and RNA-sequencing analysis showed that the transcriptomes of those cells greatly resembled those of hESCs (FIG. 5B-C, and data not shown). The genes that highly expressed in hCiPSCs include: NANOG (Nanog Homeobox), PRDM14 (PR-domain containing protein 14), LIN28A (protein lin-28 homolog A), OCT4 (octamer-binding transcription factor 4), DPPA4 (developmental pluripotency-associated 4), EPCAM (Epithelial cell adhesion molecule), DNMT3B (DNA methyltransferase 3 beta), ZFP42 (zinc finger protein 42), SALL4 (Spalt Like Transcription Factor 4), ZIC3 (Zic Family Member 3), SOX2 (SRY-Box Transcription Factor 2), TDGF1 (teratocarcinoma-derived growth factor 1), DNMT3A (DNA methyltransferase 3 alpha), CDH1 (Cadherin 1), OTX2 (Orthodenticle Homeobox 2), ZIC2 (Zic Family Member 2). Also, the OCT4 and NANOG promoters had demethylation patterns and opened chromatin accessibility state like those of hESCs (data not shown). Additionally, the early passaged hCiPSCs several unique markers, such as Developmental Pluripotency Associated 3 (DPPA3), Kruppel-Like Factor 17 (KLF17) and DNA methyltransferase 3 like (DNMT3L). These markers were not expressed in the traditional pluripotent stem cells (hESCs and hiPSCs). Finally, the global modifications revealed by DNA methylation assays showed that those cells shared similar epigenomic status with hESCs (data not shown). Altogether, these results demonstrate that the transcriptomic and epigenetic profiles of OSN-positive cell lines resemble those of human pluripotent stem cells. Hereafter, the established OSN-positive cell lines are referred to as human chemically induced pluripotent stem cells (hCiPSCs).

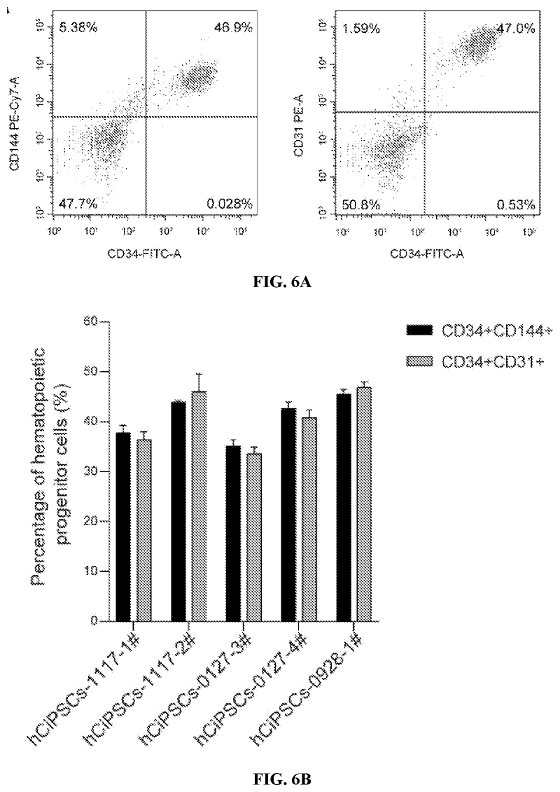

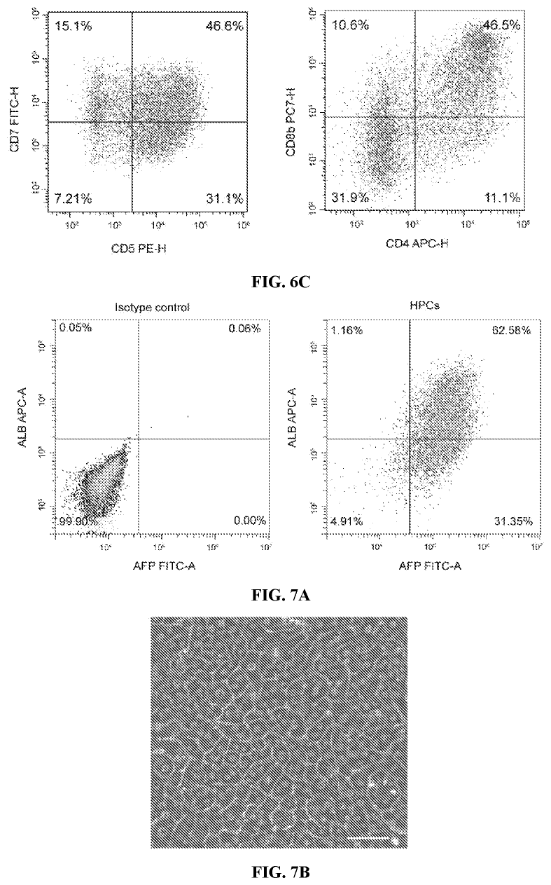

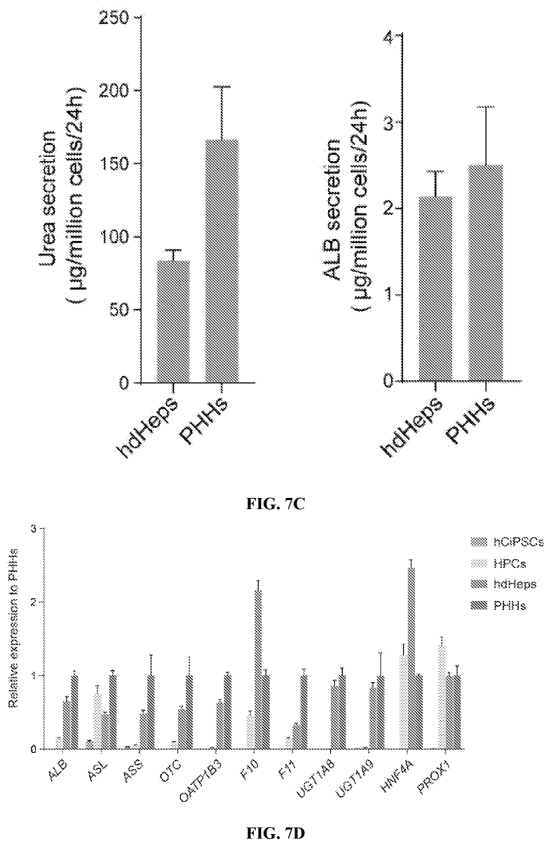

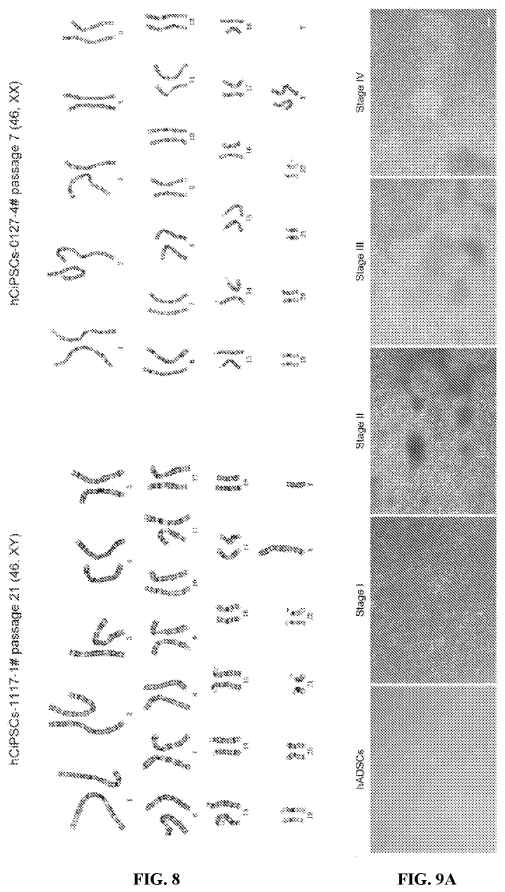

Next, the developmental potential of hCiPSCs in vivo and in vitro was characterized. First, hCiPSCs were injected into immunodeficient mice and the resultant teratomas contained tissues of all 3 germ layers (endo-, ecto-, and mesoderm) (data not shown). Consistent with this result, hCiPSCs formed embryoid bodies in vitro and expressed marker genes (FOXA2 (Forkhead box protein A2, SOX17 (SRY-box transcription factor 17, GATA4 (GATA Binding Protein 4), SOXI (SRY-Box Transcription Factor 1, T- (brachyury) and TUJI (Neuron-specific class III beta-tubulin)) of the three germ layers (data not shown). Directed differentiation demonstrated that hCiPSC lines could differentiate into hematopoietic progenitor cells (FIG. 6, A and B), and further differentiated toward T cell progenitors (FIG. 6C). In addition, hepatocytes were also generated by directed differentiation of hCiPSCs (FIG. 7A-D). Altogether, these results show that hCiPSCs' stable differentiation potency enables them to generate lineage-committed cells of all 3 germ layers. To assay hCiPSCs' genomic integrity, high-resolution G-banding analysis was used to karyotype the hCiPSC lines; the data showed that they all had normal diploid karyotypes (FIG. 8, and table 3).

TABLE 3
Karyotype analysis of hCiPS cell lines

| Human CiPS cell lines | Passage | Karyotype |
|---|---|---|
| hCiPSCs-0127-3# | P5 | Normal, 46, XX [20/20] |
| hCiPSCs-0127-4# | P7 | Normal, 46, XX [20/20] |
| hCiPSCs-1117-#1 | P7 | Normal, 46, XY [20/20] |
| hCiPSCs-1117-#1 | P21 | Normal, 46, XY [20/20] |
| hCiPSCs-1117-#1 | P41 | Normal, 46, XY [20/20] |
| hCiPSCs-1117-#2 | P6 | Normal, 46, XY [20/20] |
| hCiPSCs-1117-#2 | P22 | Normal, 46, XY [20/20] |
| hCiPSCs-1117-#2 | P42 | Normal, 46, XY [20/20] |
| hCiPSCs-1117-#3 | P9 | Normal, 46, XY [20/20] |
| hCiPSCs-1117-#3 | P22 | Normal, 46, XY [20/20] |
| hCiPSCs-0928-1# | P6 | Normal, 46, XY [20/20] |
| hCiPSCs-0928-2# | P6 | Normal, 46, XY [20/20] |
| hCiPSCs-0118-1# | P18 | Normal, 46, XX [20/20] |
| hCiPSCs-0118-2# | P21 | Normal, 46, XX [20/20] |
| hCiPSCs-0809-1# | P6 | Normal, 46, XX [20/20] |
| hCiPSCs-0809-3# | P6 | Normal, 46, XX [20/20] |
| hCiPSCs-1217-1# | P6 | Normal, 46, XY [20/20] |
| hCiPSCs-1217-2# | P6 | Normal, 46, XY [20/20] |
| hCiPSCs-1003# | P10 | Normal, 46, XY [20/20] |
| hCiPSCs-1230-4# | P6 | Normal, 46, XY [20/20] |
| hCiPSCs-1230-11# | P5 | Normal, 46, XY [20/20] |

Furthermore, short tandem repeat analysis confirmed that hCiPSCs were derived from their parental fibroblasts and distinct from other established hESC lines.

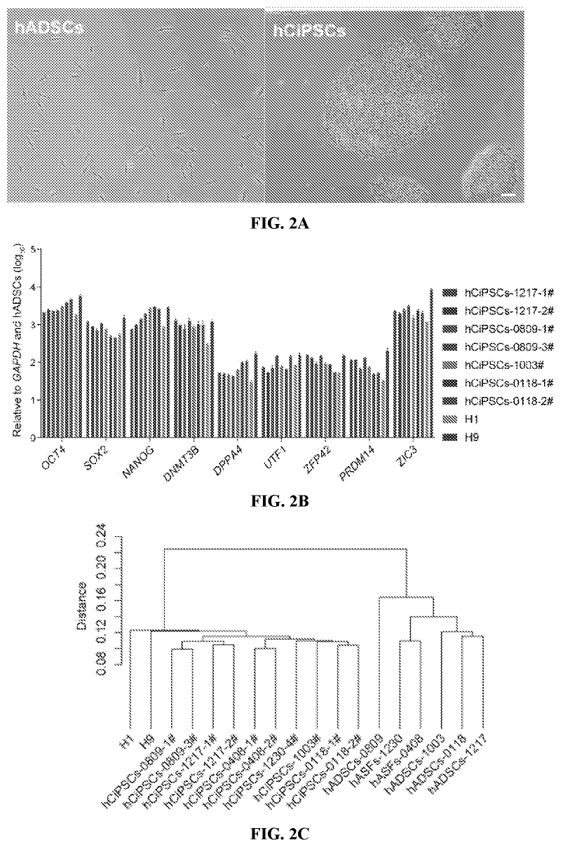

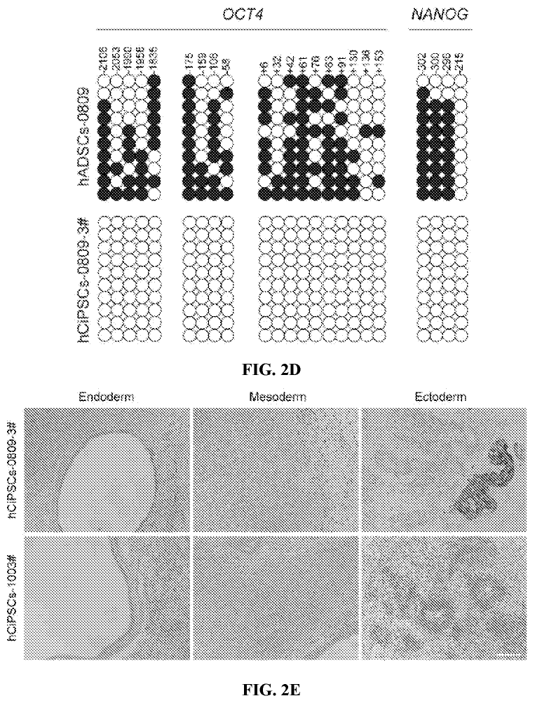

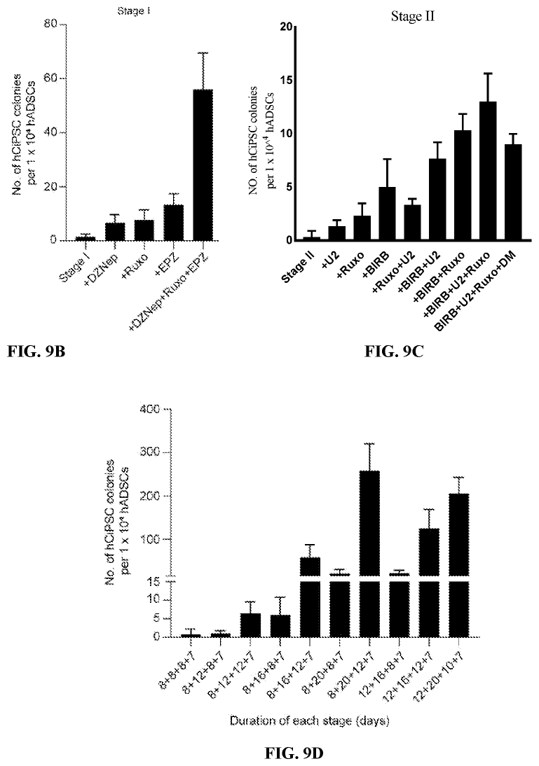

Studies were then conducted to chemically reprogram adult somatic cells to hCiPSCs. Following an additional screening, we identified facilitators that promote the reprogramming processes (FIG. 9A to C), and generated hCiPSCs from adult adipose-derived mesenchymal stromal cells (hADSCs) (FIG. 2, A and data now shown, and FIG. 9D). Furthermore, human adult skin dermal fibroblasts (hASFs) from different donors were reprogrammed to hCiPSCs (FIG. 11A). The transcriptomic and epigenetic profiles of adult somatic cell-derived hCiPSCs resembled those of human pluripotent stem cells (FIG. 2B to D, data not shown in FIG. 10A-B, and FIG. 11A-C and data not shown). Importantly, these adult somatic cell-derived hCiPSCs could also differentiate into cell types of all 3 germ layers, both in vivo and in vitro (FIG. 2E and data not shown). Moreover, the studies were 100% successful in reproducing hCiPSCs from the somatic cells of more than 8 independent donors (Table 4)

TABLE 4
Summary of reprogramming efficiencies of
hADSCs and hASFs from different donors.

| Cell line | Cell type | Age (years old) | Gender | Efficiency (%) |
|---|---|---|---|---|
| 0409 | hADSCs | 60 | Female | 2.56 ± 0.63 |
| 0509 | hADSCs | 38 | Female | 0.39 ± 0.09 |
| 0605 | hADSCs | 67 | Male | 0.21 ± 0.07 |
| 0618 | hADSCs | 63 | Female | 2.52 ± 0.33 |
| 0809 | hADSCs | 65 | Female | 1.59 ± 0.28 |
| 1217 | hADSCs | 63 | Male | 2.12 ± 0.76 |
| 1230 | hASFs | 46 | Male | 1.50 ± 0.42 |
| 0605 | hASFs | 67 | Male | 0.67 ± 0.42 |

hADSCs: human adult adipose-derived mesenchymal stromal cells
hASFs: human adult skin fibroblasts
Efficiency is calculated as the number of primary hCiPSC colonies divided by the number of input cells
Values are reported as mean ± SD (n >= 3)

These results demonstrate that our small molecule approach can reprogram different types of adult somatic cells into pluripotent stem cells.

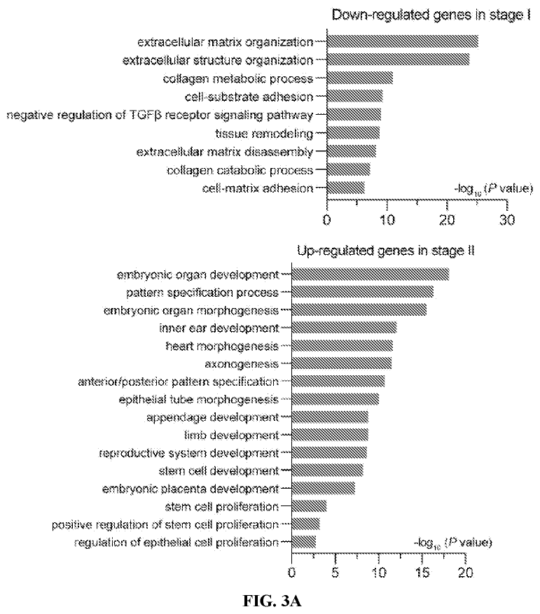

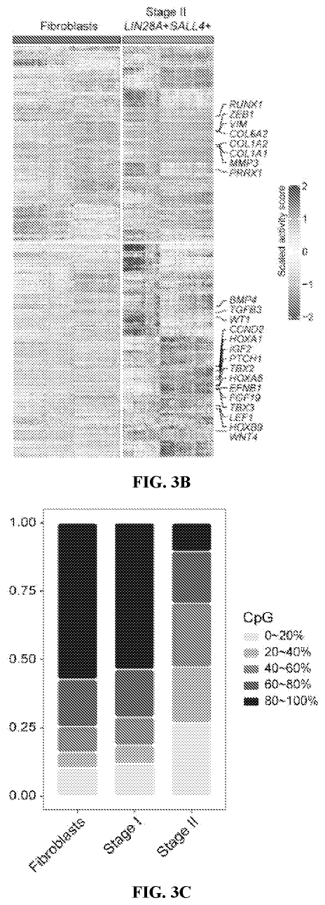

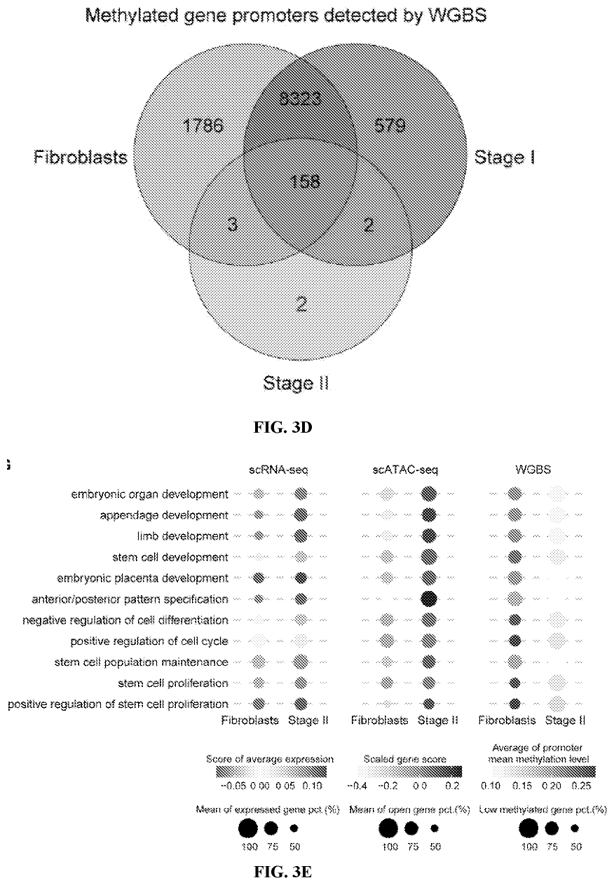

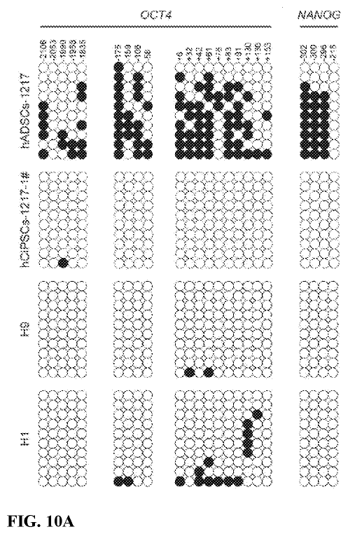

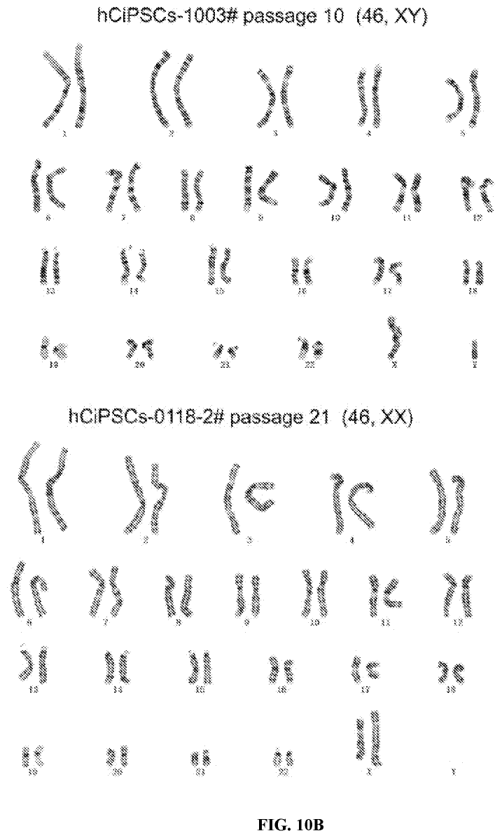

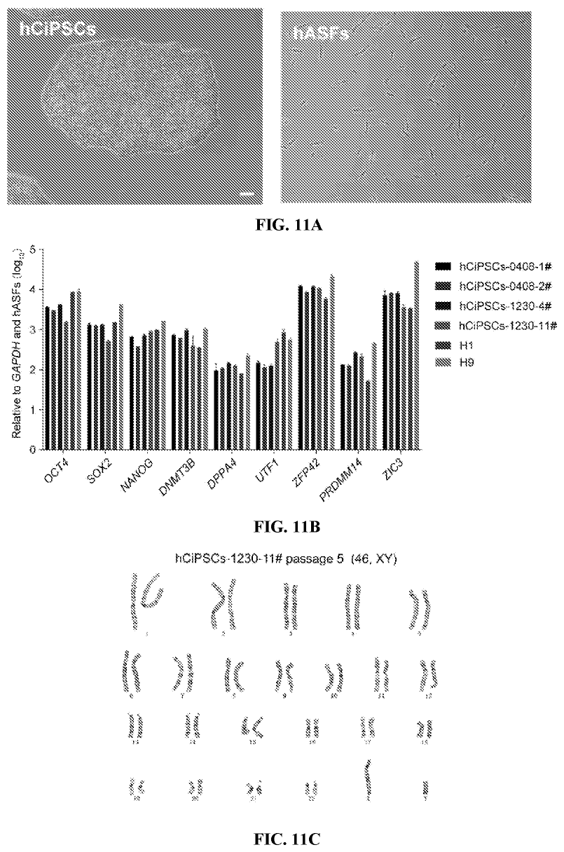

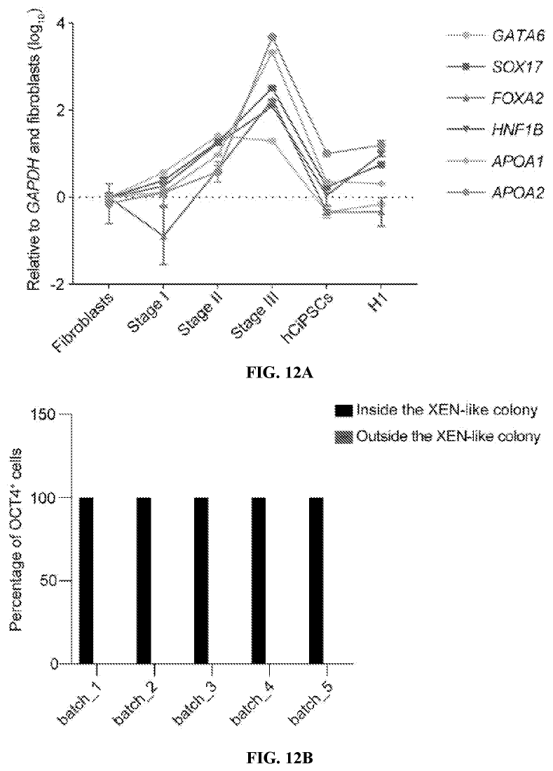

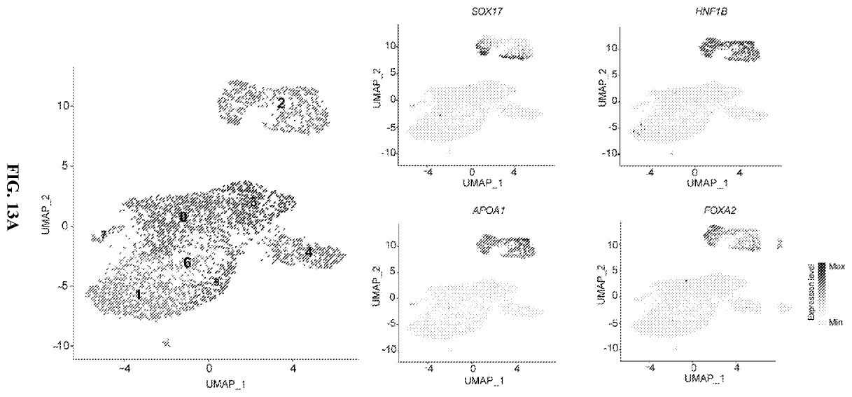

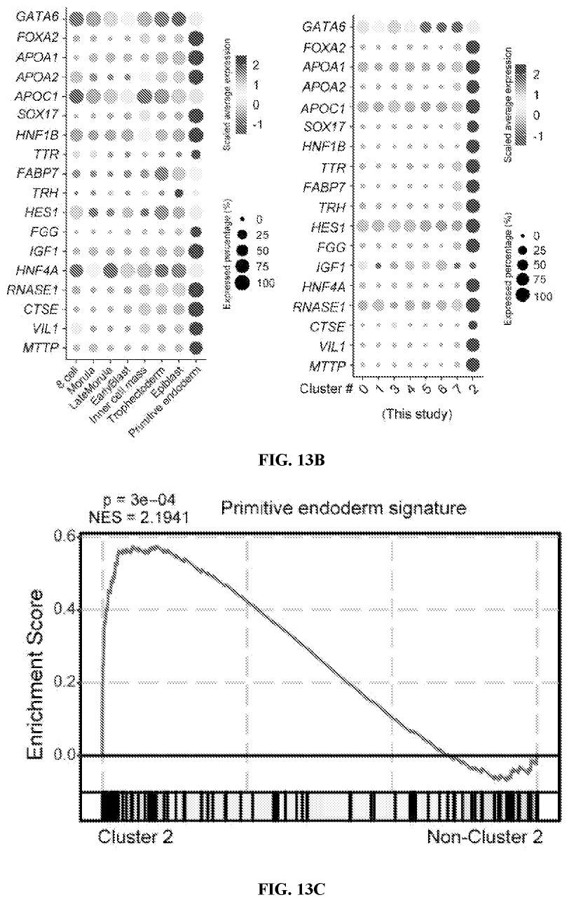

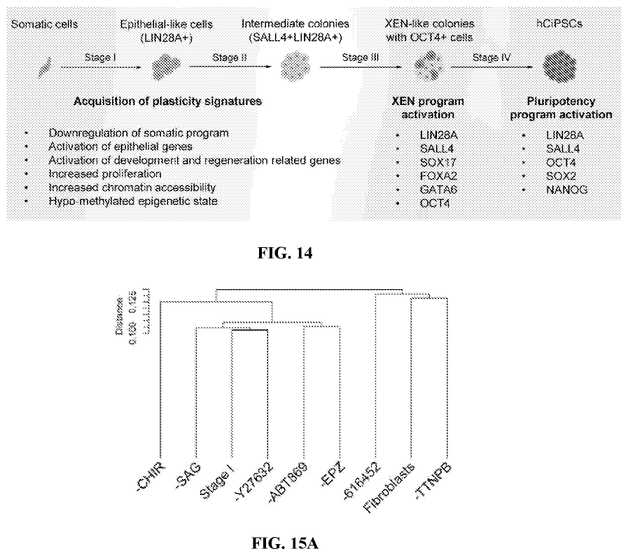

To further understand the chemical reprogramming of human cells, the gene expression was profiled at the end of each reprogramming stage. First, 3 sequential key phases were identified during the reprogramming process, beginning with the acquisition of plasticity signatures in the early stages (stage I and stage II), followed by activation of an extra-embryonic endoderm (XEN) program (stage III), and finally, establishment of an integrated pluripotency network (FIG. 14). Immunofluorescence and single-cell RNA sequencing showed a set of highly expressed XEN-related markers in stage III (FIG. 12A-B, data not shown and FIG. 13A-C). The studies also showed that the OCT4-positive cells emerged from the XEN-like colonies (FIG. 12A and data not shown), indicating that the XEN program bridged pluripotency acquisition during the late stage, as it does with chemical reprogramming of mouse cells (8-10). In the early stages, down-regulation of the somatic gene program was observed (Downregulated genes at early stage: PRRX2, COL6A2 (Collagen Type VI Alpha 2 Chain), VIM (Vimentin), COL1A1 (Collagen Type I Alpha 1 Chain), COL1A2 (Collagen Type I Alpha 2 Chain), RUNX1 (Runt-related transcription factor 1), ZEB1 (Zinc Finger E-Box Binding Homeobox 1), PRRX1 (Paired related homeobox 1), MMP3 (Matrix Metallopeptidase 3), followed by an up-regulation observed of a series of genes involved in embryonic development and regeneration (upregulated genes at early stage: FOXC1 (Forkhead Box C1), KRT19 (Keratin 19), KRT8 (Keratin 8), KRT18 (Keratin 18), CDC7 (Cell Division Cycle 7), KDR (Kinase Insert Domain Receptor), VASH2 (Vasohibin 2), MARCKS11 (MARCKS-related protein), SOX4 (MARCKS-related protein), FGF9 (Fibroblast growth factor 9), CDKNIC (Cyclin-dependent kinase inhibitor 1C), FGFR3 (Fibroblast growth factor receptor 3), LIN28A(lin-28 homolog A), BMP4 (bone morphogenetic protein 4), LIFR (Leukemia inhibitory factor receptor), CCND2 (cyclin D2), EFNB1 (ephrin B1), LEF1 (lymphoid enhancer binding factor 1), WT1(Wilms tumor 1), NOTCH1(Neurogenic locus homolog protein 1), SALL4(Spalt-Like Transcription Factor 4), IGFBP3(Insulin-like growth factor-binding protein 3), HMGA2 (High mobility group AT-hook protein 2), GATA2 (GATA-binding protein 2), MSX1(msh homeobox 1), EDN3 (endothelin 3), TBX3(T-box transcription factor 3), TBX2(T-box transcription factor 2), HOXA1(homeobox A1), ZAP70(zeta chain of T cell receptor associated protein kinase 70), HOXA5(homeobox A5), HOXB9(homeobox B), HOXA9(homeobox A), IGF2(insulin like growth factor 2), PGF(placental growth factor), PTCH1(patched 1), PROK1 (prokineticin 1), CNTFR(ciliary neurotrophic factor receptor), HEY2(hes related family bHLH transcription factor with YRPW motif 2), CDX2(caudal type homeobox 2), DLX5(distal-less homeobox 5), IGFL4(IGF like family member 4), PRAME(PRAME nuclear receptor transcriptional regulator), GLI1(GLI family zinc finger 1; see also, FIG. 3A, and FIG. 3E), all of which resembled the dedifferentiation that occurs in natural regeneration (17-19). Moreover, the increasing cell proliferation (FIG. 3A and E) represented an important cell dedifferentiation and regeneration feature (17-19). Notably, SALL4 and LIN28A, which were activated in the early stage, are key promoters of tissue repair and regeneration initiation in several organisms (20-23). Remarkably, t chromatin accessibility analyses showed that loci including the genes regulating development and pluripotency were opened at stage II (FIG. 3B and data not shown, and Fig.3G). In addition, the cells also acquired a hypo-methylated epigenetic state at stage II (FIG. 3C and D), and the promoter regions of genes related to embryonic development, cell cycle, and stem cell proliferation were de-methylated (FIG. 3E). Altogether, these results suggest the early stage induction of a less differentiated and more plastic status provided the cells permission to be reprogrammed to a pluripotent state (FIG. 14).

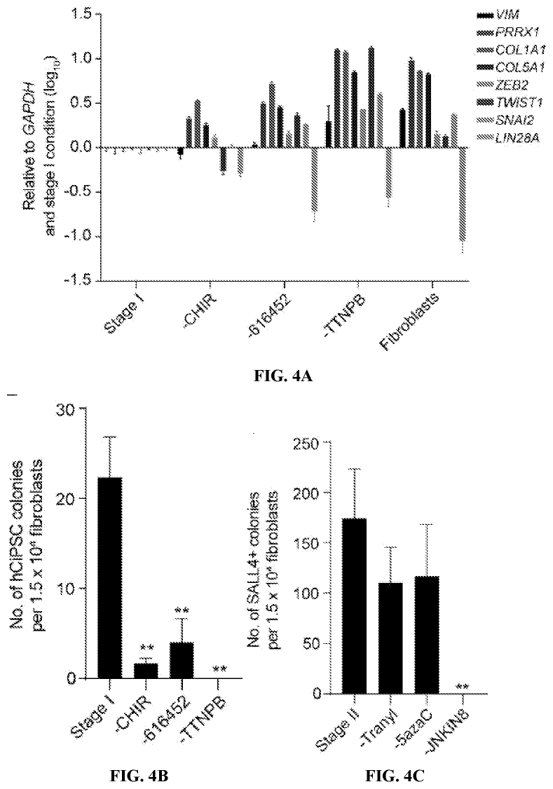

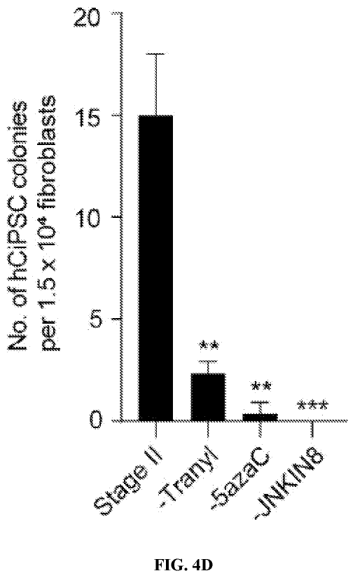

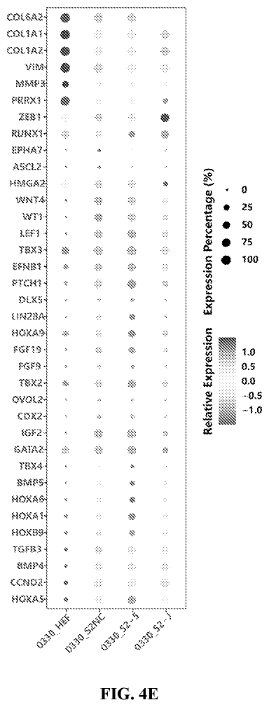

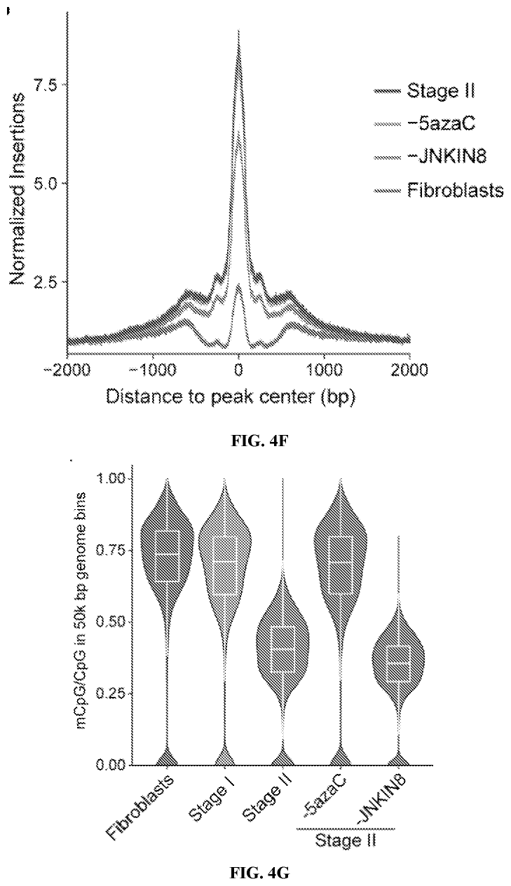

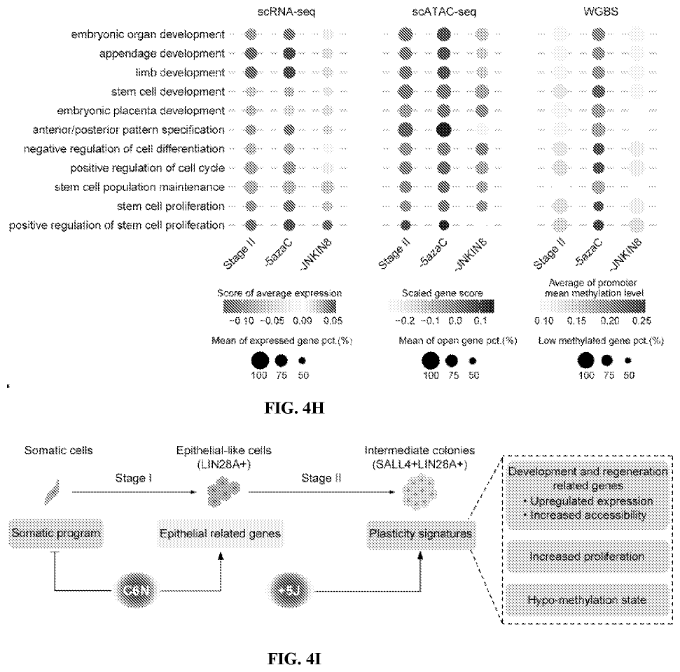

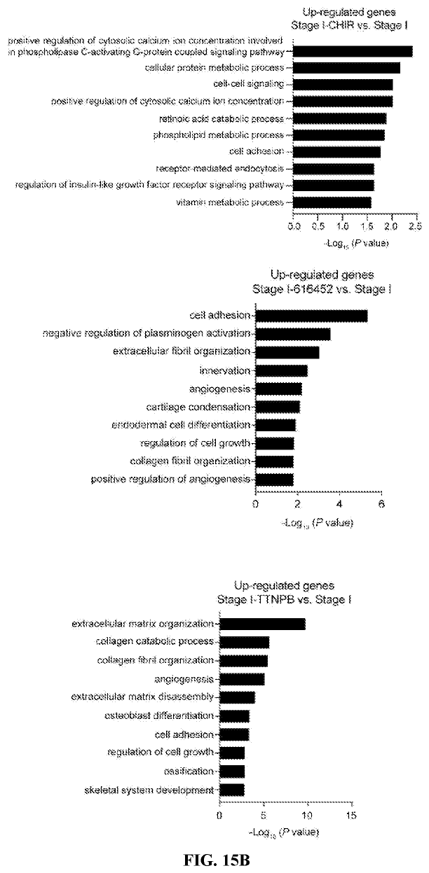

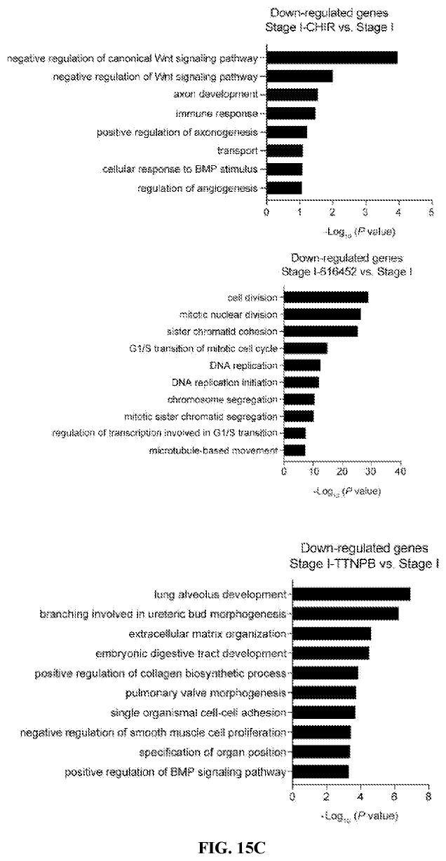

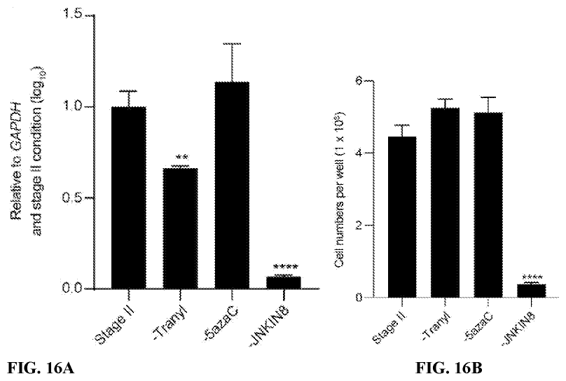

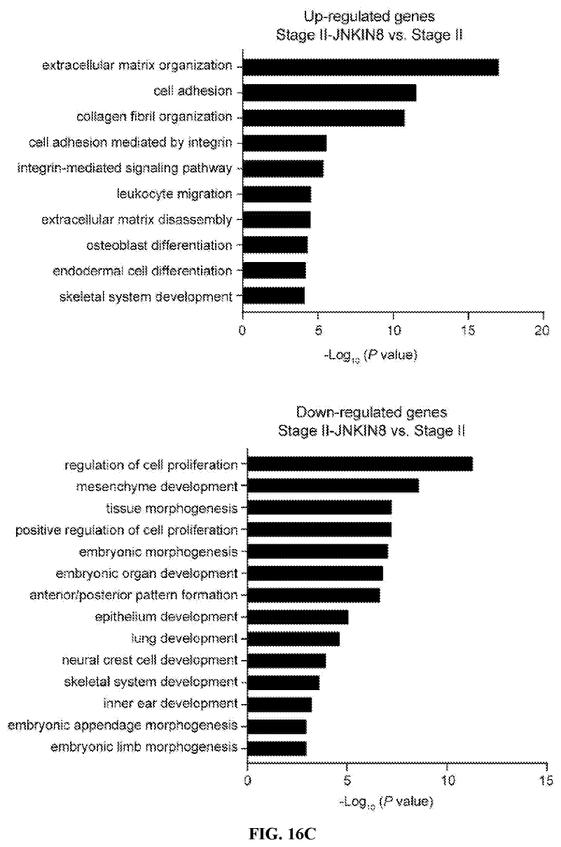

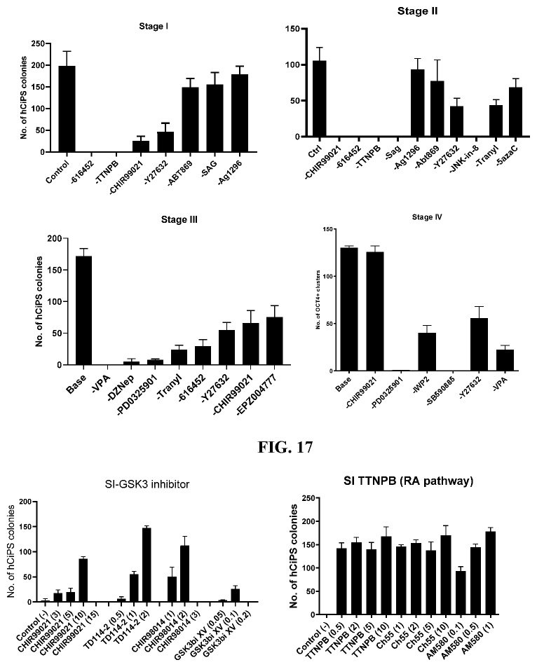

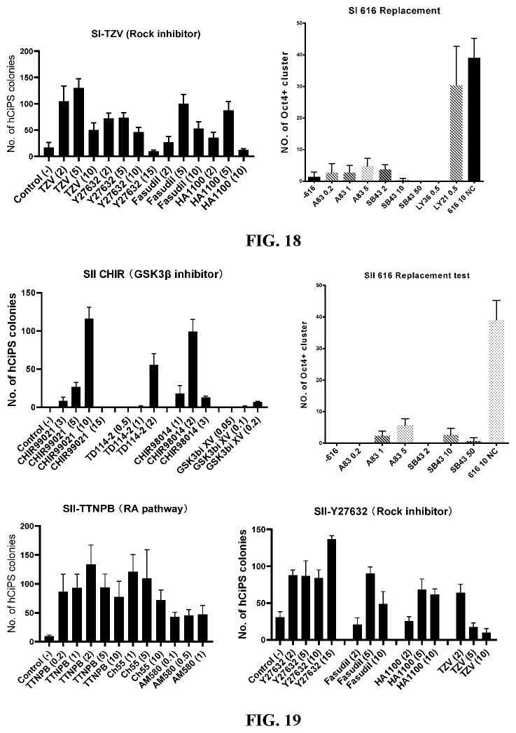

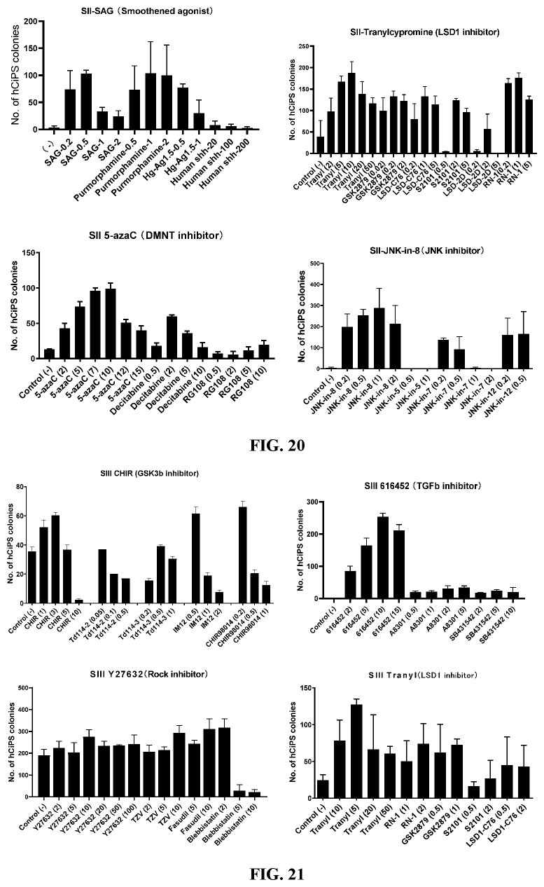

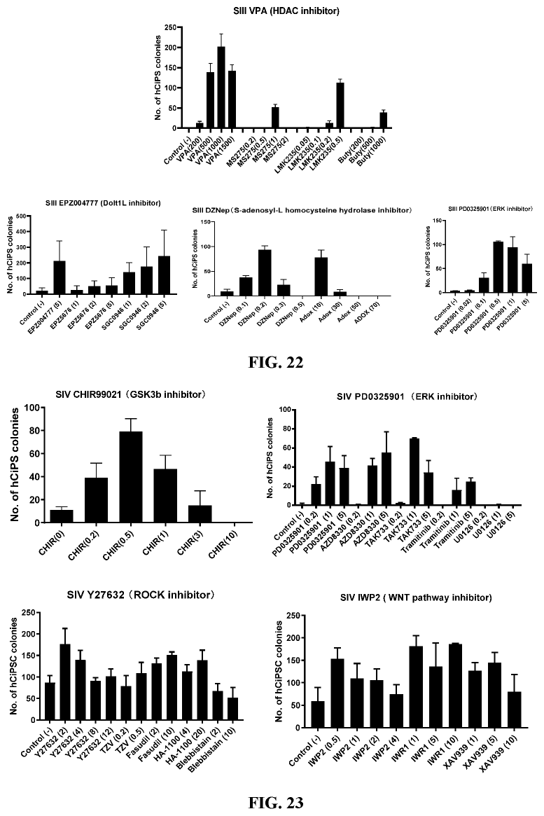

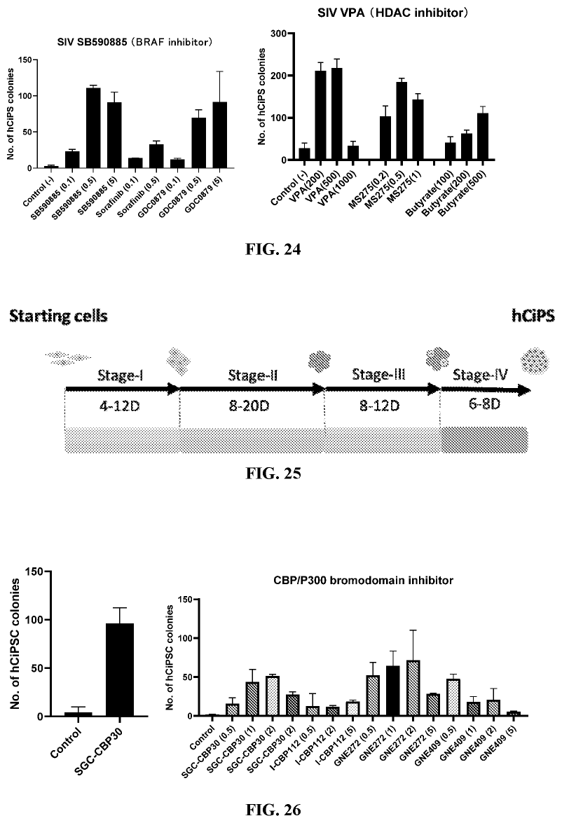

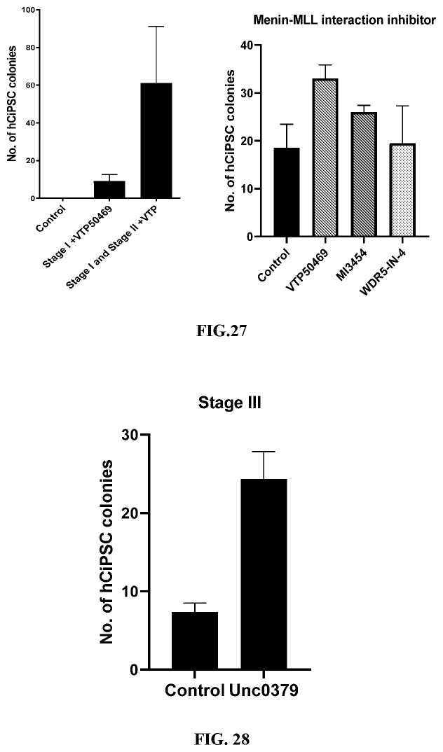

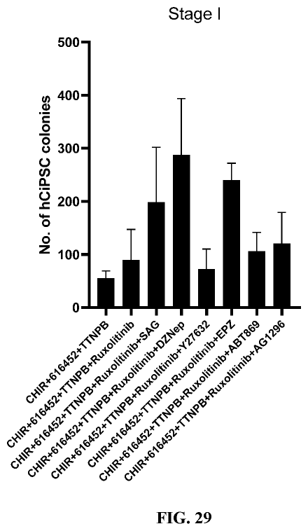

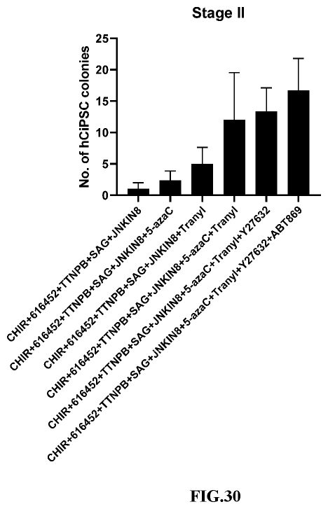

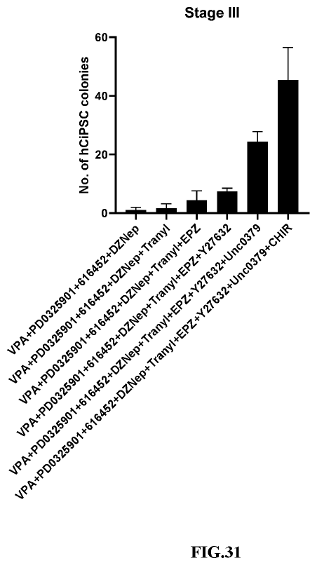

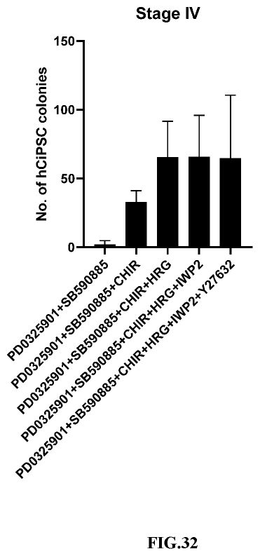

In chemical reprogramming, compared to mouse cells, liberating human somatic cell plasticity at the early stages is more challenging, which is consistent with the reports that human somatic cells that have a reduced plastic potential (13-14) are particularly refractory to external signaling stimulation (15). Using a stepwise chemical reprogramming strategy, the studies herein were able to effectively modulate human somatic cell plasticity signatures. A stepwise chemical reprogramming strategy was used to effectively modulate human somatic cell plasticity signatures. The studies showed in stage I, removing CHIR99021, 6161452 or TTNPB severely reduced LIN28A activation and inhibited the downregulation of the somatic program (FIG. 4A and FIG. 15A-C). In stage II, JNKIN8 is required for the activation of SALL4 (FIG. 4C and FIG. 16A-C) and the acquisition of plasticity signatures (data not shown and FIG. 4F, FIG. 4H and FIG. 19B and C), while the cells required 5-Azacytidine to develop a hypo-methylated state (FIG. 4G and H). In addition, chromatin accessibility analysis also confirmed that JNKIN8 is essential for the opened chromatin status of the genes that activated at stage II (FIG. 4F and H). Moreover, when any of those factors (CHIR99021, 616452, TTNPB, JNKIN8 and 5-Azacytidine) were removed, the generation of hCiPSCs was greatly impaired (FIG. 4B and D), suggesting that they were all indispensable for establishing cell plasticity for the pluripotency network. These studies demonstrated that small molecules can synergistically manipulate endogenous pathways and epigenetic targets sufficiently enough to liberate human somatic cells from a tightly locked differentiated state to a plastic state that permits cell-fate conversion (FIG. 4I). These findings provide new possibilities of therapeutic reprogramming wherein the human somatic cell plasticity potential can be rebooted in vitro and in vivo through external stimuli of a relatively simple procedure and without genetic manipulation.

In summary, these studies report improved chemical reprogramming of human somatic cells to pluripotent stem cells, which has broad applications in disease modeling, drug discovery, and regenerative medicine (24-25). In addition, the present results showed that the restricted epigenetic landscape of human somatic cells can be unlocked and converted to a pluripotent state by external chemical manipulation using a select set of small molecules which inhibit/activate key biological activities. Importantly, these results reveal a new concept for cell fate reprogramming by external chemical perturbation, which is both fundamentally different from the nuclear transfer process which required the cytoplasmic components of oocytes (26) and different from the approaches that depend on overexpressing the cell's internal transcriptional factors (27-29). Therefore, these studies provides a new platform for converting human cell fate and for exploring cellular reprogramming. Furthermore, chemical reprogramming of human somatic cells provides a new way to produce patient-specific stem cells close to clinical grade cell manufacturing (30) and the advantage of flexible small molecule combinations that allow easy adjustments for different applications in regenerative medicine.

### REFERENCES

1. Jaenisch, Nat Genet 33, 245-254 (2003).
2. Liu, et al. Nat Rev Cancer 16, 359-372 (2016).
3. Ikeuchi, et al. Development 143, 1442-1451 (2016).
4. Bosch, Dev Biol 303, 421-433 (2007).
5. Nicetto, et al. Current Opinion in Genetics & Development 55, 1-10 (2019).
6. Hawkins, et al. Cell Stem Cell 6, 479-491 (2010).
7. Hou, et al. Science 341, 651-654 (2013).
8. Zhao, et al. Cell 163, 1678-1691 (2015).
9. Ye, et al . . . Cell Research 26, 34-45 (2015).
10. Zhao, et al. Cell Stem Cell 23, 31-45.e37 (2018).
11. Xu, et al. Cell Stem Cell 16, 119-134 (2015).
12. Li, et al. Cell Stem Cell 13, 270-283 (2013).
13. Alvarado, Nature Reviews Genetics 7, 873-884 (2006).
14. Wang, C. K. Hu, A. Zeng, D. Alegre, D. Hu, K. Gotting, A. Ortega Granillo, Y. Wang, S. Robb, R. Schnittker, S. Zhang, D. Alegre, H. Li, E. Ross, N. Zhang, A. Brunet, A. Sanchez Alvarado, Changes in regeneration-responsive enhancers shape regenerative capacities in vertebrates. Science 369, (2020).
15. Akagi, Trends in Molecular Medicine 10, 542-548 (2004).
16. Kellner, et al. Histol Histopathol 25, 405-412 (2010).
17. Jopling, et al. Nature Reviews Molecular Cell Biology 12, 79-89 (2011).
18. Tanaka, Cell 165, 1598-1608 (2016).
19. Goldman, Nature Reviews Genetics 21, 511-525 (2020).
20. Neff, et al. Developmental Dynamics 240, 979-989 (2011).
21. Erickson, M. D. Gearhart, D. D. Honson, T. A. Reid, M. K. Gardner, B. S. Moriarity, K. Echeverri, A novel role for SALL4 during scar-free wound healing in axolotl. npj Regenerative Medicine 1, (2016).
22. Shyh-Chang, et al. Cell 155, 778-792 (2013).
23. Ye, Z. Su, S. Xie, Y. Liu, Y. Wang, X. Xu, Y. Zheng, M. Zhao, L. Jiang, Yap-lin28a axis targets let7-Wnt pathway to restore progenitors for initiating regeneration. eLife 9, (2020).
24. Soldner, et al. Cell 175, 615-632 (2018).
25. Rowe, et al. Nature Reviews Genetics 20, 377-388 (2019).
26. Tachibana, et al. Cell 153, 1228-1238 (2013).
27. Takahashi, et al. Cell 131, 861-872 (2007).
28. Yu, et al. Science 318, 1917-1920 (2007).
29. Park, et al. Nature 451, 141-146 (2007).
30. Li, et al. Curr Opin Genet Dev 52, 29-35 (2018).

1. A method for reprogramming human somatic cells into human chemically induced pluripotent cells (hCiPSCs) comprising:
culturing a human somatic cell in a four stage cell culture process, wherein:
(a) stage I comprises culturing the somatic cells in cell culture medium supplemented with small molecules with the following biological activities:
(i) a glycogen kinase inhibitor,
(ii) a TGFβ inhibitor, and
(iii) a retinoic acid receptor (RAR) agonist (Stage I condition), for an effective amount of time to convert the cells into a monolayer epithelial-like cells;
(b) stage II comprises culturing the cells from the stage I in cell culture medium supplemented with small molecules the following biological activities:
(i) a glycogen kinase inhibitor,
(ii) a TGFβ inhibitor,
(iii) a retinoic acid receptor (RAR) agonist,
(iv) an agonist for the G protein-coupled receptor Smoothened, and
(v) a c-Jun kinase inhibitor (Stage II condition), for an effective amount of time to upregulate one or more pluripotency-related transcriptional factors;
(c) stage III comprises culturing cells from stage II in cell culture medium supplemented with small molecules with the following biological activities:
(i) a histone acetylator/deacetylase inhibitor,
(ii) a TGFβ inhibitor,
(iii) a MAPK inhibitor, and
(iv) a SAH hydrolase inhibitor (Stage III condition), for an effective amount of time to establish an initial pluripotency network, measured by the expression of OCT4; and
(d) stage IV comprises culturing cells from stage III in cell culture medium supplemented with small molecules with the following biological activities:
(i) a B-Raf inhibitor, and
(ii) a MAPK inhibitor (Stage IV condition), for an effective amount of time to fully establish a pluripotent network, measured by co-expression OCT4, SOX2, and NANOG.

2. The method of claim 1, wherein the Stage I condition further comprises supplementing the cell culture medium with one or more small molecules with the following biological activities:
(i) a selective inhibitor of Rho-associated, coiled-coil containing protein kinase (ROCK),
(ii) a receptor tyrosine kinase inhibitor,
(iii) an agonist for the G protein-coupled receptor Smoothened,
(iv) a Dot1L inhibitor,
(v) a Jak 1/Jak2 inhibitor,
(vi) an SAH hydrolase inhibitor, and
(vii) a Menin-MLL interaction inhibitor,
and optionally, the Stage II condition further comprises supplementing the cell culture medium with one or more small molecules with the following biological activities:
(i) a DNA methyltransferase inhibitor,
(ii) inhibitor of histone demethylation,
(iii) a selective inhibitor of Rho-associated, coiled-coil containing protein kinase (ROCK),
(iv) a receptor tyrosine kinase inhibitor,
(v) a G9a inhibitor,
(vi) a BMP receptor/AMPK inhibitor,
(vii) a Jak1/Jak2 inhibitor,
(viii) a p38 MAPK inhibitor,
(ix) a CBP/p300 bromodomain inhibitor, and
(x) a Menin-MLL interaction inhibitor,
and optionally, the Stage III condition further comprises supplementing the cell culture medium with one or more small molecules with the following biological activities:
(i) a glycogen kinase inhibitor,
(ii) a selective inhibitor of Rho-associated, coiled-coil containing protein kinase (ROCK),
(iii) inhibitor of histone demethylation, and
(iv) a Dot1L inhibitor;
(v) a SETD8 inhibitor,
and optionally, the Stage IV condition further comprises supplementing the cell culture medium with one or more small molecules with the following biological activities:
(i) a Wnt inhibitor,
(ii) a glycogen kinase inhibitor,
(iii) a selective inhibitor of Rho-associated, coiled-coil containing protein kinase (ROCK), and
(iv) a histone acetylator/deacetylase inhibitor.

3. The method of claim 1, wherein the cells are maintained in the stage I condition for a period of time ranging from 4-12 days.

4. The method of claim 1, wherein the cells are maintained in the stage II condition for a period of time ranging from 8-20 days.

5. The method of claim 1, wherein the cells are maintained in the stage III condition for a period of time ranging from 8-12 days.

6. The method of claim 1, wherein the cells are maintained in the stage IV condition for a period of time ranging from 6-8 days.

7. The method of claim 1, wherein the somatic cell is an adult somatic cell and the stage I condition is performed under hypoxia characterized by with 5% O₂.

8. The method of claim 1, wherein the glycogen kinase inhibitor is selected from the group consisting of CHIR99021, SB-216763; CHIR 99021 trihydrochloride, BIO-acetoxime, GSK-3β Inhibitor XII, GSK-3 Inhibitor XV, TD114-2, TD114-3, IM12, CHIR98014 and SB-415286.

9. The method of claim 1, wherein the TGFβ inhibitor is selected from the group consisting of 616452, A 83-01, SB431542, SB 505124, GW 788388, dorsomorphine and SB 525334.

10. The method of claim 1, wherein the retinoic acid receptor (RAR) agonist is selected from the group consisting of TTNPB, Ch55 and AM580.

11. The method of claim 1, wherein the inhibitor of Rho-associated, coiled-coil containing protein kinase (ROCK) is Y27632, Fasudil or thiazovivin.

12. The method of claim 1, wherein the receptor tyrosine kinase inhibitor is selected from the group consisting of ABT 869, AG1296 and Valatanib.

13. The method of claim 1, wherein the agonist for the G protein-coupled receptor Smoothened is SAG, Purmorphamine, Hh-Ag1.5 or human SHH.

14. The method of claim 2, wherein, when present, (a) the DNA methyltransferase inhibitor is selected from the group consisting of 5-Azacytidine, Decitabine and RG108 (b) inhibitor of histone demethylation is selected from the group consisting of tranylcypromine, GSK2879, LSD-C76, S2101, RN1 and (c) the c-Jun kinase inhibitor is selected from the group consisting of JNKIN8, Sp600125; JNK-in-5; JNK-in-7; and JNK-in-12.

15. The method of claims 2, wherein, when present, (a) the Dot1L inhibitor is selected from the group consisting of EPZ004777, EPZ5676 and SGC0946, (b) the SAH hydrolase inhibitor is selected from the group consisting DZNep, Adox and NepA.

16. The method of claims 2, wherein, when present, (a) the histone acetylator/deacetylase inhibitor is selected from the group consisting of valproic acid, MS275, LMK235, Butyrate, Apicidin, CI 994, Depsipeptide, sodium,4-pehynl butyrate, sodium butyrate, and UF 010, (b) the MAPK inhibitor is selected from the group consisting of PD0325901. AZD8330; and TAK-733.

17. The method of claim 1, wherein the human somatic cell is selected from the group consisting of cells of hematological origin, skin derived cells, adipose cells, epithelial cells, endothelial cells, cells of mesenchymal origin, parenchymal cells (for example, hepatocytes), neurological cells, and connective tissue cells.

18. The method of claim 17, wherein the somatic cell is selected from the group consisting of fibroblasts and adipose-derived somatic cells (for example, adipocytes).

19. The method of claim 1, wherein, when present, (a) the B-Raf inhibitor is selected from the group consisting of SB590885, Sorafinib and GDC0879 and (b) the Wnt inhibitor is selected from the group consisting of IWP-2, WNT-C59, XAV-939 and IWR-1, optionally,
(i) the small molecules of stage I are selected from the following combination: CHIR99021+616452+TTNPB, CHIR99021+616452+CH55, CHIR99021+616452+AM580, CHIR99021+A8301+TTNPB, CHIR99021+SB431542+TTNPB, TD114-2+616452+TTNPB, CHIR98014+616452+TTNPB, GSK3bi XV+616452+TTNPB,
(ii) the small molecules of stage II are selected from the following combination: CHIR99021+616452+TTNPB+SAG+JNK-in-8, CHIR99021+616452+TTNPB+SAG+JNK-in-7, CHIR99021+616452+TTNPB+SAG+JNK-in-12, CHIR99021+616452+TTNPB+Purmorphamine+JNK-in-8, CHIR99021+616452+TTNPB+Hh-ag-1.5+JNK-in-8, CHIR99021+616452+CH55+SAG+JNK-in-8, CHIR99021+616452+AM580+SAG+JNK-in-8, CHIR99021+A8301+TTNPB+SAG+JNK-in-8, CHIR99021+SB431542+TTNPB+SAG+JNK-in-8, CHIR99021+LY2109761+TTNPB+SAG+JNK-in-8, TD114-2+616452+TTNPB+SAG+JNK-in-8, CHIR98014+616452+TTNPB+SAG+JNK-in-8, GSK3bi XV+616452+TTNPB+SAG+JNK-in-8,
(iii) the small molecules of stage III are selected from the following combination: VPA+Dznep+PD0325901+616452, VPA+Dznep+AZD8330+616452, VPA+Dznep+TAK733+616452, VPA+Dznep+Tramitinib+616452, VPA+Adox+PD0325901+616452, VPA+Nepa +PD0325901+616452, MS275+Dznep+PD0325901+616452, LMK235+Dznep+PD0325901+616452, Butyrate+Dznep+PD0325901+616452, and/or
(iv) the small molecules of stage IV are selected from the following combination: PD0325901+SB590885, PD0325901+Sorafinib, PD0325901+GDC0879, AZD8330+SB590885, AZD8330+Sorafinib, AZD8330+GDC0879, TAK733+SB590885, TAK733+Sorafinib, TAK733+GDC0879. Tramitinib+SB590885, Tramitinib+Sorafinib, Tramitinib+GDC0879.

20. Cells obtained by a method comprising one or more stages defined in claim 1, for example, the cells being
(i) epithelia-like cells obtained by culture of stage I, characterized in down-regulation of at least one gene at early stage MMP1, ZEB1, VIM, COLIA1, COL5A1, COL6A2, PRRX1, SNAI2, TWIST1 and TWIST2, and up-regulation of at least one gene relating to LIN28A and KRT, e.g., KRT8, KRT18, KRT19, and LIN28A;
(ii) plasticity state cells with regeneration program obtained by culture of stage I and stage II, characterized in that they express at least one of SALL4 and LIN28A, and have unlocked epigenome state with increased number of opened chromatin loci, and increased DNA demethylation;
(iii) XEN-like cells obtained by culture of stage I, stage II and stage III characterized in up-regulation of at least one gene LIN28A, SALL4, and OCT4, and expression of at least one of XEN (extraembryonic endoderm) related markers, e.g., GATA6, SOX17, FOXA2, HNF1B, APOA1, and APOA2; and/or
(iv) Human chemically induced pluripotent stem cells, characterized in that they express at least one surface marker TRA-1-60, TRA-1-81, and SSEA-4, along with the core pluripotency transcriptional factors OCT4, SOX2, DNMT3B, DPPA4, UTFI, ZFP42, ZIC3, and NANOG.

21. The cells of claim 20, wherein the human chemically induced pluripotent stem cells further express TRA-1-60, TRA-1-81, and/or SSEA-4.

22. The cells of claim 20, characterized in that the human chemically induced pluripotent stem cells can expand for more than 20 passages, for example, for up to 25, 30, 35, 40, 41, 42, passages in cell culture.

23. The cells of claim 20, wherein the primary human chemically induced pluripotent stem cells which induced at the end of stage SIV, express at least one factor selected factor selected from the group consisting of Developmental Pluripotency Associated 3 (DPPA3), Kruppel-Like Factor 17 (KLF17) and DNA methyltransferase 3 like (DNMT3L).

24. A cell culture media composition or kit for reprogramming human somatic cells into human chemically induced pluripotent cells, the composition or kit comprising a combination of the molecules of one or more of Stages I-IV, defined in claim 1.

25. The composition or kit of claim 24, for use in preparing cells defined in claim 20.
